SEQUENCE LISTING (213 sequences)

SEQ ID NO: 1 - protein, 650 aa, homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 2 - protein, 627 aa, homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 3 - protein, 435 aa, Homo sapiens
GHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSASLGQHPQDYTVENLIRMGVAGLVLVVLGILLFEAQHSQR

SEQ ID NO: 4 - protein, 419 aa, homo sapiens
GPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEGPRPSPTRSVSTAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYA

SEQ ID NO: 5 - protein, 574 aa, homo sapiens
GTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 6 - protein, 603 aa, homo sapiens
GPFPKPTLWAEPGSVISWGSPVTIWCQGSLEAQEYRLDKEGSPEPLDRNNPLEPKNKARFSIPSMTEHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVNPSHRWRFTCYYYYMNTPQVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYA

SEQ ID NO: 7 - protein, 411 aa, homo sapiens
GPLPKPTLWAEPGSVITQGSPVTLRCQGSLETQEYHLYREKKTALWITRIPQELVKKGQFPILSITWEHAGRYCCIYGSHTAGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSD

SEQ ID NO: 8 - protein, 471 aa, homo sapiens
ENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVE

SEQ ID NO: 9 - protein, 437 aa, homo sapiens
GPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSASHAKDYTVENLIRMGMAGLVLVFLGILLFEAQHSQRNPQD

SEQ ID NO: 10 - protein, 338 aa, homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 11 - protein, 358 aa, homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 12 - protein, 117 aa, mus musculus
QVQLQQSGAELVKPGASVKLSCKASGYTFTEHTIHWIKQRSGQGLEWIGWFYPGSGSMKYNEKFKDKATLTADKSSSTVYMELTRLTSEDSAVYFCARHTNWDFDYWGQGTTLTVSS

SEQ ID NO: 13 - protein, 111 aa, mus musculus
DIVLTQSPASLAVSLGQRATISCKASQSVDYGGDSYMNWYQQKPGQPPKLLIYAASNLESGIPARFSGSGSGTDLTLNIHPVEEDDAAMYYCQQSNEEPWTFGGGTKLEIK

SEQ ID NO: 14 - protein, 5 aa, mus musculus
EHTIH

SEQ ID NO: 15 - protein, 17 aa, mus musculus
WFYPGSGSMKYNEKFKD

SEQ ID NO: 16 - protein, 8 aa, Mus musculus
HTNWDFDY

SEQ ID NO: 17 - protein, 15 aa, Mus musculus
KASQSVDYGGDSYMN

SEQ ID NO: 18 - protein, 7 aa, Mus musculus
AASNLES

SEQ ID NO: 19 - protein, 9 aa, Mus musculus
QQSNEEPWT

SEQ ID NO: 20 - protein, 117 aa, Mus musculus
QVQLQQSGAELVKPGASVRLSCKASGYTFTAHTIHWVKQRSGQGLEWIGWLYPGSGSIKYNEKFKDKATLTADKSSSTVYMELSRLTSEDSAVYFCARHTNWDFDYWGQGTTLTVSS

SEQ ID NO: 21 - protein, 111 aa, Mus musculus
NIVLTQSPASLAVSLGQRATISCKASQSVDYGGASYMNWYQQKPGQPPKLLIYAASNLESGIPARFSGSGSGTDLTLNIHPVEEEDAAMYYCQQSNEEPWTFGGGTKLEIK

SEQ ID NO: 22 - protein, 5 aa, Mus musculus
AHTIH

SEQ ID NO: 23 - protein, 17 aa, Mus musculus
WLYPGSGSIKYNEKFKD

SEQ ID NO: 24 - protein, 8 aa, Mus musculus
HTNWDFDY

SEQ ID NO: 25 - protein, 15 aa, Mus musculus
KASQSVDYGGASYMN

SEQ ID NO: 26 - protein, 7 aa, Mus musculus
AASNLES

SEQ ID NO: 27 - protein, 9 aa, Mus musculus
QQSNEEPWT

SEQ ID NO: 28 - protein, 122 aa, Mus musculus
QVQLQQPGAELVKPGASVRMSCKASGYTFTSYWVHWVKQRPGQGLEWIGVIDPSDSYTSYNQNFKGKATLTVDTSSKTAYIHLSSLTSEDSAVYFCARGERYDGDYFAMDYWGQGTSVTVSS

SEQ ID NO: 29 - protein, 107 aa, Mus musculus
DIVMTQSPASLSVSVGETVTITCRASENIYSNLAWYQQKQGKSPQLLVYAATNLADGVPSRFSGSRSGTQYSLKINSLQSEDFGTYYCQHFWNTPRTFGGGTKLEIK

SEQ ID NO: 30 - protein, 5 aa, Mus musculus
SYWVH

SEQ ID NO: 31 - protein, 17 aa, Mus musculus
VIDPSDSYTSYNQNFKG

SEQ ID NO: 32 - protein, 13 aa, Mus musculus
GERYDGDYFAMDY

SEQ ID NO: 33 - protein, 11 aa, Mus musculus
RASENIYSNLA

SEQ ID NO: 34 - protein, 7 aa, Mus musculus
AATNLAD

SEQ ID NO: 35 - protein, 9 aa, Mus musculus
QHFWNTPRT

SEQ ID NO: 36 - protein, 118 aa, Mus musculus
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAKPRWDDYAMDYWGQGTSVTVSS

SEQ ID NO: 37 - protein, 106 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLWTFGGGTKLEIK

SEQ ID NO: 38 - protein, 120 aa, Mus musculus
EVQLQESGPGLVKPSQSLSLTCSVTGYSITSGYYWNWIRQFPENKLEWMGYIRYDGSNNYNPSLNNRISITRDASKNQFFLKLNSVTTEDTATYYCARGWLLWFYAVDYWGQGTSVTVSS

SEQ ID NO: 39 - protein, 112 aa, Mus musculus
DVVMTQTPLSLPVSLGDQASISCRSSQSIVHTNGNTYLEWYLQKSGQSPKLLIYKVSNRLSGVPDRFSGSGSGTDFTLKISRVEAEDLGIYYCFQGSHVPWTFGGGTKLEIK

SEQ ID NO: 40 - protein, 117 aa, Mus musculus
QVQLKESGPGLVAPSQSLSITCTVSGFSLTSYGVSWVRQPPGKGLEWLGVIWGDGNTNYHSALISRLSISKDNSKSQVFLKLNSLQTDDTATYYCARTNWDGWFAYWGQGTLVTVSA

SEQ ID NO: 41 - protein, 108 aa, Mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYRSYPLGTFGGGTKLEIK

SEQ ID NO: 42 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 330 aa, Artificial
  Homo sapiens with substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 138 aa, Homo sapiens
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAGALQSTASLFVVSLSLLHLYS

SEQ ID NO: 47 - protein, 141 aa, Homo sapiens
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVGALQSTASLFVVSLSLLHLYS

SEQ ID NO: 48 - protein, 139 aa, Homo sapiens
TGVHSGKPIPNPLLGLDSTSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 49 - protein, 348 aa, Homo sapiens
TGVHSGKPIPNPLLGLDSTFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 50 - protein, 239 aa, Homo sapiens
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVGALQSTASLFVVSLSLLHLYS

SEQ ID NO: 51 - protein, 240 aa, Homo sapiens
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 52 - protein, 447 aa, Homo sapiens
TGVHSGKPIPNPLLGLDSTSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 53 - protein, 338 aa, Homo sapiens
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 54 - protein, 548 aa, Homo sapiens
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 55 - protein, 98 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGA

SEQ ID NO: 56 - protein, 137 aa, homo sapiens
FYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 57 - DNA, 35 bp, homo sapiens
acaggcgtgcattcggggcacctccccaagcccac

SEQ ID NO: 58 - DNA, 58 bp, Homo sapiens
cgaggtcgggggatcctcaatggtggtgatgatggtggtgccttcccagaccactctg

SEQ ID NO: 59 - protein, 441 aa, Artificial
  synthetic
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHHHHHHH

SEQ ID NO: 60 - protein, 33 aa, Homo sapiens
ACAGGCGTGCATTCGGGGCACCTCCCCAAGCCC

SEQ ID NO: 61 - protein, 36 aa, Homo sapiens
CCGCCCCGACTCTAGACTAGTGGATGGCCAGAGTGG

SEQ ID NO: 62 - protein, 43 aa, Homo sapiens
CCAGAACACAGGATCCGCCGCCACCATGGTGGTCATGGCGCCC

SEQ ID NO: 63 - protein, 36 aa, Homo sapiens
TTTTCTAGGTCTCGAGTCAATCTGAGCTCTTCTTTC

SEQ ID NO: 64 - DNA, 92 bp, Homo sapiens
acaggcgtgcattcgggtaagcctatccctaaccctctcctcggtctcgattctacggggcacctccccaagcccaccctctgggctgagcc

SEQ ID NO: 65 - DNA, 92 bp, Homo sapiens; identical to SEQ ID NO: 64

SEQ ID NO: 66 - DNA, 93 bp, Homo sapiens
acaggcgtgcattcgggtaagcctatccctaaccctctcctcggtctcgattctacggggcccctccccaaacccaccctctgggctgagcca

SEQ ID NO: 67 - DNA, 93 bp, Homo sapiens
acaggcgtgcattcgggtaagcctatccctaaccctctcctcggtctcgattctacggggaccatccccaagcccaccctgtgggctgagcca

SEQ ID NO: 68 - DNA, 92 bp, Homo sapiens
acaggcgtgcattcgggtaagcctatccctaaccctctcctcggtctcgattctacggggcccttccccaaacccaccctctgggctgagcc

SEQ ID NO: 69 - DNA, 93 bp, Homo sapiens; identical to SEQ ID NO: 66

SEQ ID NO: 70 - DNA, 93 bp, Homo sapiens
acaggcgtgcattcgggtaagcctatccctaaccctctcctcggtctcgattctacggaaaacctacccaaacccatcctgtgggccgagcca

SEQ ID NO: 71 - DNA, 92 bp, Homo sapiens; identical to SEQ ID NO: 68

SEQ ID NO: 72 - DNA, 37 bp, Homo sapiens
ccgccccgactctagatcatctctggctgtgctgagc

SEQ ID NO: 73 - DNA, 36 bp, Homo sapiens
ccgccccgactctagactagtggatggccagagtgg

SEQ ID NO: 74 - DNA, 37 bp, Homo sapiens
ccgccccgactctagatcaggcatagacactgggctc

SEQ ID NO: 75 - DNA, 36 bp, Homo sapiens
ccgccccgactctagactagtggatggccagggtgg

SEQ ID NO: 76 - DNA, 37 bp, Homo sapiens
ccgccccgactctagatcaggcgtagatgctgggctc

SEQ ID NO: 77 - DNA, 115 bp, Homo sapiens
cgccccgactctagatcaagagtaaagatgcagaagactaagactgactacaaatagggaagcagtagattgaagagcaccctcaccagccttggagtcggacttgttttgtggt

SEQ ID NO: 78 - DNA, 37 bp, Homo sapiens
ccgccccgactctagatcactccaccactctgaaggg

SEQ ID NO: 79 - DNA, 37 bp, homo sapiens
ccgccccgactctagatcaatcttgggggtttctctg

SEQ ID NO: 80 - protein, 14 aa, artificial
  synthetic
GKPIPNPLLGLDST

SEQ ID NO: 81 - protein, 119 aa, Mus musculus
QVQLQQSGPELVKPGASVKISCKASGYSFTNFYIHWVRQRPGQGLDWIGWIFPGSGETKFNEKFKVKATLTADTSSSTAYMQLNSLTSEDSAVYFCARSWNYDARWGYWGQGTSVTVSS

SEQ ID NO: 82 - protein, 111 aa, Mus musculus
QIVLTQSPASLAVSLGQRATISCRASESIDSYGISFLHWYQQKPGQPPKLLIYRASNLESGIPARFSGSGSRPDFTLTINPVEADDVATYYCQQSNEDPFTFGSGTKLEIK

SEQ ID NO: 83 - protein, 119 aa, Mus musculus
DVQLVESGPELVKPGASVKISCKASGYSFTNYYMQWVKQRPGQGLEWIGWIFPGGGESNYNEKFKGKATLSADTSSTTAYMQLSSLTSEDSAVYFCARTWNYDARWGYWGQGTTVTVSS

SEQ ID NO: 84 - protein, 111 aa, Mus musculus
DIQMTQSPASLTVSLGQRATISCRPSENIDSYGISFMHWYQQKPGQPPKLLIYRASNLESGIPVRFSGSGSRTDFTLTINPVEADDVATYYCQQTNEDPFTFGSGTKLEIK

SEQ ID NO: 85 - protein, 119 aa, Mus musculus
EVQLKQSGPELVKPGASVKISCKASGYSFTNYYIQWVKQRPGQGLEWIGWIFPGNGETNYNEKFKGKATLTADTSSSTAYMQLSSLTSEDSAVYFCARTWNYDARWGYWGQGTSVTVSS

SEQ ID NO: 86 - protein, 111 aa, Mus musculus
DIVLTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLEIK

SEQ ID NO: 87 - protein, 119 aa, Mus musculus
QVQLQQSGPELVKPGASVKISCKASGYSFTNYYMQWVKQRPGQGLEWIGWIFPGSGESNYNEKFKGKATLSADTSSTTAYMQLSSLTSEDSAVYFCARTWNYDARWGYWGQGTSVTVSS

SEQ ID NO: 88 - protein, 111 aa, Mus musculus
DILLTQSPASLTVSLGQRATISCRANESIDSYGISFMHWYQQKPGQPPKLLIYRASNLDSGIPARFSGSGSRTDFTLTINPVEADDVATYYCQQSNEDPFTFGSGTKLEIK

SEQ ID NO: 89 - protein, 119 aa, Mus musculus
EFQLQQSGPELVKPGASVKISCKASGYSFTNYYMQWVKQRPGQGLEWIGWIFPGSGESNYNEKFKGKATLSADTSSTTAYMQLSSLTSEDSAVYFCARTWNYDARWGYWGQGTTLTVSS

SEQ ID NO: 90 - protein, 111 aa, Mus musculus
DIVMTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLEIK

SEQ ID NO: 91 - protein, 119 aa, Mus musculus
EVKLEESGPELVKPGASVKLSCKASGYTFTNYYMQWVKQRPGQGLEWIGWIFPGSGESSYNEKFKGKATLSADTSSTTAYMQLSSLTSEDSAVYFCARTWNYDARWGYWGQGTTLTVSS

SEQ ID NO: 92 - protein, 111 aa, Mus musculus
DILMTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLELK

SEQ ID NO: 93 - protein, 119 aa, Mus musculus
EVKLQQSGPELVKPGASVKISCKASGYSFTNYYIHWVKQRPGQGLEWIGWIFPGSGETNYNEKFKVKATLSADTSSTTAYMQLSSLTSEDSAVYFCARTWNYDARWGYWGQGTTLTVSS

SEQ ID NO: 94 - protein, 111 aa, Mus musculus; identical to SEQ ID NO: 92

SEQ ID NO: 95 - protein, 119 aa, Mus musculus
QVQLKESGPELVKPGASVKISCKTSGYSFTNYYIHWVKQRPGQGLEWIGWIFPGSGDTNYNEKFKGKATLTADTSSNTASMHLSSLTSEDSAVYFCARTWNYDARWGYWGQGTTLTVSS

SEQ ID NO: 96 - protein, 111 aa, Mus musculus
DVVVTQTPASLAVSLGQRATISCRASESIDSYGISFMHWYQQKPGQPPKLLIYRASNLESGIPARFSGSGSRTDFTLTINPVEADDVATYYCQQSNEDPFTFGSGTKLEIK

SEQ ID NO: 97 - protein, 119 aa, Mus musculus
EVQLQQSGPDLVKPGASVKMSCKASGYSFTNFYIHWVKQRPGQGLEWIGWIFPGNGETNYSEKFKGKATLTADTSSSTAYMQFNSLTYEDSAVYFCARTWNYDARWVYWGQGTTVTVSS

SEQ ID NO: 98 - protein, 111 aa, Mus musculus
DIVMTQSPASLAVSLGQRATISCRVSESIDSYGISFMHWYQQKSGQPPKVLIYRASTLESGIPARFSGSGSRTDFTLTINPVEADDVATYYCQQSNEDPFTFGSGTKLEIK

SEQ ID NO: 99 - protein, 119 aa, Mus musculus
EVKLQQSGPDLVKPGASVKISCKASGYSFTNFYIHWVKQRPGQGLEWIGWIFPGNGETNYSEKFKGKATLTADTSSSTAYMQFNSLTYEDSAVYFCARTWNYDARWGYWGQGTTLTVSS

SEQ ID NO: 100 - protein, 111 aa, Mus musculus
EIVLTQSPASLAVSLGQRATISCRASDGIDSYGISFMHWYQQKPGQPPTVLIYRASILESGIPARFSGSGSRTDFTLTINPVEADDVATYYCQQTNEDPFTFGSGTKLEIK

SEQ ID NO: 101 - protein, 119 aa, Mus musculus; identical to SEQ ID NO: 83

SEQ ID NO: 102 - protein, 111 aa, Mus musculus
DILLTQSPASLAVSLGQRATISCRASDGIDSYGISFMHWYQQKPGQPPTLLIYRASTLESGIPARFSGSGSRTDFTLTINPVEADDVATYYCQQTNEAPFTFGSGTKLELK

SEQ ID NO: 103 - protein, 119 aa, Mus musculus
DVQLQESGPELVKPGASVKISCKSSGYSFTNFYIHWVKQRPGQGLDWIGWIFPGTGETNFNEKFKVKAALTADTSSSTVYMQLSTLTSEDSAVYFCARSWNYDARWGYWGQGTSITVSS

SEQ ID NO: 104 - protein, 107 aa, Mus musculus
DVVMTQTPAFLAVSLGQRATISCRASESIDSYMSWYQQKPGQPPKVLIYGASNLESGIPARFSGSGSGTDFTLNIHPVEEEDAATYYCQQSNEDPWTFGGGTKLEIK

SEQ ID NO: 105 - protein, 119 aa, Mus musculus
EVQLQESGPELVKPGASVKISCKASGYSFRNYYIQWVKQRPGQGLEWIGWIFPGNYETNYNEKFKGKATLSADTSSTTAYMQLSSLTSEDSAVYFCARSWNYDARWGYWGQGTSVTVSS

SEQ ID NO: 106 - protein, 111 aa, Mus musculus
ENVLTQSPASLAVSLGQRATISCRASESIDSFGISFMHWYQQKPGQPPKLLIYRASNLESGIPARFSGSGSGPDFSLTIDPVEADDVATYYCQQSNEAPFTFGSGTKLEIK

SEQ ID NO: 107 - protein, 119 aa, Mus musculus
QVQLKQSGPELVKPGASVKISCKASGYSFTNYYIHWVKQRPGQGLEWIGWIFPGSGETNFNEKFKGKATLTADTSSSTAYMQFSSLTSEDSAVYFCARTWNYDARWGYWGQGTTVTVSS

SEQ ID NO: 108 - protein, 111 aa, Mus musculus
EIVLTQSPASLAVSLGQRATISCRASESIDSYGISFMHWYQQKPGQPPKLLIYRASNLESGIPARFSGSGSRTDFTLTINPVEADDVATYYCQQSNEDPFTFGSGTKLEIK

SEQ ID NO: 109 - protein, 119 aa, Mus musculus; identical to SEQ ID NO: 107

SEQ ID NO: 110 - protein, 111 aa, Mus musculus
DILMTQSPASLAVSLGQRATISCRASEGIDSYGISFMHWYQQKPGQPPTLLIYRASNLESGIPARFSGSGSRTDFTLTINPVEADDVATYYCQQTNEDPFTFGSGTKLEIK

SEQ ID NO: 111 - protein, 119 aa, Mus musculus
EVQLQESGPELVKPGASVKISCKTSGYSFTNYYIHWVKQRPGQGLEWIGWIFPGSGETNYNEKFKGKATLSADTSSTTAYMQLSSLTSEDSAVYFCARTWNYDARWGYWGQGTTVTVSS

SEQ ID NO: 112 - protein, 111 aa, Mus musculus
EIQMTQSPASLAVSLGQRATISCRASEGIDSYGISFMHWYQQKPGQPPTLLIYRASNLVSGIPARFSGSGSRTDFTLTINPVEADDVATYYCQQTNEDPFTFGSGTKLEIK

SEQ ID NO: 113 - protein, 119 aa, Mus musculus
DVQLQESGPDLVKPGASVKISCKASGYSFRNYYIQWVKQRPGQGLEWIGWIFPGNNETNYNEKFKGKATLSADTSSTTAYMQLSSLTSEDSAVYFCARSWNYDARWGYWGQGTTLTVSS

SEQ ID NO: 114 - protein, 111 aa, Mus musculus
EILLTQSPASLAVSLGQRATISCRASETIDSYGISFMHWYQQKPGQPPKLLIYRASNLESGIPARFSGSGSRTDFTLTINPVEADDVATYYCQQSNEDPFTFGSGTKLEIK

SEQ ID NO: 115 - protein, 119 aa, Mus musculus
QVQLKESGPELVKPGASVKISCKASGYSFTNYYIHWVKQRPGQGLEWIGWIFPGSGETNYSEKFKGEAILTADTSSNTAYMQLSSLTSEDSAVYFCARSWNYDARWGYWGQGTTLTVSS

SEQ ID NO: 116 - protein, 111 aa, Mus musculus
EIVLTQSPASLAVSLGQRATISCRASEIIDSYGISFMHWYQQKPGQPPKLLIYRASNLESGIPARFSGSGSRTDFTLTINPVEADDVATYYCQQSNEDPFTFGSGTKLEIK

SEQ ID NO: 117 - protein, 119 aa, Mus musculus
QIQLQQSGPELVKPGASVKISCKASGYSFTNYYIQWVKQRPGQGLEWIGWIFPGSGETNYNENFKAKATLSADTSSTTAYMQLSSLTSEDSAVYFCARTWNYDARWGYWGQGTSVTVSS

SEQ ID NO: 118 - protein, 111 aa, Mus musculus
QIVLSQSPVSLAVSPGQRATISCRASESIDSYGISFMHWYKQKPGQPPKLLIYRASNLESGIPARFSGSGSRTDFTLTINPVEADDVATYYCQQSNEDPFTFGSGTKLEIK

SEQ ID NO: 119 - protein, 119 aa, Mus musculus
EVHLQQSGPELVKPGASVKISCKASGYSFTNYYIQWVKQRPGQGLEWIGWIFPGSGETNYNENFRAKATLSADTSSTTAYMQLSSLTSEDSAVYFCARTWNYDARWGYWGQGTTVTVSS

SEQ ID NO: 120 - protein, 112 aa, Mus musculus
EILLTQSPPASLAVSLGQRVTISCRPSENIDSYGISFMHWYQQKPGQPPKLLIYRASNLESGIPVRFSGSGSRTDFTLTINPVEADDVATYYCQQSNEDPFTFGSGTKLEIK

SEQ ID NO: 121 - protein, 119 aa, Mus musculus
QVQLKESGPELVKPGASVKISCKTSGYIFTNYYIHWVKQRPGQGLEWIGWIFPGSGDTNYNEKFKGKATLTADTSSSTASMQLSSLTSEDSAVYFCARTWNYDARWGYWGQGTTVTVSS

SEQ ID NO: 122 - protein, 111 aa, Mus musculus
DILLTQSPASLAVSLGQRATISCRPSENIDSYGISFMHWCQQKPGQPPKLLIYRASNLESGIPVRFSGSGSRTDFTLTINPVEADDVATYYCQQSNEDPFTFGSGTKLEIK

SEQ ID NO: 123 - protein, 119 aa, Mus musculus
QVQLKESGPELVKPGASVKISCKASGYSFTNYYIHWVKQRPGQGLEWIGWIFLGSGETNYNEKFKGEAILTADTSSTTAYMQLSSLTSEDSAVYFCARSWNYDARWGYWGQGTTLTVSS

SEQ ID NO: 124 - protein, 111 aa, Mus musculus
DILLTQSPASLAVSLGQRATISCRVSESIDSYGISFMHWYQQKSGQPPKVLIYRASTLESGIPARFSGSGSRTDFTLTINPVEADDVATYYCQQSNEDPFTFGSGTKLEIK

SEQ ID NO: 125 - protein, 119 aa, Mus musculus
EVQLQQSGPELVKPGASVKISCKASGYSFTNFYIHWVKQRPGQGLDWIGWIFPGSGETNYNERFKGKATLTSDTSSSTAYMQLSSLTSEDSAVYFCARSWNYDARWGYWGQGTTLTVSS

SEQ ID NO: 126 - protein, 111 aa, Mus musculus
EIVLTQSPASLAVSLGQRATISCRASESIDSYGISFIHWYQQKPGQPPKLLIYRASNLESGIPARFSGSGSRTDFTLTINPVEAEDVATYYCQQSNEDPFTFGSGTKLEIK

SEQ ID NO: 127 - protein, 119 aa, Mus musculus
EVQLQQSGPELVKPGASVKISCKSSGYSFTNFYIHWVKQRPGQGLDWIGWIFPGSGETNFNEKFKVKAALTADTSSNTAYMQLSSLTSEDSAVYFCARSWNYDARWGYWGQGTTVTVSS

SEQ ID NO: 128 - protein, 111 aa, Mus musculus
QIVLTQTPASLAVSLGQRATISCRASESIDSYGISFMHWYQQKPGQPPKLLIYRASNLESGIPARFSGSGSRPDFTLTINPVEADDVATYYCQQSNEDPFTFGSGTKLEIK

SEQ ID NO: 129 - protein, 118 aa, Mus musculus
QVQLKESGPGLVAPSQSLSITCTVSGFSLTSYGVSWVRQPPGKGLEWLGIIWGDGSTNYHSALVSRLSISKDNSKSQVFLKLNSLQTDDTATYYCAKPNWDYYAMDYWGQGTSVTVSS

SEQ ID NO: 130 - protein, 111 aa, Mus musculus
DAVMTQTPASLAVSLGQRATISCRASESVDSYGISFMHWYQQKPGQPPKLLIYRASILESGIPARFSGSGSRPDFSLTINPVEADDVATYYCQQSNEDPFTFGSGTKLEIK

SEQ ID NO: 131 - protein, 119 aa, Mus musculus; identical to SEQ ID NO: 103

SEQ ID NO: 132 - protein, 107 aa, Mus musculus
DIQMTQTTSSLSASLGDRVTISCRASQDISNYLNWYQQKVDGTVKLLISYTSRLHSGVPSRFSGSGSGTDYSLTISNLEQEDIATYFCQQGITLPLTFGAGTKLELK

SEQ ID NO: 133 - protein, 119 aa, Mus musculus; identical to SEQ ID NO: 99

SEQ ID NO: 134 - protein, 111 aa, Mus musculus
DVVMTQTPASLAVSLGQRATISCRASDGIDSYGISFMRWYQQKPGQPPTLLIYRASTLESGIPARFSGSGSRTNFTLTINPVEADDVATYYCQQTNEDPFTFGSGTKLEIK

SEQ ID NO: 135 - protein, 119 aa, Mus musculus
QRELQQSGPELVKPGASVNISCKASGYSFTNHYINWVKQRPGQGLEWIGWIFPGNGDTNYNEKFKGKATLTADTSSSTAYMQLSSLTSEDSAVYFCARTWNYDARWGYWGQGTTVTVSS

SEQ ID NO: 136 - protein, 111 aa, Mus musculus
DVVMTQTPAFLAVSLGQRATISCRASESIDSYGISFMHWYQQKPGQPPKVLIYRTSNLESGIPARFSGSGSRTDFTLTINPVEADDVATYYCQQSNEDPFTFGSGTKLEIK

SEQ ID NO: 137 - protein, 119 aa, Mus musculus
QVQLKESGPELVKPGTSVKISCKASGYNFRNYYIQWVKQRPGQGLEWIGWIFPGNNETNYNEKFKGKATLSADTSSTTAYMQLSSLTSEDSAVYFCARSWNYDARWGYWGQGTTVTVSS

SEQ ID NO: 138 - protein, 111 aa, Mus musculus
DVVMTQTPASLAVSLGQRATISCRASEIIDNYGISFIHWYQQKPGQPPKLLIYRASNLESGIPARFSGSGSRTDSTLTINPVGADDVATYYCQQSNEDPFTFGSGTKLELK

SEQ ID NO: 139 - protein, 5 aa, Mus musculus
NYYMQ

SEQ ID NO: 140 - protein, 17 aa, Mus musculus
WIFPGSGESSYNEKFKG

SEQ ID NO: 141 - protein, 10 aa, Mus musculus
TWNYDARWGY

SEQ ID NO: 142 - protein, 15 aa, Mus musculus
IPSESIDSYGISFMH

SEQ ID NO: 143 - protein, 7 aa, Mus musculus
RASNLES

SEQ ID NO: 144 - protein, 9 aa, Mus musculus
QQSNEDPFT

SEQ ID NO: 145 - protein, 5 aa, Mus musculus
NFYIH

SEQ ID NO: 146 - protein, 17 aa, Mus musculus
WIFPGSGETKFNEKFKV

SEQ ID NO: 147 - protein, 10 aa, Mus musculus
SWNYDARWGY

SEQ ID NO: 148 - protein, 15 aa, Mus musculus
RASESIDSYGISFLH

SEQ ID NO: 149 - protein, 7 aa, Mus musculus
RASNLES

SEQ ID NO: 150 - protein, 9 aa, Mus musculus
QQSNEDPFT

SEQ ID NO: 151 - protein, 5 aa, Mus musculus
NYYVQ

SEQ ID NO: 152 - protein, 17 aa, Mus musculus
WIFPGSGETNYNEKFKA

SEQ ID NO: 153 - protein, 9 aa, Mus musculus
QQTNEDPFT

SEQ ID NO: 154 - protein, 5 aa, Mus musculus
NYYMQ

SEQ ID NO: 155 - protein, 17 aa, Mus musculus
WIFPGGGESNYNEKFKG

SEQ ID NO: 156 - protein, 15 aa, Mus musculus
IPSESIDSYGISFMH

SEQ ID NO: 157 - protein, 5 aa, Mus musculus
NYYIQ

SEQ ID NO: 158 - protein, 17 aa, Mus musculus
WIFPGNGETNYNEKFKG

SEQ ID NO: 159 - protein, 15 aa, Mus musculus
RANESIDSYGISFMH

SEQ ID NO: 160 - protein, 7 aa, Mus musculus
RASNLDS

SEQ ID NO: 161 - protein, 17 aa, Mus musculus
WIFPGSGESNYNEKFKG

SEQ ID NO: 162 - protein, 15 aa, Mus musculus
IPSESIDSYGISFMH

SEQ ID NO: 163 - protein, 5 aa, Mus musculus
NYYIH

SEQ ID NO: 164 - protein, 17 aa, Mus musculus
WIFPGSGETNYNEKFKV

SEQ ID NO: 165 - protein, 15 aa, Mus musculus
RASESIDSYGISFMH

SEQ ID NO: 166 - protein, 17 aa, Mus musculus
WIFPGSGDTNYNEKFKG

SEQ ID NO: 167 - protein, 15 aa, Mus musculus
RVSESIDSYGISFMH

SEQ ID NO: 168 - protein, 7 aa, Mus musculus
RASTLES

SEQ ID NO: 169 - protein, 17 aa, Mus musculus
WIFPGNGETNYSEKFKG

SEQ ID NO: 170 - protein, 10 aa, Mus musculus
TWNYDARWVY

SEQ ID NO: 171 - protein, 15 aa, Mus musculus
RASDGIDSYGISFMH

SEQ ID NO: 172 - protein, 7 aa, Mus musculus
RASILES

SEQ ID NO: 173 - protein, 17 aa, Mus musculus
WIFPGNGETNYSEKFKG

SEQ ID NO: 174 - protein, 15 aa, Mus musculus
RASDGIDSYGISFMH

SEQ ID NO: 175 - protein, 9 aa, Mus musculus
QQTNEAPFT

SEQ ID NO: 176 - protein, 5 aa, Mus musculus
NYYIN

SEQ ID NO: 177 - protein, 17 aa, Mus musculus
WIFPGNGDTNYNEKFKG

SEQ ID NO: 178 - protein, 11 aa, Mus musculus
RASESIDSYMS

SEQ ID NO: 179 - protein, 7 aa, Mus musculus
GASNLES

SEQ ID NO: 180 - protein, 9 aa, Mus musculus
QQSNEDPWT

SEQ ID NO: 181 - protein, 15 aa, Mus musculus
RPSENIDSYGISFMH

SEQ ID NO: 182 - protein, 17 aa, Mus musculus
WIFPGTGETNFNEKFKV

SEQ ID NO: 183 - protein, 10 aa, Mus musculus
SWNYDARWGY

SEQ ID NO: 184 - protein, 15 aa, Mus musculus
RASESIDSFGISFMH

SEQ ID NO: 185 - protein, 9 aa, Mus musculus
QQSNEAPFT

SEQ ID NO: 186 - protein, 17 aa, Mus musculus
WIFPGSGETNFNEKFKG

SEQ ID NO: 187 - protein, 15 aa, Mus musculus
RASESIDSYGISFMH

SEQ ID NO: 188 - protein, 17 aa, Mus musculus
WIFPGSGETNFNEKFKG

SEQ ID NO: 189 - protein, 15 aa, Mus musculus
RASESIDSYGISFMH

SEQ ID NO: 190 - protein, 7 aa, Mus musculus
RASNLVS

SEQ ID NO: 191 - protein, 17 aa, Mus musculus
WIFPGSGETNYNEKFKG

SEQ ID NO: 192 - protein, 15 aa, Mus musculus
RASETIDSYGISFMH

SEQ ID NO: 193 - protein, 17 aa, Mus musculus
WIFPGNNETNYNEKFKG

SEQ ID NO: 194 - protein, 15 aa, Mus musculus
RASEIIDSYGISFMH

SEQ ID NO: 195 - protein, 17 aa, Mus musculus
WIFPGSGETNYSEKFKG

SEQ ID NO: 196 - protein, 15 aa, Mus musculus
RASESIDSYGISFMH

SEQ ID NO: 197 - protein, 17 aa, Mus musculus
WIFPGSGETNYNENFKA

SEQ ID NO: 198 - protein, 17 aa, Mus musculus
WIFPGSGETNYNENFRA

SEQ ID NO: 199 - protein, 15 aa, Mus musculus
RPSENIDSYGISFMH

SEQ ID NO: 200 - protein, 17 aa, Mus musculus
WIFPGSGDTNYNEKFKG

SEQ ID NO: 201 - protein, 15 aa, Mus musculus
RVSESIDSYGISFMH

SEQ ID NO: 202 - protein, 17 aa, Mus musculus
WIFLGSGETNYNEKFKG

SEQ ID NO: 203 - protein, 15 aa, Mus musculus
RASESIDSYGISFIH

SEQ ID NO: 204 - protein, 17 aa, Mus musculus
WIFPGSGETNYNERFKG

SEQ ID NO: 205 - protein, 15 aa, Mus musculus
RASESIDSYGISFMH

SEQ ID NO: 206 - protein, 17 aa, Mus musculus
WIFPGSGETNFNEKFKV

SEQ ID NO: 207 - protein, 15 aa, Mus musculus
RASESVDSYGISFMH

SEQ ID NO: 208 - protein, 5 aa, Mus musculus
SYGVS

SEQ ID NO: 209 - protein, 16 aa, Mus musculus
IIWGDGSTNYHSALVS

SEQ ID NO: 210 - protein, 10 aa, Mus musculus
PNWDYYAMDY

SEQ ID NO: 211 - protein, 11 aa, Mus musculus
RASQDISNYLN

SEQ ID NO: 212 - protein, 7 aa, Mus musculus
YTSRLHS

SEQ ID NO: 213 - protein, 9 aa, Mus musculus
QQGITLPLT

(19) United States
(12) Patent Application Publication
(10) Pub. No.: US 2022/0025056 A1
(43) Pub. Date: Jan. 27, 2022

(54) LEUCOCYTE IMMUNOGLOBULIN-LIKE RECEPTOR NEUTRALIZING ANTIBODIES

(71) Applicant: INNATE PHARMA, MARSEILLE (FR)

(72) Inventors: OLIVIER BENAC, MARSEILLE (FR); STÉPHANIE CHANTEUX, MARSEILLE (FR); IVAN PERROT, CASSIS (FR); BENJAMIN ROSSI, MARSEILLE (FR); NICOLAS VIAUD, AUBAGNE (FR)

(21) Appl. No.: 17/418,269

(22) PCT Filed: Dec. 20, 2019
(86) PCT No.: PCT/EP2019/086858

§ 371 Date: Jun. 25, 2021

Related U.S. Application Data
(60) Provisional application No. 62/784,862, filed on Dec. 26, 2018.

(51) Int. Cl. C07K 16/28; C07K 16/30; A61P 35/00

(52) U.S. Cl. CPC C07K 16/2863; C07K 16/2887; C07K 16/30; A61P 35/00; C07K 2317/34; A61K 2039/505; C07K 2317/71; C07K 2317/732; C07K 2317/76; C07K 2317/92; C07K 2317/565; C07K 2317/52

(57) ABSTRACT

This invention relates to agents that bind and neutralize the inhibitory activity of human ILT2 proteins having inhibitory activity in NK cells, T cells and/or other immune cells. Such agents can be used for the treatment of cancers or infectious disease.

### CROSS-REFERENCE TO RELATED APPLICATIONS

This application claims the benefit of U.S. Provisional Application No. 62/784,862 filed 26 Dec. 2018; which is incorporated herein by reference in its entirety; including any drawings.

### REFERENCE TO SEQUENCE LISTING

The present application is being filed along with a Sequence Listing in electronic format. The Sequence Listing is provided as a file entitled “LILRB1_ST25”, created 20 Dec. 2019, which is 178 KB in size. The information in the electronic format of the Sequence Listing is incorporated herein by reference in its entirety.

### FIELD OF THE INVENTION

This invention relates to agents that bind human ILT2 proteins having inhibitory activity in NK cells, T cells, monocytes, macrophages and/or other immune cells, and that neutralize the inhibitory activity of such ILT2 proteins. Such agents can be used for the treatment of cancers or infectious disease.

### BACKGROUND OF THE INVENTION

Ig-like transcripts (ILTs), also called lymphocyte inhibitory receptors or leukocyte immunoglobulin-(Ig—) like receptors (LIR/LILRs) that correspond to CD85. This family of proteins is encoded by more than 10 genes located in the 19q13.4 chromosome, and includes both activating and inhibitory members. Inhibitory LILRs transmit signals through their long cytoplasmic tails, which contain between two and four immunoreceptor tyrosine-based inhibitory domains (ITIMs) that, upon phosphorylation, recruit SHP-1 and SHP-2 phosphatases which mediate inhibition of various intracellular signal pathways. ILT-2 is a receptor for class I MHC antigens and recognizes a broad spectrum of HLA-A, HLA-B, HLA-C and HLA-G alleles. ILT-2 (LILRB1) is also a receptor for H301/UL18, a human cytomegalovirus class I MHC homolog. Ligand binding results in inhibitory signals and down-regulation of the immune response.

In addition to expression on dendritic cells (DCs), ILT2 proteins have also been reported to be expressed in NK cells. NK cells are mononuclear cell that develop in the bone marrow from lymphoid progenitors, and morphological features and biological properties typically include the expression of the cluster determinants (CDs) CD16, CD56, and/or CD57; the absence of the alpha/beta or gamma/delta TCR complex on the cell surface; the ability to bind to and kill target cells that fail to express “self” major histocompatibility complex (MHC)/human leukocyte antigen (HLA) proteins; and the ability to kill tumor cells or other diseased cells that express ligands for activating NK receptors. NK cells are characterized by their ability to bind and kill several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Normal, healthy cells are protected from lysis by NK cells.

Based on their biological properties, various therapeutic strategies have been proposed in the art that rely on a modulation of NK cells. However, NK cell activity is regulated by a complex mechanism that involves both stimulating and inhibitory signals. Briefly, the lytic activity of NK cells is regulated by various cell surface receptors that transduce either positive or negative intracellular signals upon interaction with ligands on the target cell. The balance between positive and negative signals transmitted via these receptors determines whether or not a target cell is lysed (killed) by a NK cell. NK cell stimulatory signals can be mediated by Natural Cytotoxicity Receptors (NCR) such as NKp30, NKp44, and NKp46; as well as NKG2C receptors, NKG2D receptors, certain activating Killer Ig-like Receptors (KIRs), and other activating NK receptors (Lanier, Annual Review of Immunology 2005; 23: 225-74).

Based on their biological properties, various strategies have been proposed in the art that rely on a modulation of ILT family members, notably vaccination strategies including inhibitors of ILT to relieve ILT-mediated tolerance in dendritic cells. The ILT family and its ligands are also of interest in view of reports correlating HLA-G with inhibition of immune cells such as NK cells. Wan et al. (Cell Physiol Biochem 2017; 44:1828-1841) reported that HLA-G, a natural ligand of several immune receptors including ILT2, ILT4 and KIR2DL4, can inhibit the function of many immune cells by binding to cell surface-expressed receptors.

The interactions of HLA class I molecules with ILT proteins is complex. HLA-G binds not only to ILT2 but also to ILT4 and other receptor (e.g. of the KIR family). Furthermore, many isoforms of HLA-G exist, and only the form HLA-G1 that associates with beta-2-microglobulin (and its soluble/secreted form HLA-G7) associate with bind to ILT2, whereas all forms HLA-G1, -G2, -G3, -G4, -G5, -G6 and -G7 associate with ILT4. Likewise, ILT2 and ILT4 bind not only HLA-G, but also to other MHC class I molecules. ILT2 and ILT4 use their two membrane distal domains (D1 and D2) to recognize the α3 domain and 62 m subunit of MHC molecules, both of which are conserved among classical and non-classical MHC class I molecules. Kirwan and Burshtyn (J Immunol 2005; 175: 5006-5015) reported that while ILT2 was found to have an inhibitory role on NK cell lines made to overexpress ILT2, the amount of ILT2 on normal (primary) NK cells is held below the threshold that would allow direct recognition of most MHC—I alleles. The authors consequently propose that in normal NK cells ILT2 is not active on its own but could cooperate with inhibitory KIR receptors to increase the functional range of KIRs' interaction with HLA-C molecules. More recently, Heidenreich et al. 2012 (Clinical and Developmental Immunology. Volume 2012, Article ID 652130)) concluded that ILT2 alone does not directly influence NK-cell-mediated cytotoxicity against myeloma.

Various groups have proposed to treat cancer by using antibodies or other agents that bind or target HLA-G, thereby removing the HLA-G-mediated immunosuppression and blocking of all the ILT and other receptors that interact with HLA-G such as ILT2, ILT4, KIR2DL4 and/or others (see, e.g., WO2018/091580). However, targeting HLA-G does not inhibit the interaction (if any) of ILT2 with other HLA class I ligands of ILT proteins. Despite the interest in ILT receptors related to the proposed role of HLA-G in tumor escape, there has been no clinical development of therapeutic agents that provide inhibition of ILT2.

Despite the recent advances achieved through the use of immunotherapeutic agents, there is a great unmet need for significant improvement in the treatment of cancer. Renal Cell Carcinoma (RCC) is one particular example. RCC is the most common type of kidney cancer in adults, in which it is responsible for approximately 90-95% of cases. Renal Cell Carcinoma typically originates in the lining of the proximal convoluted tubule. Unlike many other cancers, Renal Cell Carcinoma is not a single entity, but is instead composed of different cell and tumor types derived from distinct parts of the nephron (such as the epithelium and/or renal tubules), each of which have distinct genotypes, gene expression profiles, histological features and clinical phenotypes. Mortality is approximately 40%, and five-year survival for those with metastatic Renal Cell Carcinoma is less than 10%. There is a great unmet need for significant improvement in the treatment of localized and metastatic cancer as the disease remains the most lethal of all urological malignancies.

### SUMMARY OF THE INVENTION

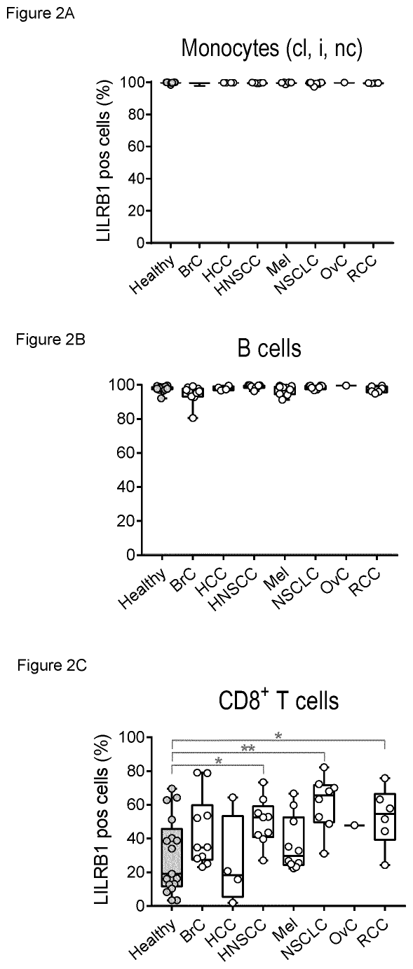

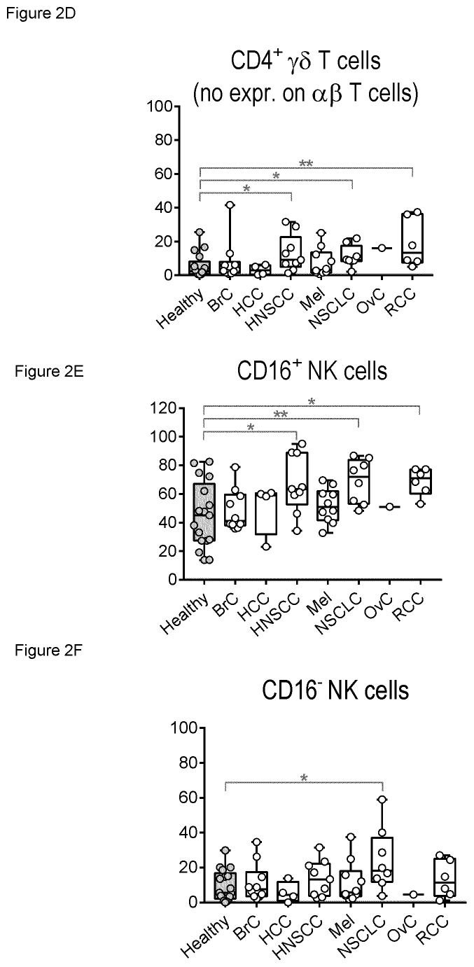

ILT2 is expressed on all monocytes and B cells, but not or only very low levels in CD4 T cells and CD16-negative NK cells. In the cytotoxic lymphocytes NK cells and CD8 T cells that express CD16 (CD16+ cells), ILT2 is expressed, but at levels that are much lower than in monocytes and B cells, both in healthy donor and cancer patients (see Examples 1 and 2). Interestingly, however, as shown in Example 2 and FIG. 2, ILT2 expression on circulating NK and CD8 T cells is particularly increased in head and neck squamous cell carcinoma (HNSCC), lung cancer (e.g. NSCLC), renal cell cancer (RCC), and ovarian cancer. Such cancers may be particularly subject to immunosuppression in which ILT2 plays a role. Consequently, the disclosure provides in one aspect that antibodies that neutralize the inhibitory activity of ILT2 can be used advantageously to treat such cancers. As shown herein, antibodies that neutralize the inhibitory activity of ILT2 show efficacy in cells from human donors having urothelial carcinoma, also known as transitional cell carcinoma (TCC).

In another aspect, the present disclosure provides antibodies and antigen binding domains that block human ILT2 and potentiate NK cell cytotoxicity in primary NK cells towards tumor cells (NK cells have relatively low levels of ILT2 expression compared to monocytes, B cells, or generally cells engineered to express ILT2). The antibodies and antigen binding domains may be particularly advantageous in treatment in a broad range of cancers, including in cancers and/or individuals having cancer who do not have increased expression of ILT2 on NK and/or CD8T cells (e.g. in circulation or tumor-infiltrating NK or T cells). The antibodies and antigen binding domains can furthermore be particularly useful in the treatment of a wide range of cancers characterized by tumor cells that express HLA-G (and/or other ILT2 ligands such as HLA-A2). The antibodies tested were able to cause primary NK cells to lyse HLA-G-expressing tumor target cells without the need for combined modulation of any other NK cell cytotoxicity receptors (e.g. use of an agent to separately bind and/or block inhibitory KIR receptors, or to trigger the activating receptor CD16). Notably, the antibodies induced NK cell cytotoxicity towards tumor cells as pure blocking antibodies that have human Fc domains modified to abolish or decrease binding to CD16 (as well as other Fcγ receptors).

Furthermore, the anti-ILT2 antibodies were able to cause the primary NK cells to lyse HLA-G-expressing tumor target cells that also expressed HLA-E (a HLA class I molecule that inhibits cytotoxicity of NK and CD8 T cells but that is not a ligand of ILT2). The antibodies can therefore also be useful in a cancers characterized by tumor cells that express HLA-E in addition to HLA-G.

Despite the fact that HLA-G binds to other receptors besides ILT2, and previously available blocking anti-ILT2 antibodies generally also bind other ILT2 family members (ILT-1,-4, -5, -6 and combinations thereof), our generation of ILT2-HLA-G interaction-blocking antibodies found that some antibodies bound only to ILT2, and that unlike many antibodies which were effective in neutralizing ILT2 (or inducing NK-mediated cytotoxic activity) only in certain model setting such as highly sorted or engineered NK cells lines made to express ILT2 at high levels, the present antibodies were capable of inducing NK-mediated cytotoxic activity in primary human NK cells (e.g., donor derived NK cells) that have lower levels of expression of ILT2. The difference in potency (when acting on primary NK cells) was not related to binding affinity because the antibodies selected all had comparable strong affinity for ILT2. The most potent antibodies for potentiating primary NK cells were among the group of antibodies that bound to certain epitopes present solely on ILT2 (and not, e.g. on ILT-1, 4,-5 or -6). Thus, there are regions on the protein surface unique to ILT2 among ILT receptors that, when blocked, provide strong potentiation of primary NK cells. Without wishing to be bound by theory, binding ILT2 without binding to ILT6 may have the advantage of providing stronger potentiation of NK and/or CD8 T cell activity because ILT6 is naturally present as a soluble protein which binds HLA class I molecules, thereby acting as a natural inhibitor of inhibitory receptors (other than ILT2) on the surface of the NK and/or T cells.

In view of the complex interaction of HLA-class I molecules as well as 62 M with ILT proteins, a strategy to identify the binding regions on ILT2 was developed that employed proteins made from combinations of ILT2 domain fragments in order to maximize the chances of obtained a correctly configured protein. Results showed that the anti-ILT2 antibodies that showed particularly good potentiation of cytotoxicity in primary human NK cells fell into two different groups. One set bound to the wild type ILT2 polypeptide (and to a range of ILT2 domain proteins) but lost binding to a modified ILT2 protein lacking the D1 domain portion. A second set bound to the wild type ILT2 polypeptide (and to a range of ILT2 domain proteins) but lost binding to a modified ILT2 protein lacking the D4 domain portion. Further point mutation studies within the domains identified by the domain fragment proteins confirmed the aforementioned results.

The antibodies or antigen binding domains of the present disclosure are in one aspect able to enhance effector cell mediated lysis of tumor cells. The antibodies can further neutralize inhibitory signaling of ILT2 in monocytes, macrophages, DC and/or B cells. The antibodies can further be useful in human individuals and/or cells (e.g., NK and/or T cell populations) which express lower levels of inhibitory ILT proteins at their cell surface compared to monocytes, macrophages, DC and/or other cells. The agents that neutralize ILT2 may advantageously both potentiate the activity of cytotoxic NK lymphocytes as well as, via neutralization of ILTs in myeloid cells (DCs), promote the development of an adaptive anti-tumor immune response, notably via the differentiation and/or proliferation of CD8 T cells into cytotoxic CD8 T cells. Furthermore, by binding all functional inhibitory ILT-2 isoforms with comparable binding affinity, the antibodies can further be used across the population of human individuals, e.g., without the need for a diagnostic step prior to treatment to determine which ILT-2 allele(s) are expressed in each individual.

In one embodiment, the antibody, e.g., an antibody or antibody fragment, comprises an immunoglobulin antigen binding domain, optionally hypervariable region, that specifically binds to a human ILT2 protein. The protein neutralizes the inhibitory signaling of the ILT2 protein. In any embodiment, the antigen binding domain (or antibody or other protein that comprises such) can be specified as not binding to a human ILT1 protein. In any embodiment, the antigen binding domain (or antibody or other protein that comprises such) can be specified as not binding to a human ILT4 protein. In any embodiment, the antigen binding domain (or antibody or other protein that comprises such) can be specified as not binding to a human ILT5 protein. In any embodiment, the antigen binding domain (or antibody or other protein that comprises such) can be specified as not binding to a human ILT6 protein. In one embodiment, the antibodies do not bind a soluble human ILT6 protein. In one embodiment, the antibodies do not inhibit the binding of a soluble human ILT6 protein to HLA class I molecules. In any embodiment, the antigen binding domain (or antibody or other protein that comprises such) can be specified as not binding to any one or more of (e.g., lacking binding to each of) ILT-1, ILT-3, ILT-5, ILT-6, ILT-7, ILT-8, ILT-9, ILT-10 and/or ILT-11 proteins; in one embodiment, the antigen binding domain (or antibody or other protein that comprises such) does not bind to any of the human ILT-1, -4, -5 or -6 proteins (e.g., the wild type proteins, the proteins having the amino acid sequences of SEQ ID NOS: 3, 5, 6 and 7 respectively).

In any embodiment herein, any ILT protein (e.g., ILT-2) can be specified to be a protein expressed at the surface of a cell (e.g., a primary or donor cell, an NK cell, a T cell, a DC, a macrophage, a monocyte, a recombinant host cell made to express the protein). In another embodiment herein, any ILT protein (e.g., ILT-2) can be specified to be an isolated, recombinant and/or membrane-bound protein.

Optionally, an antibody can be specified as being an antibody fragment, a full-length antibody, a multi-specific or bi-specific antibody, that specifically binds to a human ILT2 polypeptide and neutralizes the inhibitory activity of the ILT2 polypeptide. Optionally, the ILT2 polypeptide is expressed at the surface of a cell, optionally an effector lymphocyte, an NK cell, a T cell, e.g., a primary NK cell, an NK cell or population of NK cells derived obtained, purified or isolated from a human individual (e.g. without further modification of the cells).

In one aspect, antibodies that specifically bind human ILT2 enhance the cytotoxic activity of NK cells (e.g. as determined by assessing a marker of NK cell cytotoxicity) towards a target cell bearing at its surface a ligand of ILT2 (e.g., a natural ligand; an HLA class I protein, optionally an HLA-A protein, an HLA-B protein, an HLA-F protein, an HLA-G protein). In one embodiment, the NK cells are primary NK cells. Optionally the target cell additionally bears HLA-E protein at its surface. Unlike antibodies that can enhance cytotoxicity only in cells that express high levels of ILT2 (e.g., monocytes, macrophages or ILT2-transfected cells and/or other cells or cell lines (e.g., NK cell lines, T cell lines) that express or are made to express high levels of ILT2 at their cell surface, the antibodies described herein can be functional even in cells that express low levels of ILT2 such as NK cells in a human individual (or from a human donor) The ability to enhance the cytotoxicity of such ILT2 low-expressing NK cells has the advantage of being able to additionally mobilize this population of cells against target cells, e.g., tumor cells, virus-infected cells and/or bacterial cells.

In one embodiment, provided is an antibody or antibody fragment (or a protein that comprises such a fragment) that specifically binds human ILT2 and that enhances and/or restores the cytotoxicity of NK cells (primary NK cells) in a standard 4-hour in vitro cytotoxicity assay in which NK cells that express ILT2 are incubated with target cells that express a ligand (e.g., a natural ligand; an HLA protein, HLA-G protein) of ILT2. Standard NK cell cytotoxicity assays are well-known. In one embodiment the target cells are labeled with ⁵¹Cr prior to addition of NK cells, and then the killing (cytotoxicity) is estimated as proportional to the release of ⁵¹Cr from the cells to the medium. In one embodiment, the antibody or antibody fragment is capable of restoring cytotoxicity of NK cells that express ILT2 to at least the level observed with NK cells that do not express ILT2 (e.g., as determined according to the methods of the Examples herein). In one embodiment, the target cells are K562 cells made to express HLA-G, optionally further K562 cells made to express both HLA-G and HLA-E.

In any aspect herein, NK cells (e.g., primary NK cells) can be specified as being fresh NK cells purified from donors, optionally incubated overnight at 37° C. before use. In any aspect herein, NK cells or primary NK cells can be specified as being ILT2 expressing, e.g., for use in assays the cells can be gated on ILT2 by flow cytometry.

In another embodiment, provided is an antibody or antibody fragment (or a protein that comprises such a fragment) that specifically binds human ILT2 and that neutralizes the inhibitory activity of the ILT2 polypeptide in a human macrophage. In one embodiment, the antibody increases macrophage-mediated ADCC. In one embodiment, the antibody increases activation or signaling in a human macrophage. In one embodiment, the antibody neutralizes the inhibitory activity of the ILT2 polypeptide in the presence of cells bearing natural ligands of ILT2 (e.g., HLA proteins).

In another aspect, the present invention provides function-neutralizing anti-ILT agents (e.g., antibodies) that bind each of the ILT-2 isoform 1 to 6 polypeptides with comparable affinity. Such agents have advantageous pharmacological characteristics. The agents can be used in the same administration regimen (mode of administration, dose and frequency) across the human population, i.e., in individuals expressing different ILT-2 isoforms.

In another aspect of any embodiment herein, the antibodies that bind ILT2 can be characterized as being capable of inhibiting (decreasing) the interactions between ILT2 and a HLA class I ligand(s) thereof, particularly a HLA-A, HLA-B, HLA-F and/or HLA-G protein. In one embodiment, the antibodies that bind ILT2 can be characterized as being capable of inhibiting (decreasing) the interactions between ILT2 and a target cell (e.g., tumor cell) that expresses an HLA ligand(s) of ILT-2, particularly a HLA-A, HLA-B, and/or HLA-G protein.

In any embodiment herein, an antibody can be characterized by a KD for binding affinity of less than 1×10⁻⁸ M, optionally less than 1×10⁻⁹ M, or of about 1×10⁻⁸ M to about 1×10⁻¹⁹ M, or about 1×10⁻⁹ M to about 1×10⁻¹¹ M, for binding to a human a human ILT2 polypeptide. In one embodiment, affinity is monovalent binding affinity. In one embodiment, affinity is bivalent binding affinity.

In any embodiment herein, an antibody can be characterized by a monovalent KD for binding affinity of less than 2 nM, optionally less than 1 nM.

In any embodiment herein, an antibody can be characterized by a 1:1 Binding fit, as determined by SPR. In any embodiment herein, an antibody can be characterized by dissociation or off rate (kd (1/s)) of less than about 1E-2, optionally less than about of less than about 1E-3.

In any embodiment herein, binding affinity can be specified to be monovalent binding as determined by surface plasmon resonance (SPR) screening (such as by analysis with a BIAcore™ SPR analytical device). In any embodiment herein, binding affinity can be specified as being determined by SPR, when anti anti-ILT2 antibodies at 1 μg/mL are captured onto a Protein-A chip and recombinant human ILT2 proteins (e.g., tetrameric ILT2 protein) are injected over captured antibodies.

In one embodiment, the antibodies furthermore do not substantially bind any of human ILT-1, ILT-3, ILT-4, ILT-5, ILT-6, ILT-7, ILT-8, ILT-9, ILT-10 and/or ILT-11 proteins, e.g., amino acid sequences shown in Table 4.

In one embodiment, the antibodies are characterized by a decrease in binding to cells expressing human ILT2 mutant polypeptide having amino acid substitutions at residues 34, 36, 76, 82 and 84 (substitutions E34A, R36A, Y761, A82S, R84L), compared to a wild-type human ILT2 protein, lack of binding to the human ILT-6 polypeptide, and a 1:1 Binding fit and/or dissociation or off rate (kd (1/s)) of less than about 1E-2, optionally less than about of less than about 1E-3, as determined in a SPR monovalent binding affinity assay.

In one embodiment, the antibodies are characterized by a decrease in binding to cells expressing human ILT2 mutant polypeptide having amino acid substitutions at residues F299, Y300, D301, W328, Q378 and K381 (substitutions F299I, Y300R, D301A, W328G, Q378A, K381N), at residues W328, Q330, R347, T349, Y350 and Y355 (substitutions W328G, Q330H, R347A, T349A, Y350S, Y355A) and/or at residues D341, D342, W344,R345 and R347 (substitutions D341A, D342S, W344L, R345A, R347A) compared to a wild-type human ILT2 protein, lack of binding to the human ILT-6 polypeptide, and a 1:1 Binding fit and/or dissociation or off rate (kd (1/s)) of less than about 1E-2, optionally less than about of less than about 1E-3, as determined in a SPR monovalent binding affinity assay.

The affinity can be specified as being determined by SPR, when anti anti-ILT2 antibodies at 1 μg/mL are captured onto a Protein-A chip and recombinant human ILT2 proteins were injected at 5 μg/mL over captured antibodies. In any of the embodiments herein, the anti-ILT antibodies can be characterized by binding to polypeptides expressed on the surface of a cell (e.g., an NK cell, a cell made to express ILT2, e.g., a recombinant CHO host cell made to express ILT2 at its surface, as shown in the Examples), and optionally further wherein the antibody binds with high affinity as determined by flow cytometry. For example, an antibody can be characterized by an EC₅₀, as determined by flow cytometry, of no more than 5 μg/ml, optionally no more than 1 μg/ml, no more than 0.5 μg/ml, no more than 0.2 μg/ml or no more than 0.1 μg/ml, for binding to primary NK cells (e.g., NK cells purified from a biological sample from a human individual or donor), optionally CD56^{dim} NK cells. EC₅₀ can be determined, for example, using 4 or more healthy human donors tested, stainings acquired on a BD FACS Canto II and analyzed using the FlowJo software, and EC₅₀ calculated using a 4-parameter logistic fit.

In another aspect, the present disclosure provides an antibody or antibody fragment (e.g., an antigen binding domain or a protein comprising such), that specifically binds to a human ILT2 polypeptide and is capable of a neutralizing the inhibitory activity of such ILT(s) in immune cells and capable of blocking the interaction of such ILT polypeptide(s) with a HLA ligand thereof. In one embodiment, the ligand is selected from the group consisting of an HLA-A, HLA-B, HLA-F and HLA-G protein. In one embodiment, the antibody or antibody fragment binds to a human ILT2 polypeptide and is capable of a neutralizing the inhibitory activity of such ILT(s) in human immune cells (e.g., NK cells, human primary NK cells; CD56^{dim} NK cells, in human monocytes, in human dendritic cells, in human macrophages, and/or CD8 T cells.

Fragments and derivatives of such antibodies are also provided. In one embodiment, the antibody is an antigen-binding domain (e.g., a single antigen binding domain, a domain made up of a heavy and a light chain variable domain, etc.) capable of binding to the human ILT2 polypeptide. In one embodiment, the antigen-binding domain binds human ILT2 polypeptide. In one embodiment, provided is a protein comprising such an antigen binding domain (e.g., antibody, fusion protein comprising a further non-immunoglobulin domain, Fc-fusion protein, a fusion protein further comprising a cell surface receptor moiety, a multimeric or monomeric protein, a bispecific protein and/or a multispecific protein), or an isolated cell expressing at its surface any of the foregoing proteins. In one embodiment, provided is a nucleic acid encoding such an antigen binding domain.

In one embodiment, the neutralizing anti-ILT antibody of the disclosure relieves the inhibitory activity exerted by ILT2 in immune cells, enhancing the ability of lymphocytes to effectively recognize and/or eliminate cancer cells that express natural ligands of ILT2. The antibodies (or antibody fragments) reduce the ability of cancer cells to escape lysis due to expression of one or the other types of ligand, and they therefore enhance tumor surveillance by the immune system. In one embodiment, provided is an antibody or antibody fragment that specifically binds human ILT2 and relieves the inhibitory activity exerted by ILT2 in human NK cells (e.g., human primary NK cells; CD56^{dim} NK cells), enhancing the ability of the NK cells to effectively recognize and/or eliminate cancer cells that express natural ligands of ILT2 (e.g., one or more HLA proteins).

In one embodiment, the antibody increases cytotoxicity of NK cells, as assessed in a standard in vitro cytotoxicity assay in which NK cells that express ILT2 are purified from human donors and incubated with target cells that express a HLA ligand of ILT2. In one embodiment, increased activation or neutralization of inhibition of cytotoxicity is assessed by increase in a marker of cytotoxicity/cytotoxic potential, e.g., CD107 and/or CD137 expression (mobilization). In one embodiment, increased activation or neutralization of inhibition of cytotoxicity is assessed by increase in ⁵¹Cr release assay.

In one embodiment, provided is an antibody or antibody fragment (as may be incorporated into a protein that comprises such fragment) that binds a human ILT2 polypeptide and is capable of neutralizing the inhibitory activity of an ILT2 polypeptide comprising the amino acid sequence of SEQ ID NOS: 1 or 2. In one embodiment, the antibody or antibody fragment (or a protein that comprises such fragment) is capable of neutralizing the inhibitory activity of said ILT2 polypeptide in primary NK cells that express such ILT2 polypeptide. In one embodiment, the antibody increases cytotoxicity of NK cells, as assessed in a standard in vitro cytotoxicity assay in which NK cells that express the particular ILT2 are purified from human donors and incubated with target cells that express a natural ligand of the ILT2 protein.

In one aspect of any of the embodiments herein, the antibody is a tetrameric (e.g., full length, F(ab)′2 fragment) antibody or an antibody fragment that binds an epitope present on the extracellular domain of a ILT2 in bivalent fashion. For example, the antibody or antibody fragment that binds a ILT in bivalent fashion can comprise two antigen binding domains that each are capable of binding an ILT2 polypeptide. In another aspect of any of the embodiments herein, the antibody binds to a ILT2 in monovalent manner. In one embodiment, the antibody that binds an ILT2 in monovalent manner is a Fab fragment.

In any of the embodiments herein, the antibody that binds to ILT2 is non-depleting towards ILT2-expressing cells.

In one aspect of any of the embodiments herein, the antibody the antibody comprises an Fc domain capable of being bound by the human neonatal Fc receptor (FcRn) but which had decreased (e.g., compared to a native human IgG1) or substantially lacks binding, via its Fc domain, to a human FcγR (e.g., CD16, optionally one or more of, or each of, human CD16A, CD16B, CD32A, CD32B and/or CD64 polypeptides). Optionally the antibody comprises an Fc domain of human IgG1, IgG2, IgG3 of IgG4 isotype comprising an amino acid modification (e.g., one or more substitutions) that decrease the binding affinity of the antibody for one or more of, or each of, human CD16A, CD16B, CD32A, CD32B and/or CD64 polypeptides.

For example, a monoclonal antibody or antibody fragment can be capable of binding to and neutralizing the inhibitory activity a human ILT2 protein, wherein the antibody does not inhibit the binding of a soluble human ILT6 protein to a HLA class I molecule, and wherein the antibody or antibody fragment lacks an Fc domain, comprises a human IgG4 domain or comprises a human Fc domain modified to eliminate binding to a human CD16 polypeptide, optionally further wherein the human Fc domain is modified to reduce binding to human CD16A, CD16B, CD32A, CD32B and CD64 polypeptides.

In any of the embodiments herein, upon binding to a ILT2 on a human lymphocyte, the monoclonal antibody has the ability to enhance or reconstitute lysis of a target human cell bearing an HLA protein ligand of the ILT2 on the target cell surface, and/or has the ability to increase lymphocyte activation (e.g., as determined by an increase in CD107 and/or CD137 expression on a lymphocyte), when said target cell comes into contact with said lymphocyte, e.g., an effector lymphocyte, an NK or a CD8+ T cell from a human individual, e.g., a CD56^{dim} NK cell.

In any of the embodiments herein, the HLA ligand is a natural ligand, e.g., an HLA-A, HLA-B, HLA-F or HLA-G protein.

In any of the embodiments herein, upon binding to a ILT2 on a human lymphocyte (e.g., a primary NK cell), the monoclonal antibody has the ability to reconstitute lysis of a target human cell bearing a HLA ligand of the ILT2 on the target cell surface, when said target cell comes into contact with said lymphocyte.

In one aspect, an antibody binds to the D1 domain of a human ILT2 polypeptide. Domain D1 of human ILT2 polypeptide corresponds to amino acid residues 24 to 121 of SEQ ID NO: 1. In one aspect, the antibody binds to a cell membrane-bound D1 domain polypeptide, optionally a polypeptide consisting of a membrane anchor and one D1 domain), e.g. a polypeptide consisting of the amino acid sequence of SEQ ID NO: 46. In one aspect, an antibody has reduced binding to an ILT2 polypeptide having a mutation at 1, 2, 3, 4, 5, 6, 7 or more residues (or all the residues) in the segment corresponding to residues 24 to 121 of the ILT2 polypeptide of SEQ ID NO: 1.

In one aspect, an antibody binds to a membrane-anchored D1 domain ILT2 protein whose amino acid sequence consists of the sequence shown in SEQ ID NO: 46, but does not bind to any of the membrane-anchored domain ILT2 proteins whose amino acid sequences consist of the sequences shown in SEQ ID NO: 47, 48 or 49.

In one aspect, the anti-ILT2 antibodies bind to a wild-type ILT2 polypeptide (e.g., as expressed at the surface of a cell) but lack binding to an ILT2 polypeptide having a deletion of the segment corresponding to residues 24 to 121 of the ILT2 polypeptide of SEQ ID NO: 1 (e.g., as expressed at the surface of a cell).

In one aspect, an antibody binds to the D4 domain of a human ILT2 polypeptide. Domain D4 of human ILT2 polypeptide corresponds to amino acid residues 322 to 458 of SEQ ID NO: 1. In one aspect, the antibody binds to a cell membrane-bound D4 domain polypeptide, optionally a polypeptide consisting of a membrane anchor and one D4 domain), e.g. a polypeptide consisting of the amino acid sequence of SEQ ID NO: 49. In one aspect, an antibody has reduced binding to an ILT2 polypeptide having a mutation at 1, 2, 3, 4, 5, 6, 7 or more residues (or all the residues) in the segment corresponding to residues 322 to 458 of the ILT2 polypeptide of SEQ ID NO: 1.

In one aspect, an antibody binds to a membrane-anchored D4 domain ILT2 protein whose amino acid sequence consists of the sequence shown in SEQ ID NO: 49, but does not bind to any of the membrane-anchored domain ILT2 proteins whose amino acid sequences consist of the sequences shown in SEQ ID NO: 46, 47 or 48.

In one aspect, the anti-ILT2 antibodies bind to a wild-type ILT2 polypeptide (e.g., as expressed at the surface of a cell) but lack binding to an ILT2 polypeptide having a deletion of the segment corresponding to residues 322 to 458 of the ILT2 polypeptide of SEQ ID NO: 1 (e.g., as expressed at the surface of a cell).

In one aspect, the anti-ILT2 antibodies have reduced binding to an ILT2 polypeptide having a mutation at a residue in the segment corresponding to residues 322 to 458 of the ILT2 polypeptide of SEQ ID NO: 1. In each case, the reduction in binding is compared to a wild-type ILT2 polypeptide of the respective SEQ ID NO 1.

The invention also provides a nucleic acid (or a set of nucleic acids) encoding the human or humanized antibody or antibody fragment having any of the foregoing properties, a vector comprising such a nucleic acid, a cell comprising such a vector, and a method of producing a human anti-ILT antibody, comprising culturing such a cell under conditions suitable for expression of the anti-ILT antibody. The invention also relates to compositions, such as pharmaceutically acceptable compositions and kits, comprising such proteins, nucleic acids, vectors, and/or cells and typically one or more additional ingredients that can be active ingredients or inactive ingredients that promote formulation, delivery, stability, or other characteristics of the composition (e.g., various carriers). The invention further relates various new and useful methods making and using such antibodies, nucleic acids, vectors, cells, organisms, and/or compositions, such as in the modulation of ILT2-mediated biological activities, for example in the treatment of diseases related thereto, notably cancers and infectious disease.

In one embodiment, provided is an antibody that binds ILT2 and that neutralizes the inhibitory activity of human ILT2, for use in the treatment of a cancer (e.g., urothelial carcinoma, a HNSCC, an ovarian cancer, a renal cancer, a lung cancer, an NSCLC) in an individual. Optionally, the antibody is further characterized by any of the properties of the antibodies described herein.

The invention also provides a method of potentiating and/or modulating the activity of immune cells (e.g., NK cells, CD8+ T cells, monocytes, macrophages, DC) activity in a subject in need thereof, for example a method of potentiating NK cell activity by modulating CD56^{dim} NK cells (the major cytotoxic subset), which method comprises administering to the subject an effective amount of any of the foregoing anti-ILT2 antibody compositions.

The antibodies can be used to treat a patient suffering from cancer, for example a cancer characterized by HL-G-expressing tumor cells, optionally a cancer characterized by HLA-G-expressing tumor cells and HLA-E-expressing tumor cells, optionally further a cancer characterized by tumor cells that express both HLA-G and HLA-E. For example, the patient may be suffering from a head and neck squamous cell carcinoma (HNSCC), a lung cancer, optionally an NSCLC, a renal cell carcinoma (e.g. clear cell renal carcinoma, CCRCC), a colorectal carcinoma or an ovarian cancer. In another embodiment, the subject is a patient suffering from an infectious disease, e.g. a viral infection.

The antibodies may be advantageous for use as monotherapy or in combination with other therapeutic agents. The antibodies may be advantageous for use in settings where an individual's anti-immune response is or remains suppressed despite treatment with other immunomodulating agents. In one embodiment, provided is a method of treating a cancer and/or of activating a CD8+ tumor-infiltrating T cell in an individual who has a cancer that is poorly responsive to treatment with an agent that neutralizes the inhibitory activity of PD-1 (e.g. is progressing, has not fully responded or regressed, is non-responsive), the method comprising administering to the individual a therapeutically active amount of an anti-ILT2 antibody.

These aspects are more fully described in, and additional aspects, features, and advantages will be apparent from, the description of the invention provided herein.

### DETAILED DESCRIPTION OF THE DRAWINGS

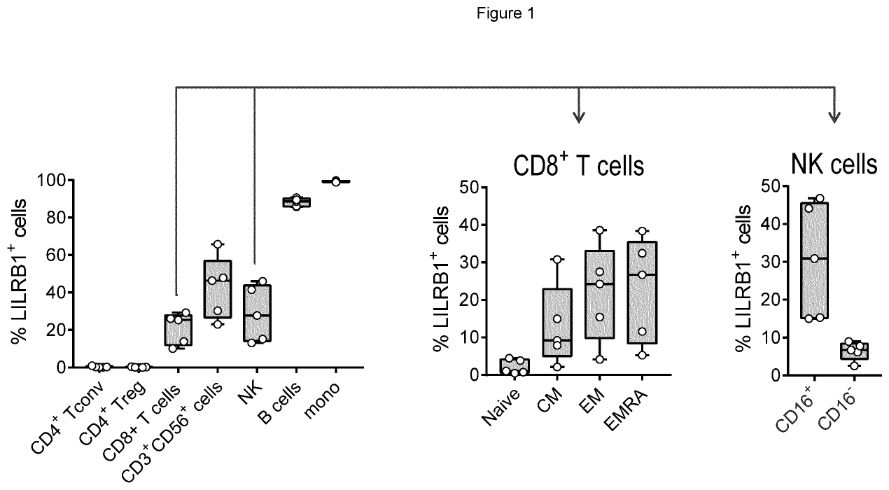

FIG. 1 shows the percent of ILT2 expressing cells in healthy individuals. B lymphocytes and monocytes always express ILT2, conventional CD4 T cells and CD4 Treg cells do not express ILT2, but a significant fraction of CD8 T cells (about 25%), CD3+CD56+ lymphocytes (about 50%) and NK cells (about 30%) expressed ILT2.

FIGS. 2A to 2F shows the percent of ILT2 expressing cells in cancer patients compared to healthy individuals, showing monocytes (FIG. 2A), B cells (FIG. 2B), CD8 T cells (FIG. 2C), CD4γδ T cells (FIG. 2D), CD16⁺ NK cells (FIG. 2E) and CD16⁻ NK cells (FIG. 2F). As can be seen, ILT2 was once again expressed on all monocytes and B cells. However on NK cells and CD8 T cell subsets, ILT2 was expressed more frequently with statistical significance on cells from three types of cancers, HNSCC, NSCLC and RCC, compared to the healthy individuals.

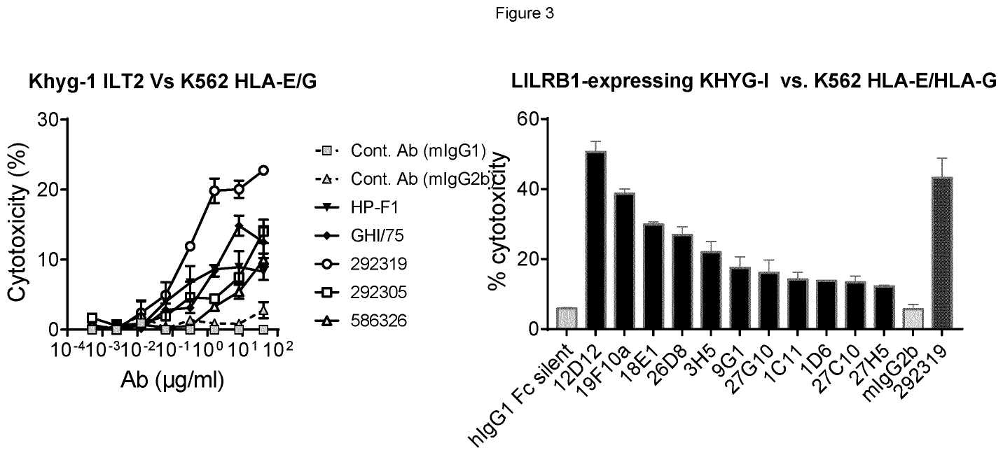

FIG. 3 shows % increase in lysis of K562-HLA-G/HLA-E tumor target cells by ILT2-expressing NK cell lines, in presence of antibodies, compared to isotype controls. Antibodies 12D12, 19F10a and commercial 292319 were significantly more effective than other antibodies in the ability to enhance NK cell cytotoxicity.

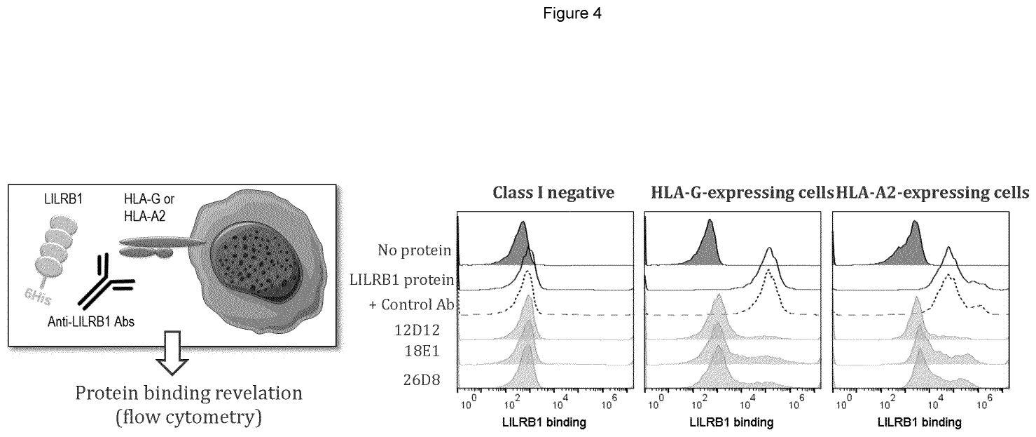

FIG. 4 shows ability of three exemplary anti-ILT2 antibodies to block the interactions between HLA-G or HLA-A2 expressed at the surface of cell lines and recombinant ILT2 protein was assessed by flow cytometry. 12D12, 18E1 and 26D8 each blocked the interaction of ILT2 with each of HLA-G or HLA-A2.

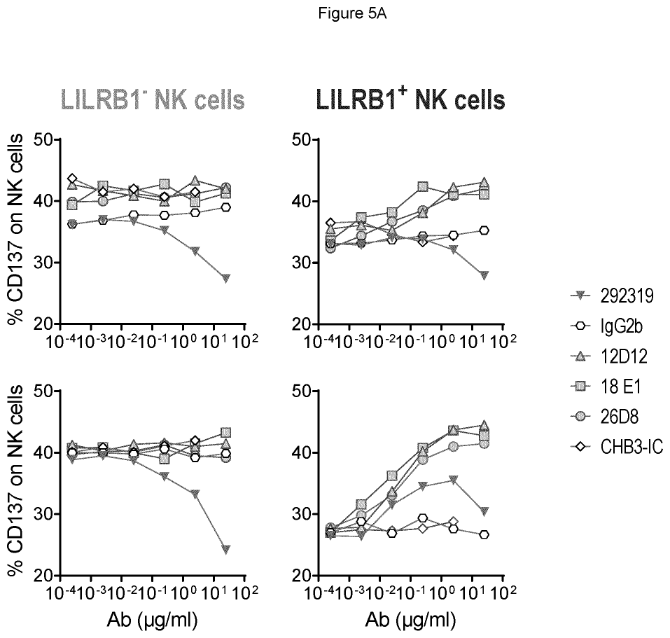

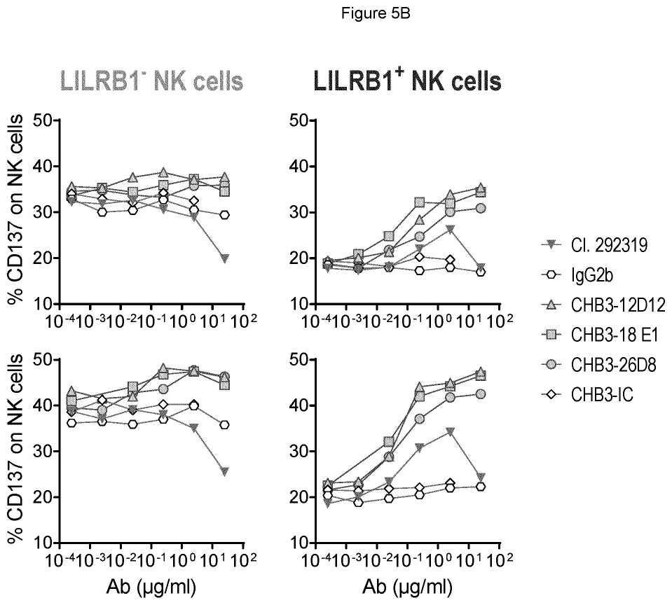

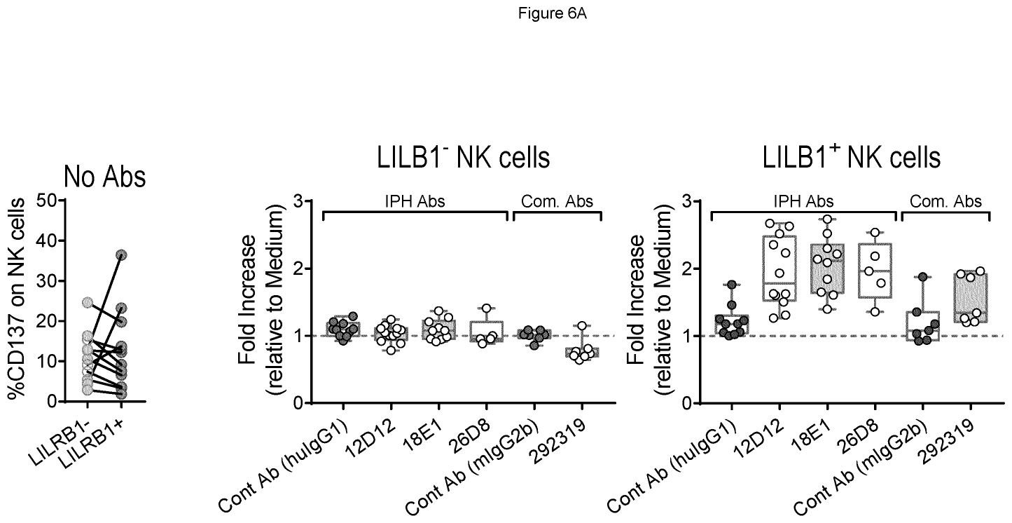

FIG. 5A is a representative figure showing the increase of % of total NK cells expressing CD137 mediated by anti-ILT2 antibodies using primary NK cells (from two human donors) and K562 tumor target cells made to express HLA-E and HLA-G. FIG. 5B is a representative figure showing the increase of % of ILT2-positive (left hand panel) and ILT2-negative (right hand panel) NK cells expressing CD137 mediated anti-ILT2 antibodies using NK cells from two human donors and HLA-A2-expressing B cell line. In each assay with ILT2-positive NK cells, 12D12, 18E1 and 26D8 potentiated NK cell cytotoxicity to a greater extent that antibody 292319. Each of FIGS. 5A and 5B shows the first donor on the top two panels and the second donor on the bottom two panels.

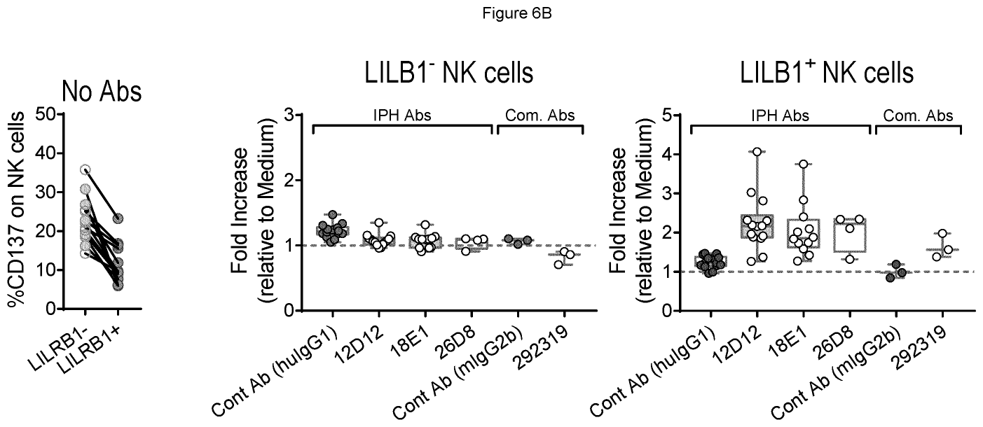

FIGS. 6A and 6B shows the ability of antibodies to enhance cytotoxicity of primary NK cells toward tumor target cells in terms of fold-increase of cytotoxicity marker CD137. FIG. 6A shows the ability of antibodies to enhance NK cell activation in presence of HLA-G-expressing target cells using primary NK cells from 5-12 different donors against HLA-G and HLA-E expressing K562 target cells. FIG. 6A shows the ability of antibodies to enhance NK cell activation in presence of HLA-G-expressing target cells using primary NK cells from 3-14 different donors against HLA-A2 expressing target B cells. In each case 12D12, 18E1 and 26D8 had greater enhancement of NK cytotoxicity.

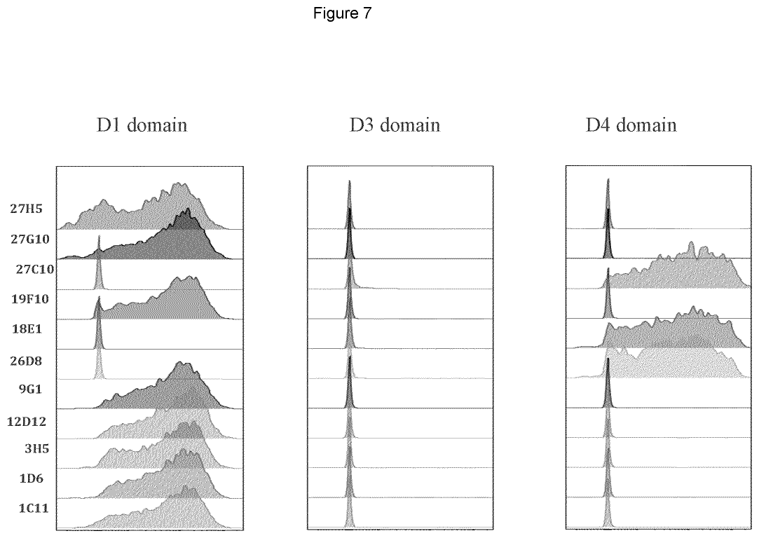

FIG. 7 shows a representative example binding of the antibodies to a subset of the ILT2 domain fragment proteins anchored to the cell surface, as assessed by flow cytometry.

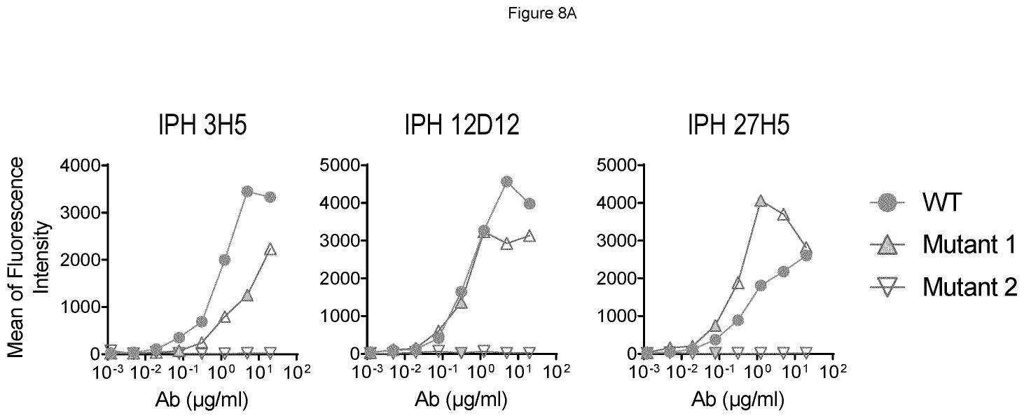

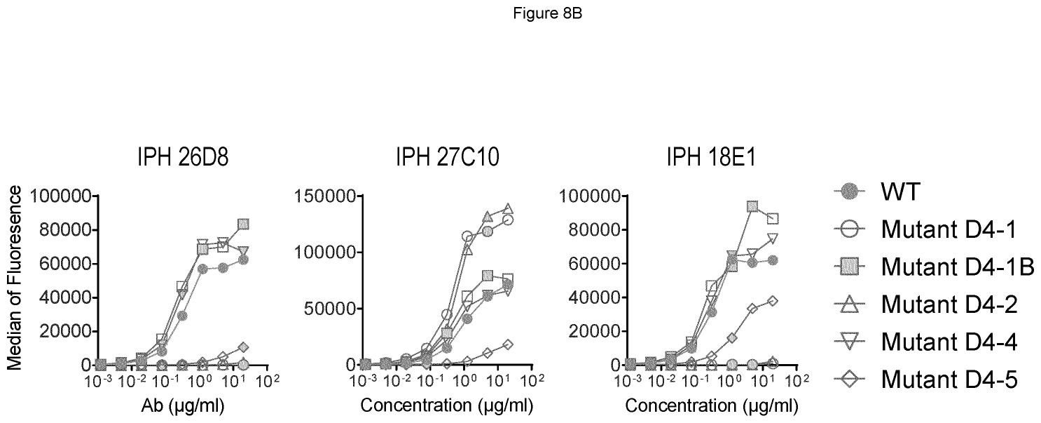

FIG. 8A shows a representative example of titration of antibodies 3H5, 12D12 and 27H5 for binding to mutant ILT2 proteins (mutants 1 and 2) anchored to cells, by flow cytometry, showing the these antibodies lost binding to mutants 2. FIG. 8B shows titration of antibodies 26D8, 18E1 and 27C10 for binding to D4 domain mutants 4-1, 4-1b, 4-2, 4-4 and 4-5 by flow cytometry. Antibodies 26D8 and 18E1 lost binding to mutants 4-1 and 4-2, and 26D8 furthermore lost binding to mutant 4-5, while antibody 18E1 had a decrease in binding (but not complete loss of binding) to mutant 4-5. In contrast, antibody 27C10 which did not potentiate the cytotoxicity of primary NK cells lost binding to mutant 4-5 but retained binding to 4-1 or 4-2.

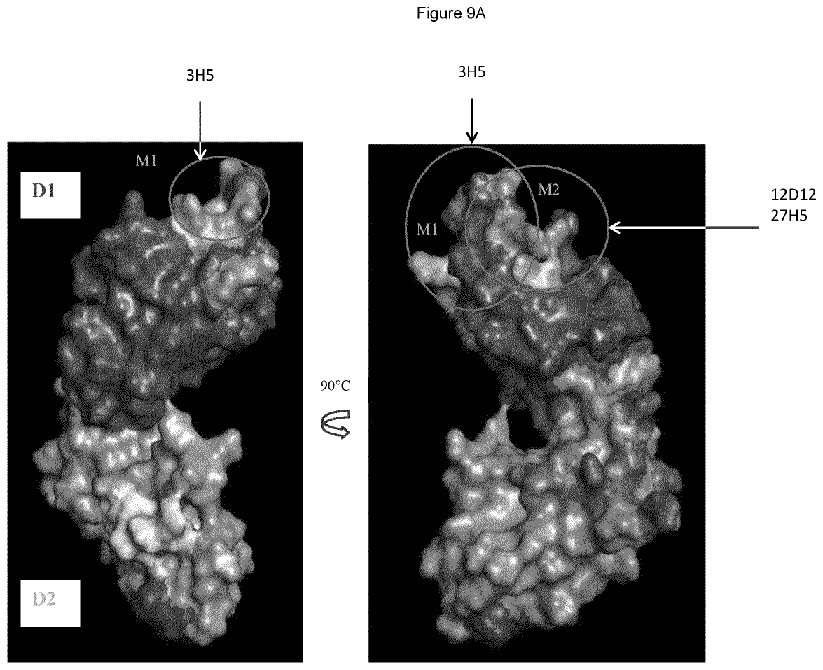

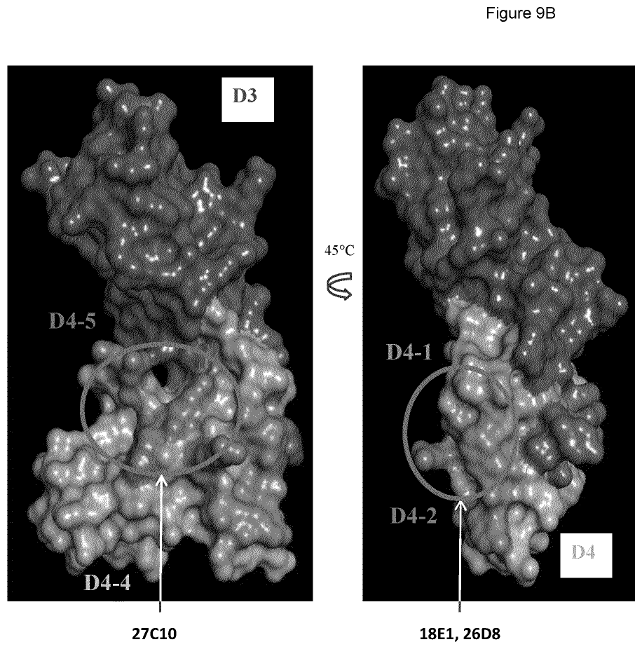

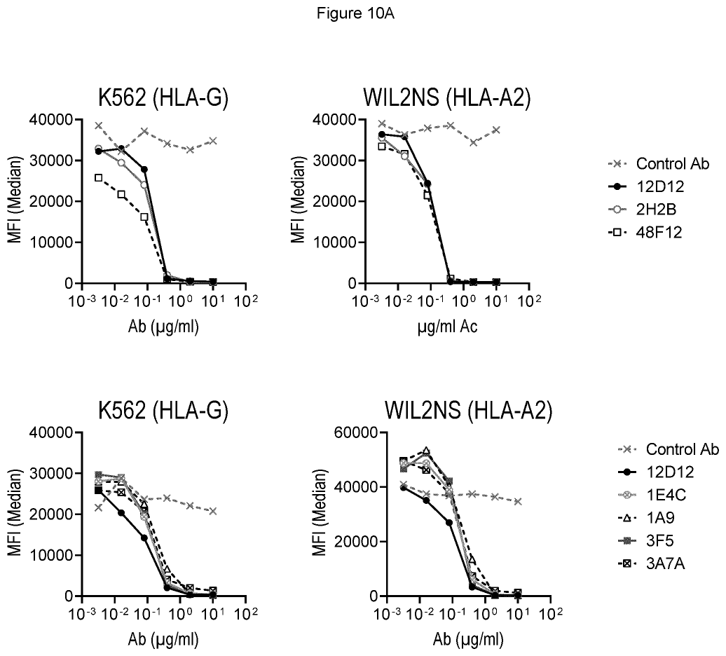

FIG. 9A shows a model representing a portion of the ILT2 molecule that includes domain 1 (top portion, shaded in dark gray) and domain 2 (bottom, shaded in light gray).

FIG. 9B shows a model representing a portion of the ILT2 molecule that includes domain 3 (top portion, shaded in dark gray) and domain 4 (bottom, shaded in light gray).

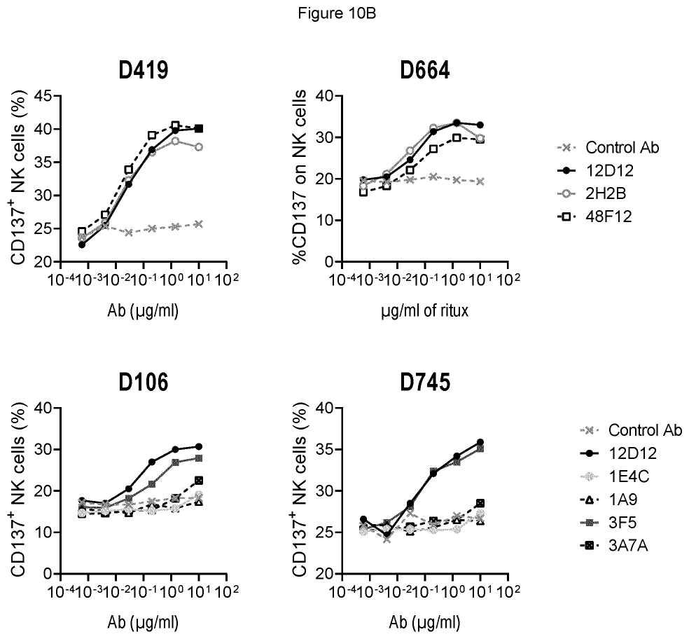

FIG. 10A shows ability of three exemplary anti-ILT2 antibodies to block the interactions between HLA-G or HLA-A2 expressed at the surface of cell lines and recombinant ILT2 protein as assessed by flow cytometry. All antibodies blocked the interactions between HLA-G or HLA-A2, while control antibody did not. FIG. 10B shows the ability of anti-ILT2 antibodies to enhance NK-cell mediated ADCC, determined by assessing cytotoxicity of primary NK cells toward tumor target cells in terms of fold-increase of cytotoxicity marker CD137. While antibodies 12D12, 2H2B, 48F12, and 3F5 were effective in increasing NK cell cytotoxicity, 1A9, 1E4C and 3A7A were not.

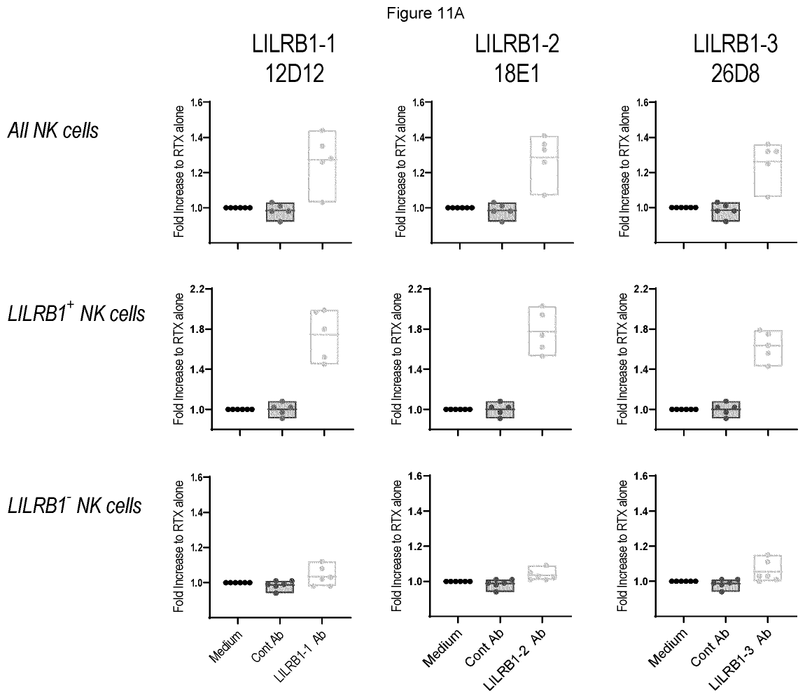

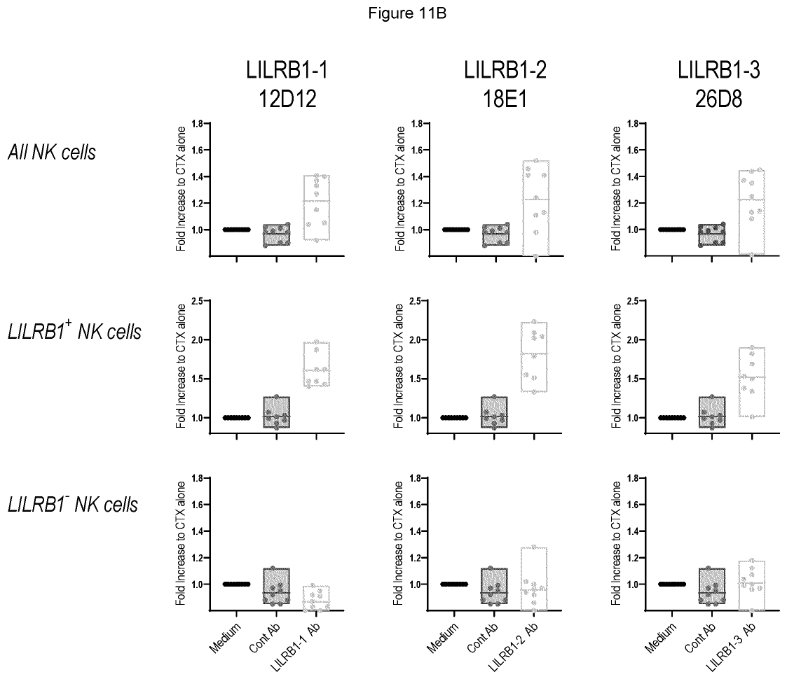

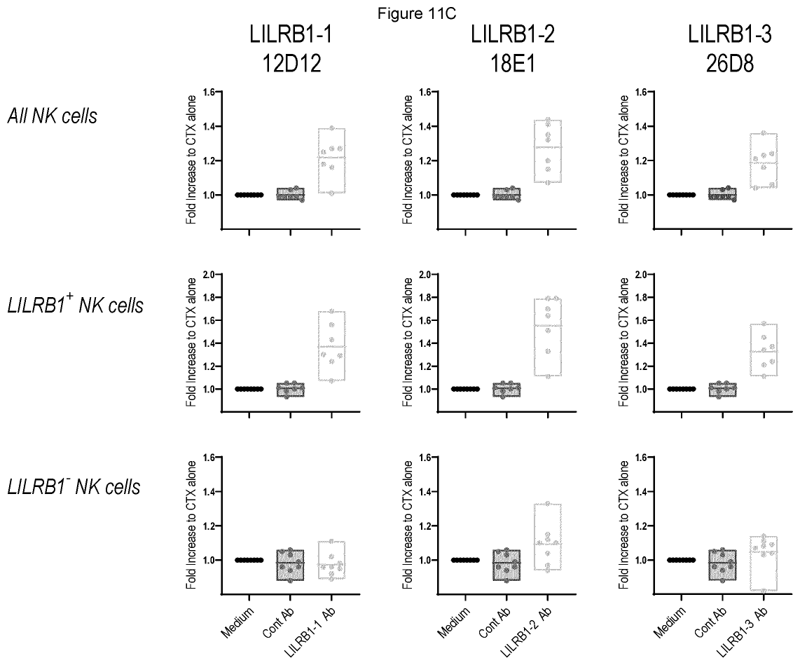

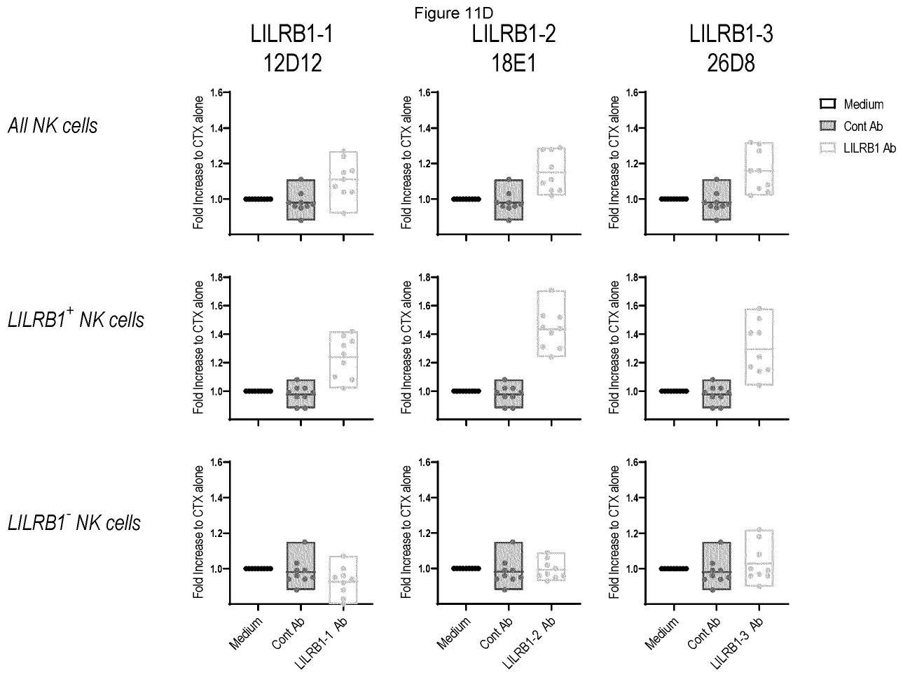

FIGS. 11A, 11B, 11C and 11D shows the ability of anti-ILT2 antibodies 12D12, 18E1 and 26D8 to enhance NK-cell mediated ADCC, determined by assessing cytotoxicity of primary NK cells toward tumor target cells in terms of fold-increase of cytotoxicity marker CD137. FIG. 11A shows the ability of antibodies 12D12, 18E1 and 26D8 to enhance the NK cell activation of primary NK cells mediated by rituximab against tumor target cells, in 3 different human NK cell donors. FIGS. 11B, 11C and 11D show the ability of antibodies 12D12, 18E1 and 26D8 to enhance the NK cell activation of primary NK cells mediated by cetuximab against HN (FIG. 11B), FaDu (FIG. 11C) or Cal27 (FIG. 11D) HNSCC tumor target cells, in each case in 3 different human NK cell donors.

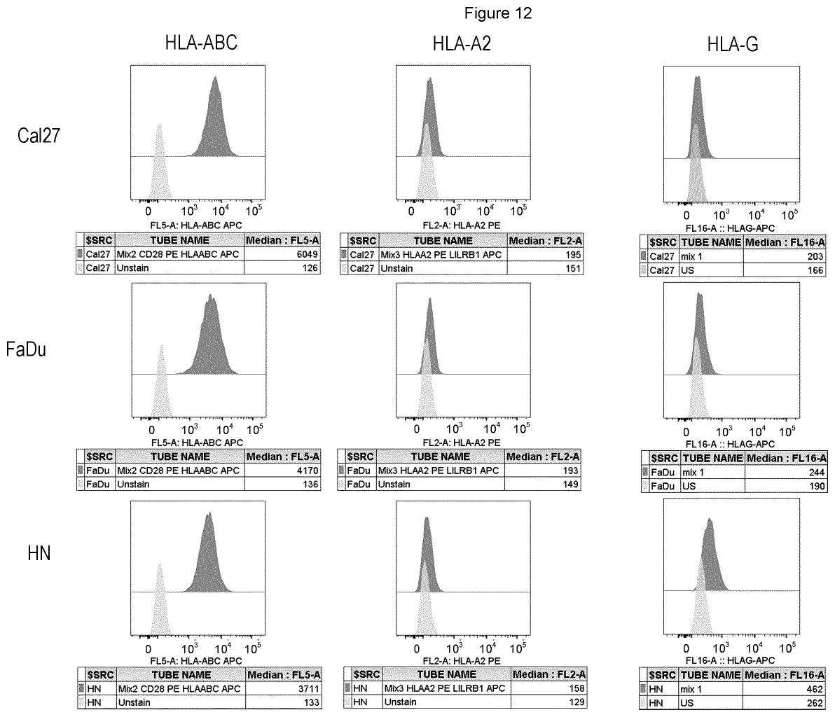

FIG. 12 shows HNSCC tumor cells were found to be consistently negative for HLA-G and HLA-A2, as determined by flow cytometry, but positive for staining with an antibody reactive broadly against HLA-A, B and C alleles.

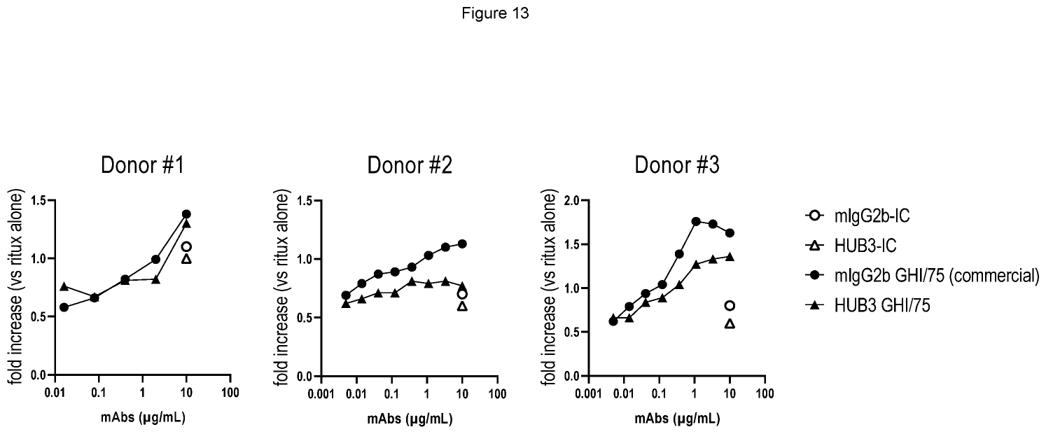

FIG. 13 shows enhancement of ADCP by macrophages towards HLA-A2-expressing B cells by ILT2-blocking antibodies in either mouse IgG2b format that is capable of binding to human Fcγ receptors, or in HUB3 format that is not capable of binding to human Fcγ receptors. Results are shown in terms of fold-increase, in combination with the anti-CD20 antibody rituximab.

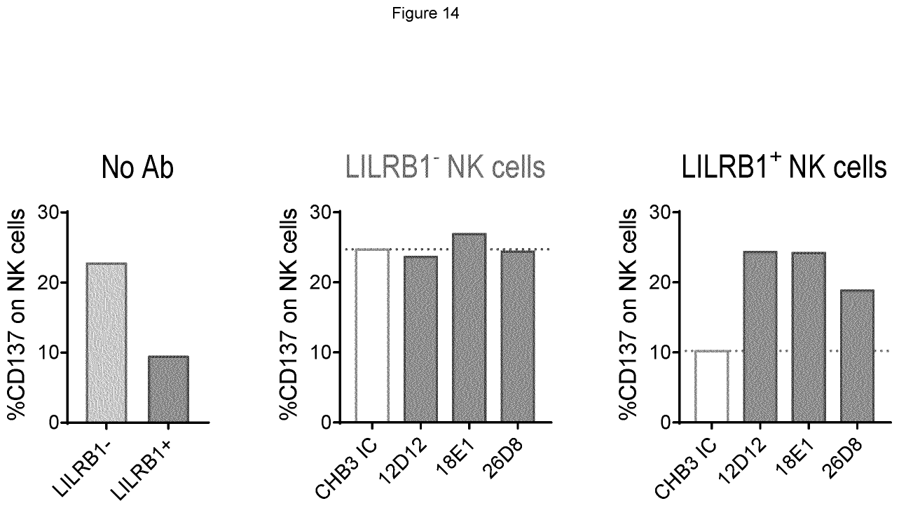

FIG. 14 shows the effect of the anti-ILT2 antibodies on activation of ILT2-positive NK cells and ILT2-negative NK cells from human urothelial cancer patients. Each of the anti-ILT2 antibodies 12D12, 18E1 and 26D8 caused a more than 2-fold increase in NK cell cytotoxicity toward target cells.

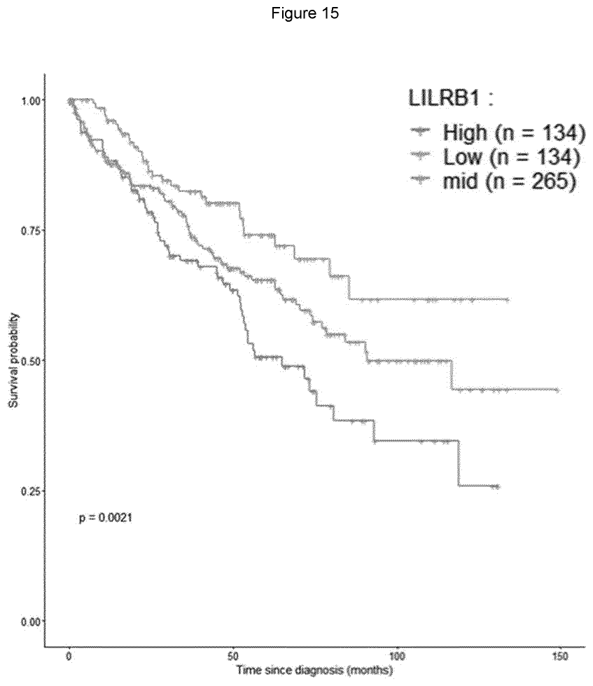

FIG. 15 shows correlation of ILT2 expression levels with survival in CCRCC patients. CCRCC patients were divided in 3 groups (high, mid and low ILT2 gene expression) according to the p-value of the Cox regression (each group must contain at least 10% of patients), and Survival probability curves were drawn for each of the 3 groups. Higher ILT2 correlated with lower probably of survival.

### DETAILED DESCRIPTION

### Definitions

As used in the specification, “a” or “an” may mean one or more.

Where “comprising” is used, this can optionally be replaced by “consisting essentially of” or by “consisting of”.

Human ILT2 is a member of the lymphocyte inhibitory receptor or leukocyte immunoglobulin-(Ig—) like receptor (LIR/LILRs) family. ILT-2 includes 6 isoforms. Uniprot identifier number Q8NHL6, the entire disclosure of which is incorporated herein by reference, is referred to as the canonical sequence, comprises 650 amino acids, and has the following amino acid sequence (including the signal sequence of residues 1-23):

(SEQ ID NO: 1)
MTPILTVLIC LGLSLGPRTH VQAGHLPKPT LWAEPGSVIT
QGSPVTLRCQ GGQETQEYRL YREKKTALWI TRIPQELVKK
GQFPIPSITW EHAGRYRCYY GSDTAGRSES SDPLELVVTG
AYIKPTLSAQ PSPVVNSGGN VILQCDSQVA FDGFSLCKEG
EDEHPQCLNS QPHARGSSRA IFSVGPVSPS RRWWYRCYAY
DSNSPYEWSL PSDLLELLVL GVSKKPSLSV QPGPIVAPEE
TLTLQCGSDA GYNRFVLYKD GERDFLQLAG AQPQAGLSQA
NFTLGPVSRS YGGQYRCYGA HNLSSEWSAP SDPLDILIAG
QFYDRVSLSV QPGPTVASGE NVTLLCQSQG WMQTFLLTKE
GAADDPWRLR STYQSQKYQA EFPMGPVTSA HAGTYRCYGS
QSSKPYLLTH PSDPLELVVS GPSGGPSSPT TGPTSTSGPE
DQPLTPTGSD PQSGLGRHLG VVIGILVAVI LLLLLLLLLF
LILRHRRQGK HWTSTQRKAD FQHPAGAVGP EPTDRGLQWR
SSPAADAQEE NLYAAVKHTQ PEDGVEMDTR SPHDEDPQAV
TYAEVKHSRP RREMASPPSP LSGEFLDTKD RQAEEDRQMD
TEAAASEAPQ DVTYAQLHSL TLRREATEPP PSQEGPSPAV
PSIYATLAIH.

The ILT2 amino acid sequence without the leader sequence is shown below:

(SEQ ID NO: 2)
GHLPKPTLWA EPGSVITQGS PVTLRCQGGQ ETQEYRLYRE
KKTALWITRI PQELVKK GQFPIPSITW EHAGRYRCYY
GSDTAGRSES SDPLELVVTG AYIKPTLSAQ PSPVVNSGGN
VILQCDSQVA FDGFSLCKEG EDEHPQCLNS QPHARGSSRA
IFSVGPVSPS RRWWYRCYAY DSNSPYEWSL PSDLLELLVL
GVSKKPSLSV QPGPIVAPEE TLTLQCGSDA GYNRFVLYKD
GERDFLQLAG AQPQAGLSQA NFTLGPVSRS YGGQYRCYGA
HNLSSEWSAP SDPLDILIAG QFYDRVSLSV QPGPTVASGE
NVTLLCQSQG WMQTFLLTKE GAADDPWRLR STYQSQKYQA
EFPMGPVTSA HAGTYRCYGS QSSKPYLLTH PSDPLELVVS
GPSGGPSSPT TGPTSTSGPE DQPLTPTGSD PQSGLGRHLG
VVIGILVAVI LLLLLLLLLF LILRHRRQGK HWTSTQRKAD
FQHPAGAVGP EPTDRGLQWR SSPAADAQEE NLYAAVKHTQ
PEDGVEMDTR SPHDEDPQAV TYAEVKHSRP RREMASPPSP
LSGEFLDTKD RQAEEDRQMD TEAAASEAPQ DVTYAQLHSL
TLRREATEPP PSQEGPSPAV PSIYATLAIH.

In the context of the present invention, “neutralize” or “neutralize the inhibitory activity of ILT2 refers to a process in which an ILT2 protein is inhibited in its capacity to negatively affect intracellular processes leading to immune cell responses (e.g., cytotoxic responses). For example, neutralization of ILT-2 can be measured for example in a standard NK— or T-cell based cytotoxicity assay, in which the capacity of a therapeutic compound to stimulate killing of HLA positive cells by ILT positive lymphocytes is measured. In one embodiment, an antibody preparation causes at least a 10% augmentation in the cytotoxicity of an ILT-2-restricted lymphocyte, optionally at least a 40% or 50% augmentation in lymphocyte cytotoxicity, or optionally at least a 70% augmentation in NK cytotoxicity, and referring to the cytotoxicity assays described. In one embodiment, an antibody preparation causes at least a 10% augmentation in cytokine release by a ILT-2-restricted lymphocyte, optionally at least a 40% or 50% augmentation in cytokine release, or optionally at least a 70% augmentation in cytokine release, and referring to the cytotoxicity assays described. In one embodiment, an antibody preparation causes at least a 10% augmentation in cell surface expression of a marker of cytotoxicity (e.g., CD107 and/or CD137) by a ILT-2-restricted lymphocyte, optionally at least a 40% or 50% augmentation, or optionally at least a 70% augmentation in cell surface expression of a marker of cytotoxicity (e.g., CD107 and/or CD137).

The ability of an anti-ILT2 antibody to “block” or “inhibit” the binding of an ILT2 molecule to a natural ligand thereof (e.g., an HLA molecule) means that the antibody, in an assay using soluble or cell-surface associated ILT2 and natural ligand (e.g., HLA molecule, for example HLA-A, HLA-B, HLA-F, HLA-G), can detectably reduce the binding of a ILT2 molecule to the ligand (e.g., an HLA molecule) in a dose-dependent fashion, where the ILT2 molecule detectably binds to the ligand (e.g., HLA molecule) in the absence of the antibody.

Whenever within this whole specification “treatment of cancer” or the like is mentioned with reference to anti-ILT2 binding agent (e.g., antibody), there is meant: (a) method of treatment of cancer, said method comprising the step of administering (for at least one treatment) an anti-ILT2 binding agent, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-ILT2 binding agent for the treatment of cancer, or an anti-ILT2 binding agent, for use in said treatment (especially in a human); (c) the use of an anti-ILT2 binding agent for the manufacture of a pharmaceutical preparation for the treatment of cancer, a method of using an anti-ILT2 binding agent for the manufacture of a pharmaceutical preparation for the treatment of cancer, comprising admixing an anti-ILT2 binding agent with a pharmaceutically acceptable carrier, or a pharmaceutical preparation comprising an effective dose of an anti-ILT2 binding agent that is appropriate for the treatment of cancer; or (d) any combination of a), b), and c), in accordance with the subject matter allowable for patenting in a country where this application is filed.

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The terms “antibody” or “immunoglobulin,” as used interchangeably herein, include whole antibodies and any antigen binding fragment or single chains thereof. A typical antibody comprises at least two heavy (H) chains and two light (L) chains interconnected by disulfide bonds. Each heavy chain is comprised of a heavy chain variable region (V_{H}) and a heavy chain constant region. The heavy chain constant region is comprised of three domains, CH1, CH2, and CH3. Each light chain is comprised of a light chain variable region (V_{L}) and a light chain constant region. The light chain constant region is comprised of one domain, CL. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. Several of these are further divided into subclasses or isotypes, such as IgG1, IgG2, IgG3, IgG4, and the like. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., ILT2, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody, it means that the antibody competes with the monoclonal antibody in a binding assay using either recombinant ILT2 molecules or surface expressed ILT2 molecules. For example, if a test antibody reduces the binding of a reference antibody to an ILT2 polypeptide or ILT2-expressing cell in a binding assay, the antibody is said to “compete” respectively with the reference antibody.

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92: 589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “deplete” or “depleting”, with respect to ILT2-expressing cells means a process, method, or compound that results in killing, elimination, lysis or induction of such killing, elimination or lysis, so as to negatively affect the number of such ILT2-expressing cells present in a sample or in a subject. “Non-depleting”, with reference to a process, method, or compound means that the process, method, or compound is not depleting.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7: 13; Lonberg et al. (1994) Nature 368: 856; Taylor et al. (1994) Int Immun 6: 579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348: 552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196: 901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### Production of Antibodies

The anti-ILT2 agents useful for the treatment of disease (e.g., cancer, infectious disease) bind an extra-cellular portion of the human ILT2 protein, optionally without significant or high affinity binding to other ILT family members (e.g., activating ILT and/or other inhibitory ILT), and reduces the inhibitory activity of human ILT2 expressed on the surface of an ILT2 positive immune cell. In one embodiment the agent inhibits the ability of an HLA class I molecule, for example HLA-G and/or HLA-A2 complexed with β₂-microglobulin (B2M), to cause inhibitory signaling by ILT2 in a myeloid cell, a dendritic cell, a macrophage, and/or in a lymphoid cell, optionally an NK cell, a B cell and/or a CD8+ T cell.

In one embodiment, the anti-ILT2 agent described herein can be used to increase the cytotoxicity of NK cells or CD8 T cells in a human or from a human donor toward a target cell that bears ligands of the ILT2 (e.g., a cancer cell, a K562 cell, a WIL2-NS cell, a FaDu cell, a Cal27 cell). The antibodies can be used to enhance NK cell and/or CD8 T cell cytotoxicity, for example to restore the level of cytotoxicity to substantially that observed in NK cells or T cells that do not express at their surface the ILT2 protein.

In one embodiment the agent competes with a class I HLA molecule in binding to an ILT2 molecule, i.e., the agent blocks the interaction between the ILT2 and a HLA class I ligand thereof (e.g. HLA-G and/or HLA-A2, in each case complexed with β₂-microglobulin (B2M)).

In one aspect of the invention, the agent is an antibody selected from a full-length antibody, an antibody fragment, and a synthetic or semi-synthetic antibody-derived molecule.

In one aspect of the invention, the agent is an antibody selected from a fully human antibody, a humanized antibody, and a chimeric antibody.

In one aspect of the invention, the agent is a fragment of an antibody selected from IgA, an IgD, an IgG, an IgE and an IgM antibody.

In one aspect of the invention, the agent is a fragment of an antibody comprising a constant domain selected from IgG1, IgG2, IgG3 and IgG4.

In one aspect of the invention, the agent is an antibody fragment selected from a Fab fragment, a Fab′ fragment, a Fab′-SH fragment, a F(ab)2 fragment, a F(ab′)2 fragment, an Fv fragment, a Heavy chain Ig (a llama or camel Ig), a V_{HH} fragment, a single domain FV, and a single-chain antibody fragment.

In one aspect of the invention, the agent is a synthetic or semisynthetic antibody-derived molecule selected from a scFV, a dsFV, a minibody, a diabody, a triabody, a kappa body, an IgNAR; and a multispecific antibody.

In one aspect, the antibody or antigen binding domains of the disclosure can be characterized as binding to ILT2 with a binding affinity (e.g., KD) at least 100-fold, optionally at least 1000-fold or 10000-fold lower than to a further human ILT, e.g., ILT-1, ILT-3, ILT-4, ILT-5, ILT-6, ILT-7, ILT-8, ILT-9, ILT-10 and/or ILT-11. Affinity can be determined for example by Surface Plasmon Resonance, for binding to recombinant ILT polypeptides (e.g., according to the methods of the Examples herein).

In one aspect of the invention, the antibody is in purified or at least partially purified form. In one aspect of the invention, the antibody is in essentially isolated form.

The antibodies may be produced by a variety of techniques known in the art. Typically, they are produced by immunization of a non-human animal, preferably a mouse, with an immunogen comprising an ILT2 polypeptide, preferably a human ILT2 polypeptide, optionally a polypeptide comprising or consisting essentially of the amino acid sequence of SEQ ID NOS: 1 or 2. The ILT2 polypeptide may comprise the full length sequence of a human ILT2 polypeptide, or a fragment or derivative thereof, typically an immunogenic fragment, i.e., a portion of the polypeptide comprising an epitope exposed on the surface of cells expressing an ILT2 polypeptide. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human ILT2 polypeptide in a lipid membrane, typically at the surface of a cell. In a specific embodiment, the immunogen comprises intact cells, particularly intact human cells, optionally treated or lysed. In another embodiment, the polypeptide is a recombinant ILT2 polypeptide.

The step of immunizing a non-human mammal with an antigen may be carried out in any manner well known in the art for stimulating the production of antibodies in a mouse (see, for example, E. Harlow and D. Lane, Antibodies: A Laboratory Manual, Cold Spring Harbor Laboratory Press, Cold Spring Harbor, N.Y. (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

For monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Murine myeloma lines include, but are not limited to, those derived from MOPC-21 and MPC-11 mouse tumors available from the Salk Institute Cell Distribution Center, San Diego, U.S.A, X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies are then assayed for the production of antibodies that specifically bind to ILT2 polypeptide gene products. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. Typically, the antibodies will also be tested for the ability to bind to ILT2 polypeptides, e.g., ILT2-expressing cells.

Hybridomas that are confirmed to produce a monoclonal antibody can be grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference).

Antibodies can be titrated on ILT2 proteins for the concentration required to achieve maximal binding to a ILT2 polypeptide. “EC50” with respect to binding to a ILT2 polypeptide (or cell expressing such), refers to the efficient concentration of anti-ILT2 antibody which produces 50% of its maximum response or effect with respect to binding to a ILT2 polypeptide (or cell expressing such).

Once antibodies are identified that are capable of binding ILT2 and/or having other desired properties, they will also typically be assessed, using standard methods including those described herein, for their ability to bind to other polypeptides, including other ILT2 polypeptides and/or unrelated polypeptides. Ideally, the antibodies only bind with substantial affinity to ILT2 and do not bind at a significant level to unrelated polypeptides or to other ILT proteins, notably ILT-1, -3, -4, -5, -6, -7, and/or -8). However, it will be appreciated that, as long as the affinity (e.g., KD as determined by SPR) for ILT2 is substantially greater (e.g., 10×, 100×, 1000×, 10,000×, or more) than it is for other ILTs and/or other, unrelated polypeptides), then the antibodies are suitable for use in the present methods.

The anti-ILT2 antibodies can be prepared as non-depleting antibodies such that they have reduced, or substantially lack specific binding to human FCγ receptors. Such antibodies may comprise constant regions of various heavy chains that are known not to bind, or to have low binding affinity for CD16 and optionally further other FCγ receptors. One such example is a human IgG4 constant region which has lowered CD16 binding but retains significant binding to other receptors such as CD64. Alternatively, antibodies with modified Fc domain or antibody fragments that do not comprise constant regions, such as Fab or F(ab′)2 fragments, can be used to avoid Fc receptor binding. Fc receptor binding can be assessed according to methods known in the art, including for example testing binding of an antibody to Fc receptor protein in a BIACORE assay. Any antibody isotype can be used in which the Fc portion is modified to minimize or eliminate binding to Fc receptors (see, e.g., WO03101485, the disclosure of which is herein incorporated by reference). Assays such as, e.g., cell based assays, to assess Fc receptor binding are well known in the art, and are described in, e.g., WO03101485.

The DNA encoding an antibody that binds an epitope present on ILT2 polypeptides is isolated from the hybridoma and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding a monoclonal antibodies can be readily isolated and sequenced using conventional procedures (e. g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

The identification of one or more antibodies that bind(s) to ILT2 polypeptides can be readily determined using any one of a variety of immunological screening assays in which antibody competition can be assessed. Many such assays are routinely practiced and are well known in the art (see, e. g., U.S. Pat. No. 5,660,827, which is incorporated herein by reference). It will be understood that actually determining the epitope to which an antibody described herein binds is not in any way required to identify an antibody that binds to the same or substantially the same epitope as the monoclonal antibody described herein.

Cross-blocking assays can also be used to evaluate whether a test antibody affects the binding of the HLA class I ligand for human ILT2. For example, to determine whether an anti-ILT2 antibody preparation reduces or blocks ILT2 interactions with an HLA class I molecule, the following test can be performed: A dose-range of anti-human ILT2 Fab is co-incubated 30 minutes at room temperature with the human ILT2-Fc at a fixed dose, then added on HLA class 1-ligand expressing cell lines for 1 h. After washing cells two times in staining buffer, a PE-coupled goat anti-mouse IgG Fc fragment secondary antibodies diluted in staining buffer is added to the cells and plates are incubated for 30 additional minutes at 4° C. Cells are washed two times and analyzed on an Accury C6 flow cytometer equipped with an HTFC plate reader. In the absence of test antibodies, the ILT2-Fc binds to the cells. In the presence of an antibody preparation pre-incubated with ILT2-Fc that blocks ILT2-binding to HLA class I, there is a reduced binding of ILT2-Fc to the cells.

In one aspect, the antibodies lack binding to an ILT2 protein modified to lack the D1 domain. In one aspect, the antibodies bind full-length wild-type ILT2 polypeptide but lack binding to an ILT2 protein modified to lack the segment of residues 24 to 121 of the amino acid sequence of SEQ ID NO: 1. In another aspect, the antibodies bind full-length wild-type ILT2 polypeptide but have reduced binding to an ILT2 protein modified to lack the D4 domain. In one aspect, the antibodies bind full-length wild-type ILT2 polypeptide but lack binding to an ILT2 protein modified to lack the segment of residues 322 to 458 of the amino acid sequence of SEQ ID NO: 1.

Binding of anti-ILT2 antibody to cells transfected to express a ILT2 mutant can be measured and compared to the ability of anti-ILT2 antibody to bind cells expressing wild-type ILT2 polypeptide (e.g., SEQ ID NO: 1). A reduction in binding between an anti-ILT2 antibody and a mutant ILT2 polypeptide means that there is a reduction in binding affinity (e.g., as measured by known methods such FACS testing of cells expressing a particular mutant, or by Biacore™ (SPR) testing of binding to mutant polypeptides) and/or a reduction in the total binding capacity of the anti-ILT antibody (e.g., as evidenced by a decrease in Bmax in a plot of anti-ILT2 antibody concentration versus polypeptide concentration). A significant reduction in binding indicates that the mutated residue is directly involved in binding to the anti-ILT2 antibody or is in close proximity to the binding protein when the anti-ILT2 antibody is bound to ILT2.

In some embodiments, a significant reduction in binding means that the binding affinity and/or capacity between an anti-ILT2 antibody and a mutant ILT2 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type ILT2 polypeptide. In certain embodiments, binding is reduced below detectable limits. In some embodiments, a significant reduction in binding is evidenced when binding of an anti-ILT2 antibody to a mutant ILT2 polypeptide is less than 50% (e.g., less than 45%, 40%, 35%, 30%, 25%, 20%, 15% or 10%) of the binding observed between the anti-ILT2 antibody and a wild-type ILT2 polypeptide.

Once an antigen-binding compound having the desired binding for ILT2 is obtained it may be assessed for its ability to inhibit ILT2. For example, if an anti-ILT2 antibody reduces or blocks ILT2 activation induced by a HLA ligand (e.g., as present on a cell), it can increase the cytotoxicity of ILT2-restricted lymphocytes. This can be evaluated by a typical cytotoxicity assay, examples of which are described below.

The ability of an antibody to reduce ILT2-mediated signaling can be tested in a standard 4-hour in vitro cytotoxicity assay using, e.g., NK cells that express ILT2, and target cells that express an HLA ligand of the ILT2. Such NK cells do not efficiently kill targets that express the ligand because ILT2 recognizes the HLA ligand, leading to initiation and propagation of inhibitory signaling that prevents lymphocyte-mediated cytolysis. Such an assay can be carried out using primary NK cells, e.g., fresh NK cells purified from donors, incubated overnight at 37° C. before use. Such an in vitro cytotoxicity assay can be carried out by standard methods that are well known in the art, as described for example in Coligan et al., eds., Current Protocols in Immunology, Greene Publishing Assoc. and Wiley Interscience, N.Y., (1992, 1993). The target cells are labeled with ⁵¹Cr prior to addition of NK cells, and then the killing is estimated as proportional to the release of ⁵¹Cr from the cells to the medium, as a result of killing. The addition of an antibody that prevents ILT2 protein from binding to the HLA class I ligand (e.g. HLA-G) results in prevention of the initiation and propagation of inhibitory signaling via the ILT2 protein. Therefore, addition of such agents results in increases in lymphocyte-mediated killing of the target cells. This step thereby identifies agents that prevent ILT2-mediated negative signaling by, e.g., blocking ligand binding. In a particular ⁵¹Cr-release cytotoxicity assay, ILT2-expressing NK effector-cells can kill HLA ligand-negative target cells, but less well HLA ligand-expressing control cells. Thus, NK effector cells kill less efficiently HLA ligand positive cells due to HLA-induced inhibitory signaling via ILT2. When NK cells are pre-incubated with blocking anti-ILT2 antibodies in such a ⁵¹Cr-release cytotoxicity assay, HLA ligand-expressing cells are more efficiently killed, in an antibody-concentration-dependent fashion.

The inhibitory activity (i.e., cytotoxicity enhancing potential) of an antibody can also be assessed in any of a number of other ways, e.g., by its effect on intracellular free calcium as described, e.g., in Sivori et al., J. Exp. Med. 1997; 186: 1129-1136, the disclosure of which is herein incorporated by reference, or by the effect on markers of NK cell cytotoxicity activation, such as degranulation marker CD107 or CD137 expression. NK or CD8 T cell activity can also be assessed using any cell based cytotoxicity assays, e.g., measuring any other parameter to assess the ability of the antibody to stimulate NK cells to kill target cells such as P815, K562 cells, or appropriate tumor cells as disclosed in Sivori et al., J. Exp. Med. 1997; 186: 1129-1136; Vitale et al., J. Exp. Med. 1998; 187: 2065-2072; Pessino et al. J. Exp. Med. 1998; 188: 953-960; Neri et al. Clin. Diag. Lab. Immun. 2001; 8:1131-1135; Pende et al. J. Exp. Med. 1999; 190:1505-1516, the entire disclosures of each of which are herein incorporated by reference.

In one embodiment, an antibody preparation causes at least a 10% augmentation in the cytotoxicity of an ILT2-restricted lymphocyte, preferably at least a 30%, 40% or 50% augmentation in NK cytotoxicity, or more preferably at least a 60% or 70% augmentation in NK cytotoxicity.

The activity of a cytotoxic lymphocyte can also be addressed using a cytokine-release assay, wherein NK cells are incubated with the antibody to stimulate the cytokine production of the NK cells (for example IFN-γ and TNF-α production). In an exemplary protocol, IFN-γ production from PBMC is assessed by cell surface and intracytoplasmic staining and analysis by flow cytometry after 4 days in culture. Briefly, Brefeldin A (Sigma Aldrich) is added at a final concentration of 5 μg/ml for the last 4 hours of culture. The cells are then incubated with anti-CD3 and anti-CD56 mAb prior to permeabilization (IntraPrep™; Beckman Coulter) and staining with PE-anti-IFN-γ or PE-IgG1 (Pharmingen). GM-CSF and IFN-γ production from polyclonal activated NK cells are measured in supernatants using ELISA (GM-CSF: DuoSet Elisa, R&D Systems, Minneapolis, Minn., IFN-γ: OptEIA set, Pharmingen).

Antibodies can be assessed and/or selected based on binding to human ILT2 without binding to human ILT1, ILT4, ILT5 or ILT6 proteins, e.g. as expressed at the surface of cells. In one aspect, the antibodies bind an antigenic determinant present on human ILT2 expressed at the cell surface. In one embodiment, the determinant is not present on the human ILT6 protein, e.g., as expressed at the surface of a cell; optionally the determinant is not present on any of the human ILT1, ILT4, ILT5 or ILT6 protein, e.g. as expressed at the surface of a cell. the determinant is not present a soluble ILT6 protein, optionally a soluble ILT-6 fragment or a soluble ILT-6 fusion protein such as ILT-6 having an amino acid sequence of Table 4 fused via a linking peptide to a human IgG1 Pro100-Lys330 fragment (as available from R&D Systems, Inc.).

In one embodiment, an anti-ILT2 antibody binds to (and neutralizes the inhibitory activity of) each of the ILT-2 isoform 1, -2, -3, -4, -5 and/or -6 proteins.

In one aspect, provided is a method of producing an antibody which neutralizes the inhibitory activity of ILT2, comprising:
(a) providing a plurality of antibodies that bind an ILT2 protein,
(b) assessing: (i) binding of the antibodies to one or more (or all of) the ILT proteins selected from the group consisting of human ILT-1, -4, -5 and -6 polypeptides, (ii) ability of the antibodies to interfere with the interaction between HLA-G and ILT2 and/or ability of the antibodies to neutralize the inhibitory activity of an ILT2 polypeptide, and (iii) ability of the antibodies to enhance the cytotoxic activity of primary NK cells toward target cells expressing a ligand of ILT-2, for example HLA-G, optionally further HLA-E, and
(c) selecting antibodies (e.g., from those assessed in step (b)) that (i) bind to an ILT2 polypeptide, (ii) that interfere with the interaction between HLA-G and ILT2 and/or neutralize the inhibitory activity of an ILT2 polypeptide, and (iii) that enhance the cytotoxic activity of primary NK cells toward the target cells. Optionally, the method may further comprise the step of assessing the binding of an antibody to a site on an ILT2 polypeptide, and selecting an antibody that binds to domain 1 of an ILT2 polypeptide. In any of the above methods of producing an antibody, the method may further comprise the step of assessing the binding of an antibody to a site on an ILT2 polypeptide, and selecting an antibody that binds to domain 4 of an ILT2 polypeptide. In any of the above methods of producing an antibody, the method may further comprise the step of assessing the binding affinity of an antibody to an ILT2 polypeptide, and selecting an antibody that displays a 1:1 Binding fit and/or dissociation or off rate (kd (1/s)) of less than about 1E-2, optionally less than about of less than about 1E-3, as determined in a SPR monovalent binding affinity assay.

In one aspect, provided is a method of producing an antibody which neutralizes the inhibitory activity of ILT2, comprising:
(a) providing a plurality of antibodies that bind an ILT2 protein,
(b) assessing: (i) binding of the antibodies to one or more (or all of) the ILT proteins selected from the group consisting of human ILT-1, -4, -5 and/or -6 polypeptides, (ii) ability of the antibodies to enhance the cytotoxic activity of primary NK cells toward target cells expressing a ligand of ILT-2, for example HLA-G, optionally further HLA-E, and
(c) selecting antibodies (e.g., from those assessed in step (b)) that (i) bind to an ILT2 polypeptide without binding to human ILT-1, -4, -5 and/or -6 polypeptides, and (ii) enhance the cytotoxic activity of primary NK cells toward the target cells.

In one example, antibodies screening can comprise use of mutant ILT2 polypeptides to characterize and/or orient the selection of antibodies. For example, a method of producing or testing an antibody which binds and neutralizes ILT2, can comprise the steps of:
(a) providing a plurality of antibodies that bind a ILT2 polypeptide,
(b) bringing each of said antibodies into contact with a mutant ILT2 polypeptide comprising a mutation at 1, 2, 3, 4 or 5 or more residues selected from the group consisting of E34, R36, Y76, A82 and R84 (with reference to SEQ ID NO: 2), and assessing binding between the antibody and the mutant ILT2 polypeptide, relative to binding between the antibody and a wild-type ILT2 polypeptide comprising the amino acid sequence of SEQ ID NO: 2, and
(c) selecting an antibody (e.g. for further evaluation, for further processing, production of a quantity of, for use in treatment) that has reduced binding to the mutant ILT2 polypeptide, relative to binding between the antibody and a wild-type ILT2 polypeptide comprising the amino acid sequence of SEQ ID NO: 2. The method can optionally further comprise a step (d) comprising assessing the ability of the antibodies to enhance the cytotoxic activity of NK cells toward target cells expressing a ligand of ILT-2 and selecting an antibody that enhances the cytotoxic activity of NK cells toward the target cells.

In one example, antibodies screening can comprise use of mutant ILT2 polypeptides to characterize and/or orient the selection of antibodies. For example, a method of producing or testing an antibody which binds and neutralizes ILT2, can comprise the steps of:
(a) providing a plurality of antibodies that bind a ILT2 polypeptide,
(b) bringing each of said antibodies into contact with a mutant ILT2 polypeptide comprising a mutation at 1, 2, 3, 4, 5, 6, 7 or more residues selected from the group consisting of 299, 300, 301, 328, 330, 347, 349, 350, 355, 378 and 381 (with reference to SEQ ID NO: 2), and assessing binding between the antibody and the mutant ILT2 polypeptide, relative to binding between the antibody and a wild-type ILT2 polypeptide comprising the amino acid sequence of SEQ ID NO: 2, and
(c) selecting an antibody (e.g. for further evaluation, for further processing, production of a quantity of, for use in treatment) that has reduced binding to the mutant ILT2 polypeptide, relative to binding between the antibody and a wild-type ILT2 polypeptide comprising the amino acid sequence of SEQ ID NO: 2.

The method can optionally further comprise a step (d) comprising assessing the ability of the antibodies to enhance the cytotoxic activity of NK cells toward target cells expressing a ligand of ILT-2 and selecting an antibody that enhances the cytotoxic activity of NK cells toward the target cells. In one embodiment, step (b) comprises bringing each of said antibodies into contact with a mutant ILT2 polypeptide comprising a mutation at 1, 2, 3, 4, 5, or 6 residues selected from the group consisting of 299, 300, 301, 328, 378 and 381. In one embodiment, step (b) comprises bringing each of said antibodies into contact with a mutant ILT2 polypeptide comprising a mutation at 1, 2, 3, 4, 5, or 6 residues selected from the group consisting of 328, 330, 347, 349, 350 and 355.

In any of the above methods of producing an antibody, the method may further comprise the step of assessing the binding affinity of an antibody to an ILT2 polypeptide, and selecting an antibody that is characterized by dissociation or off rate (kd (1/s)) of less than about 1E-2, as determined in a binding assay by SPR. The antibodies selected can then be further produced (e.g. in a recombinant host cell), further evaluated for biological activity (e.g. ability to potentiate the activity of immune cells, primary NK cells, etc.), and/or designated for use or used in the treatment of disease (e.g. cancer).

Advantageously, antibodies can optionally be identified and selected based on binding to the same region or epitope on the surface of the ILT2 polypeptide as any of the antibodies described herein, e.g., 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 (e.g. an epitope- or binding region-directed screen). In one aspect, the antibodies bind substantially the same epitope as any of antibodies 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. In one embodiment, the antibodies bind to an epitope of ILT2 that at least partially overlaps with, or includes at least one residue in, the epitope bound by antibody 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. The residues bound by the antibody can be specified as being present on the surface of the ILT2 polypeptide, e.g., on an ILT2 polypeptide expressed on the surface of a cell.

Binding of anti-ILT2 antibody to a particular site on ILT2 can be assessed by measuring binding of an anti-ILT2 antibody to cells transfected with ILT2 mutants, as compared to the ability of anti-ILT2 antibody to bind wild-type ILT2 polypeptide (e.g., SEQ ID NO: 1). A reduction in binding between an anti-ILT2 antibody and a mutant ILT2 polypeptide (e.g., a mutant of Table 6) means that there is a reduction in binding affinity (e.g., as measured by known methods such FACS testing of cells expressing a particular mutant, or by Biacore testing of binding to mutant polypeptides) and/or a reduction in the total binding capacity of the anti-ILT2 antibody (e.g., as evidenced by a decrease in Bmax in a plot of anti-ILT2 antibody concentration versus polypeptide concentration). A significant reduction in binding indicates that the mutated residue is directly involved in binding to the anti-ILT2 antibody or is in close proximity to the binding protein when the anti-ILT2 antibody is bound to ILT2.

In some embodiments, a significant reduction in binding means that the binding affinity and/or capacity between an anti-ILT2 antibody and a mutant ILT2 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type ILT2 polypeptide. In certain embodiments, binding is reduced below detectable limits. In some embodiments, a significant reduction in binding is evidenced when binding of an anti-ILT2 antibody to a mutant ILT2 polypeptide is less than 50% (e.g., less than 45%, 40%, 35%, 30%, 25%, 20%, 15% or 10%) of the binding observed between the anti-ILT2 antibody and a wild-type ILT2 polypeptide.

In some embodiments, anti-ILT2 antibodies are provided that exhibit significantly lower binding for a mutant ILT2 polypeptide in which a residue in a segment comprising an amino acid residue bound by antibody 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 is substituted with a different amino acid, compared to a binding to a wild-type ILT2 polypeptide not comprising such substitution(s) (e.g. a polypeptide of SEQ ID NO: 1).

In some embodiments, anti-ILT2 antibodies (e.g., other than 12D12, 26D8 or 18E1) are provided that bind the epitope on ILT2 bound by antibody 12D12, 26D8 or 18E1.

In any embodiment herein, an antibody can be characterized as an antibody other than GHI/75, 292319, HP—F1, 586326 and 292305 (or an antibody sharing the CDRs thereof).

In one aspect, an anti-ILT2 antibody binds an epitope positioned on or within the D1 domain (domain 1) of the human ILT2 protein. In one aspect, an anti-ILT2 antibody competes with antibody 12D12 for binding to an epitope on the D1 domain (domain 1) of the human ILT2 protein.

The D1 domain can be defined as corresponding or having the amino acid sequence as follows:

(SEQ ID NO: 55)
GHLPKPTLWAEPGSVITQGSPVTLRCQGGQETQEYRLYREKKTALWITRI
PQELVKKGQFPIPSITWEHAGRYRCYYGSDTAGRSESSDPLELVVTGA.

In one aspect, the anti-ILT2 antibody has reduced binding, optionally loss of binding, to an ILT2 polypeptide having a mutation at a residue selected from the group consisting of: E34, R36, Y76, A82 and R84 (with reference to SEQ ID NO: 2); optionally, the mutant ILT2 polypeptide has the mutations: E34A, R36A, Y761, A82S, R84L. In one embodiment, an antibody furthermore has reduced binding to a mutant ILT2 polypeptide comprising a mutation at one or more (or all of) residues selected from the group consisting of G29, Q30, Q33, T32 and D80 (with reference to SEQ ID NO: 2), optionally, the mutant ILT2 polypeptide has the mutations: G29S, Q30L, Q33A, T32A, D80H. In one aspect, the anti-ILT2 antibody has reduced binding, optionally loss of binding, to an ILT2 polypeptide having the mutations: G29S, Q30L, Q33A, T32A, E34A, R36A, Y761, A82S, D80H and R84L. In each case, a decrease or loss of binding can be specified as being relative to binding between the antibody and a wild-type ILT2 polypeptide comprising the amino acid sequence of SEQ ID NO: 2.

In one aspect, the anti-ILT2 antibody binds an epitope on ILT2 comprising an amino acid residue (e.g., one, two, three, four or five of the residues) selected from the group consisting of E34, R36, Y76, A82 and R84 (with reference to SEQ ID NO: 2). In one aspect, the anti-ILT2 antibody binds an epitope on ILT2 comprising an amino acid residue (e.g., one, two, three, four or five of the residues) selected from the group consisting of G29, Q30, Q33, T32 and D80 (with reference to SEQ ID NO: 2). In one aspect, the anti-ILT2 antibody binds an epitope on ILT2 comprising: (i) an amino acid residue (e.g., one, two, three, four or five of the residues) selected from the group consisting of E34, R36, Y76, A82 and R84, and (ii) an amino acid residue (e.g., one, two, three, four or five of the residues) selected from the group consisting of G29, Q30, Q33, T32 and D80. In one aspect, the anti-ILT2 antibody binds an epitope on ILT2 comprising an amino acid residue (e.g., one, two, three, four or five of the residues) selected from the group consisting of G29, Q30, Q33, T32, E34, R36, Y76, A82, D80 and R84.

In one aspect, an anti-ILT2 antibody binds an epitope positioned on or within the D4 domain (domain 4) of the human ILT2 protein. In one aspect, an anti-ILT2 antibody competes with antibody 26D8 and/or 18E1 for binding to an epitope on the D4 domain (domain 4) of the human ILT2 protein.

The D4 domain can be defined as corresponding or having the amino acid sequence as follows:

(SEQ ID NO: 56)
FYDRVSLSVQPGPTVASGENVTLLCQSQGWMQTFLLTKEGAADDPWRLRS
TYQSQKYQAEFPMGPVTSAHAGTYRCYGSQSSKPYLLTHPSDPLELVVSG
PSGGPSSPTTGPTSTSGPEDQPLTPTGSDPQSGLGRH.

In one aspect, the anti-ILT2 antibody has reduced binding, optionally loss of binding, to an ILT2 polypeptide having a mutation at a residue selected from the group consisting of: F299, Y300, D301, W328, Q378 and K381 (with reference to SEQ ID NO: 2); optionally, the mutant ILT2 polypeptide has the mutations: F299I, Y300R, D301A, W328G, Q378A, K381N. In one embodiment, an antibody furthermore has reduced binding to a mutant ILT2 polypeptide comprising a mutation at one or more (or all of) residues selected from the group consisting of W328, Q330, R347, T349, Y350 and Y355 (with reference to SEQ ID NO: 2), optionally, the mutant ILT2 polypeptide has the mutations: W328G, Q330H, R347A, T349A, Y350S, Y355A. In one embodiment, an antibody furthermore has reduced binding to a mutant ILT2 polypeptide comprising a mutation at one or more (or all of) residues selected from the group consisting of D341, D342, W344, R345 and R347 (with reference to SEQ ID NO: 2), optionally, the mutant ILT2 polypeptide has the mutations: D341A, D342S, W344L, R345A, R347A. In one embodiment, an antibody has reduced binding to a mutant ILT2 polypeptide having the mutations: F299I, Y300R, D301A, W328G, Q330H, R347A, T349A, Y350S, Y355A, Q378A and K381N. In one embodiment, an antibody has reduced binding to a mutant ILT2 polypeptide having the mutations F299I, Y300R, D301A, W328G, D341, D342, W344, R345, R347, Q378A and K381N. In one embodiment, an antibody has reduced binding to a mutant ILT2 polypeptide having the mutations: F299I, Y300R, D301A, W328G, Q330H, D341A, D342S, W344L, R345A, R347A, T349A, Y350S, Y355A, Q378A and K381N. In each case, a decrease or loss of binding can be specified as being relative to binding between the antibody and a wild-type ILT2 polypeptide comprising the amino acid sequence of SEQ ID NO: 2.

In one aspect, the anti-ILT2 antibody binds an epitope on ILT2 comprising an amino acid residue (e.g., one, two, three, four or five of the residues) selected from the group consisting of F299, Y300, D301, W328, Q378 and K381 (with reference to SEQ ID NO: 2). In one aspect, the anti-ILT2 antibody binds an epitope on ILT2 comprising an amino acid residue (e.g., one, two, three, four or five of the residues) selected from the group consisting of W328, Q330, R347, T349, Y350 and Y355 (with reference to SEQ ID NO: 2). In one aspect, the anti-ILT2 antibody binds an epitope on ILT2 comprising an amino acid residue (e.g., one, two, three, four or five of the residues) selected from the group consisting of D341, D342, W344, R345 and R347 (with reference to SEQ ID NO: 2).

In one aspect, the anti-ILT2 antibody binds an epitope on ILT2 comprising an amino acid residue (e.g., one, two, three, four or five of the residues) selected from the group consisting of: F299, Y300, D301, W328, Q330, D341, D342, W344, R345, R347, T349, Y350, Y355, Q378 and K381.

In one aspect, the anti-ILT2 antibody binds an epitope on ILT2 comprising: (i) an amino acid residue (e.g., one, two, three, four or five of the residues) selected from the group consisting of F299, Y300, D301, W328, Q378 and K381, and (ii) an amino acid residue (e.g., one, two, three, four or five of the residues) selected from the group consisting of Q330, R347, T349, Y350 and Y355. In one aspect, the anti-ILT2 antibody binds an epitope on ILT2 comprising: (i) an amino acid residue (e.g., one, two, three, four or five of the residues) selected from the group consisting of F299, Y300, D301, W328, Q378 and K381, (ii) an amino acid residue (e.g., one, two, three, four or five of the residues) selected from the group consisting of Q330, R347, T349, Y350 and Y355, and (iii) an amino acid residue (e.g., one, two, three, four or five of the residues) selected from the group consisting of D341, D342, W344, R345 and R347.

### Antibody CDR Sequences

The amino acid sequence of the heavy chain variable region of antibody 26D8 is listed as SEQ ID NO: 12 (see also Table A), the amino acid sequence of the light chain variable region is listed as SEQ ID NO: 13 (see also Table A). In a specific embodiment, provided is an antibody that binds essentially the same epitope or determinant as monoclonal antibodies 26D8; optionally the antibody comprises the hypervariable region of antibody 26D8. In any of the embodiments herein, antibody 26D8 can be characterized by the amino acid sequences and/or nucleic acid sequences encoding it. In one embodiment, the monoclonal antibody comprises the Fab or F(ab′)₂ portion of 26D8. Also provided is an antibody or antibody fragment that comprises the heavy chain variable region of 26D8. According to one embodiment, the antibody or antibody fragment comprises the three CDRs of the heavy chain variable region of 26D8. Also provided is an antibody or antibody fragment that further comprises the variable light chain variable region of 26D8 or one, two or three of the CDRs of the light chain variable region of 26D8. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody, wherein the antibody or antibody fragment comprises: a HCDR1 region of 26D8 comprising an amino acid sequence EHTIH (SEQ ID NO: 14), or a sequence of at least 3, 4 or 5 contiguous amino acids thereof, optionally wherein one or more of these amino acids may be substituted by a different amino acid; a HCDR2 region of 26D8 comprising an amino acid sequence WFYPGSGSMKYNEKFKD (SEQ ID NO: 15), or a sequence of at least 4, 5, 6, 7, 8, 9 or 10 contiguous amino acids thereof, optionally wherein one or more of these amino acids may be substituted by a different amino acid; a HCDR3 region of 26D8 comprising an amino acid sequence HTNWDFDY (SEQ ID NO: 16), or a sequence of at least 4, 5, 6, 7, 8, 9 or 10 contiguous amino acids thereof, optionally wherein one or more of these amino acids may be substituted by a different amino acid; a LCDR1 region of 26D8 comprising an amino acid sequence KASQSVDYGGDSYMN (SEQ ID NO: 17), or a sequence of at least 4, 5, 6, 7, 8, 9 or 10 contiguous amino acids thereof, optionally wherein one or more of these amino acids may be substituted by a different amino acid; a LCDR2 region of 26D8 comprising an amino acid sequence AASNLES (SEQ ID NO: 18), or a sequence of at least 4, 5, or 6 contiguous amino acids thereof, optionally wherein one or more of these amino acids may be substituted by a different amino acid; a LCDR3 region of 26D8 comprising an amino acid sequence QQSNEEPWT (SEQ ID NO: 19), or a sequence of at least 4, 5, 6, 7, or 8 contiguous amino acids thereof, optionally wherein one or more of these amino acids may be deleted or substituted by a different amino acid.

The amino acid sequence of the heavy chain variable region of antibody 18E1 is listed as SEQ ID NO: 20 (see also Table A), the amino acid sequence of the light chain variable region is listed as SEQ ID NO: 21 (see also Table A). In a specific embodiment, provided is an antibody that binds essentially the same epitope or determinant as monoclonal antibodies 18E1; optionally the antibody comprises the hypervariable region of antibody 18E1. In any of the embodiments herein, antibody 18E1 can be characterized by the amino acid sequences and/or nucleic acid sequences encoding it. In one embodiment, the monoclonal antibody comprises the Fab or F(ab′)₂ portion of 18E1. Also provided is an antibody or antibody fragment that comprises the heavy chain variable region of 18E1. According to one embodiment, the antibody or antibody fragment comprises the three CDRs of the heavy chain variable region of 18E1. Also provided is an antibody or antibody fragment that further comprises the variable light chain variable region of 18E1 or one, two or three of the CDRs of the light chain variable region of 18E1. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody, wherein the antibody or antibody fragment comprises: a HCDR1 region of 18E1 comprising an amino acid sequence AHTIH (SEQ ID NO: 22), or a sequence of at least 3 or 4 contiguous amino acids thereof, optionally wherein one or more of these amino acids may be substituted by a different amino acid; a HCDR2 region of 18E1 comprising an amino acid sequence WLYPGSGSIKYNEKFKD (SEQ ID NO: 23), or a sequence of at least 4, 5, 6, 7, 8, 9 or 10 contiguous amino acids thereof, optionally wherein one or more of these amino acids may be substituted by a different amino acid; a HCDR3 region of 18E1 comprising an amino acid sequence HTNWDFDY (SEQ ID NO: 24), or a sequence of at least 4, 5, 6, 7, 8, 9 or 10 contiguous amino acids thereof, optionally wherein one or more of these amino acids may be substituted by a different amino acid; a LCDR1 region of 18E1 comprising an amino acid sequence KASQSVDYGGASYMN (SEQ ID NO: 25), or a sequence of at least 4, 5, 6, 7, 8, 9 or 10 contiguous amino acids thereof, optionally wherein one or more of these amino acids may be substituted by a different amino acid; a LCDR2 region of 18E1 comprising an amino acid sequence AASNLES (SEQ ID NO: 26), or a sequence of at least 4, 5 or 6 10 contiguous amino acids thereof, optionally wherein one or more of these amino acids may be substituted by a different amino acid; a LCDR3 region of 18E1 comprising an amino acid sequence QQSNEEPWT (SEQ ID NO: 27), or a sequence of at least 4, 5, 6 or 7 contiguous amino acids thereof, optionally wherein one or more of these amino acids may be deleted or substituted by a different amino acid.

The amino acid sequence of the heavy chain variable region of antibody 12D12 is listed as SEQ ID NO: 28 (see also Table A), the amino acid sequence of the light chain variable region is listed as SEQ ID NO: 29 (see also Table A). In a specific embodiment, provided is an antibody that binds essentially the same epitope or determinant as monoclonal antibodies 12D12; optionally the antibody comprises the hypervariable region of antibody 12D12. In any of the embodiments herein, antibody 12D12 can be characterized by the amino acid sequences and/or nucleic acid sequences encoding it. In one embodiment, the monoclonal antibody comprises the Fab or F(ab′)₂ portion of 12D12. Also provided is an antibody or antibody fragment that comprises the heavy chain variable region of 12D12. According to one embodiment, the antibody or antibody fragment comprises the three CDRs of the heavy chain variable region of 12D12. Also provided is an antibody or antibody fragment that further comprises the variable light chain variable region of 12D12 or one, two or three of the CDRs of the light chain variable region of 12D12. The HCDR1, 2, 3 and LCDR1, 2, 3 sequences can optionally be specified as all (or each, independently) being those of the Kabat numbering system, those of the Chotia numbering, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody or antibody fragment, wherein the antibody or antibody fragment comprises: a HCDR1 region of 12D12 comprising an amino acid sequence SYWVH (SEQ ID NO: 30), or a sequence of at least 3 or 4 contiguous amino acids thereof, optionally wherein one or more of these amino acids may be substituted by a different amino acid; a HCDR2 region of 12D12 comprising an amino acid sequence VIDPSDSYTSYNQNFKG (SEQ ID NO: 31), or a sequence of at least 4, 5, 6, 7, 8, 9 or 10 contiguous amino acids thereof, optionally wherein one or more of these amino acids may be substituted by a different amino acid; a HCDR3 region of 12D12 comprising an amino acid sequence GERYDGDYFAMDY (SEQ ID NO: 32), or a sequence of at least 4, 5, 6, 7, 8, 9 or 10 contiguous amino acids thereof, optionally wherein one or more of these amino acids may be substituted by a different amino acid; a LCDR1 region of 12D12 comprising an amino acid sequence RASENIYSNLA (SEQ ID NO: 33), or a sequence of at least 4, 5, 6, 7, 8, 9 or 10 contiguous amino acids thereof, optionally wherein one or more of these amino acids may be substituted by a different amino acid; a LCDR2 region of 12D12 comprising an amino acid sequence AATNLAD (SEQ ID NO: 34), or a sequence of at least 4, 5 or 6 contiguous amino acids thereof, optionally wherein one or more of these amino acids may be substituted by a different amino acid; a LCDR3 region of 12D12 comprising an amino acid sequence QHFWNTPRT (SEQ ID NO: 35), or a sequence of at least 4, 5, 6 or 7 contiguous amino acids thereof, optionally wherein one or more of these amino acids may be deleted or substituted by a different amino acid.

The respective VH and VL and antibodies 3H5, 27C10 and 27H5 are shown in SEQ ID NOS: 36-37, 38-39 and 40-41, respectively. The HCDR1, 2, 3 and LCDR1, 2, 3 sequences of the antibodies can optionally be specified as all (or each, independently) being those of the Kabat numbering system, those of the Chotia numbering, those of the IMGT numbering, or any other suitable numbering system.

In another aspect of any of the embodiments herein, a heavy chain CDR (e.g., CDR1, 2 and/or 3) may be characterized as being encoded by, or derived from, a murine IGHV1 (e.g., a IGHV1-66 or IGHV1-66*01, or a IGHV1-84 or IGHV1-84*01) gene, or by a rat, non-human primate or human gene corresponding thereto, or at least 80%, 90%, 95%, 98% or 99% identical thereto. In another aspect of any of the embodiments herein, a light chain CDR (e.g., CDR1, 2 and/or 3) may be characterized as being encoded by, or derived from, a murine IGKV3 gene (e.g. IGKV3-4 or IGKV3-4*01, or a IGKV3-5 or IGKV3-5*01 gene), or by a rat, non-human primate or human gene corresponding thereto, or at least 80%, 90%, 95%, 98% or 99% identical thereto.

In another aspect of any of the embodiments herein, a heavy chain CDR (e.g., CDR1, 2 and/or 3) may be characterized as being encoded by, or derived from, a murine IGHV2 (e.g., a IGHV1-3 or IGHV1-3*01 gene, or by a rat, non-human primate or human gene corresponding thereto, or at least 80%, 90%, 95%, 98% or 99% identical thereto. In another aspect of any of the embodiments herein, a light chain CDR (e.g., CDR1, 2 and/or 3) may be characterized as being encoded by, or derived from, a murine IGKV10 gene (e.g. IGKV10-96 or IGK10-96*02), or by a rat, non-human primate or human gene corresponding thereto, or at least 80%, 90%, 95%, 98% or 99% identical thereto.

In another aspect of any of the embodiments herein, a heavy chain CDR (e.g., CDR1, 2 and/or 3) may be characterized as being encoded by a murine IGHV1 or IGHV1-84 gene (e.g., IGHV1-84*01) gene. In another aspect of any of the embodiments herein, a light chain CDR (e.g., CDR1, 2 and/or 3) may be characterized as being encoded by a murine IGKV3 or IGKV3-5 gene (e.g., IGKV3-5*01).

In another aspect of any of the embodiments herein, any of the CDRs 1, 2 and 3 of the heavy and light chains of 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO.

Optionally, in any embodiment, an 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11 B, 4E3A, 4E3B, 4H3, 5D9, 6C6 or 48F12 antibody can be specified as having a heavy chain comprising part or all of an antigen binding region of the respective antibody (e.g. heavy chain CDR1, 2 and 3), fused to an immunoglobulin heavy chain constant region of the human IgG type, optionally a human IgG1, IgG2, IgG3 or IgG4 isotype, optionally further comprising an amino acid substitution to reduce effector function (binding to human Fcγ receptors). Optionally, in any embodiment, an 12D12, 26D8, 18E1, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 antibody can be specified as having a light chain comprising part or all of an antigen binding region of the respective antibody (e.g. light chain CDR1, 2 and 3), fused to an immunoglobulin light chain constant region of the human kappa type.

The amino acid sequence of the respective heavy and light chain variable regions of antibodies 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 and 48F12 are listed in Table A. In a specific embodiment, provided is an antibody that binds essentially the same epitope or determinant as monoclonal antibodies 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12; optionally the antibody comprises the hypervariable region of antibody 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. In any of the embodiments herein, antibody 26D8 can be characterized by the amino acid sequences and/or nucleic acid sequences encoding it. In one embodiment, the monoclonal antibody comprises the Fab or F(ab′)₂ portion of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. Also provided is an antibody or antibody fragment that comprises the heavy chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. According to one embodiment, the antibody or antibody fragment comprises the three CDRs of the heavy chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. Also provided is an antibody or antibody fragment that further comprises the variable light chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 or one, two or three of the CDRs of the light chain variable region of 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In another aspect, provided is an antibody or antibody fragment (or respective VH or VL domain thereof) comprising:

a HCDR1 region (Kabat positions 31-35) of 2H2B comprising an amino acid sequence NYYMQ (SEQ ID NO: 139), or a sequence of at least 3, 4 or 5 contiguous amino acids thereof, optionally wherein one or more of these amino acids may be substituted by a different amino acid, optionally wherein the HCDR1 (or VH) comprises an amino acid substitution at Kabat position 32, 33, 34 and/or 35, optionally wherein the HCDR1 (or VH) comprises at least two aromatic residues (e.g. a Y, H or F) at Kabat position 32, 33, 34 and/or 35, optionally wherein the HCDR1 (or VH) comprises an aromatic residue at Kabat position 32 and/or an aromatic residue, N or Q at 35;

a HCDR2 region (Kabat positions 50-65) of 2H2B comprising an amino acid sequence WIFPGSGESSYNEKFKG (SEQ ID NO: 140) or WIFPGSGESNYNEKFKG (SEQ ID NO: 161), or a sequence of at least 4, 5, 6, 7, 8, 9 or 10 contiguous amino acids thereof, optionally wherein one or more of these amino acids may be substituted by a different amino acid, optionally wherein one or more of these amino acids may be substituted by a different amino acid, optionally wherein the HCDR2 (or VH) comprises an amino acid substitution at Kabat position 52A, 54, 55, 56, 57, 58, 60 and/or 65, optionally wherein the residue at 52A is P or L, optionally wherein the residue at 54 is G, S, N or T, optionally wherein the residue at 55 is G, N or Y, optionally wherein the residue at 56 is E or D, optionally wherein the residue at 57 is S or T, optionally wherein the residue at 58 is S, K or N, optionally wherein the residue at 60 is N or S, optionally wherein the residue at 65 is G or V;

a HCDR3 region (Kabat positions 95-102) of 2H2B comprising an amino acid sequence TWNYDARWGY (SEQ ID NO: 141), or a sequence of at least 4, 5, 6, 7, 8, 9 or 10 contiguous amino acids thereof, optionally wherein one or more of these amino acids may be substituted by a different amino acid, optionally wherein the HCDR3 (or VH) comprises an amino acid substitution at Kabat position 95, optionally wherein the residue at 95 is T or S, optionally wherein the HCDR3 (or VH) comprises an amino acid substitution at Kabat position 101, optionally wherein the residue at 101 is G or V;

a Kabat LCDR1 region (Kabat positions 34-34) of 2H2B comprising an amino acid sequence IPSESIDSYGISFMH (SEQ ID NO: 142), or a sequence of at least 4, 5, 6, 7, 8, 9 or 10 contiguous amino acids thereof, optionally wherein one or more of these amino acids may be substituted by a different amino acid, optionally wherein the LCDR1 (or VL) comprises an amino acid substitution at Kabat position 24, 25, 26, 27, 27A, 28, 33 and/or 34, optionally wherein the residue at 24 is I or R, optionally wherein the residue at 25 is A, P or V, optionally wherein the residue at 26 is S or N, optionally wherein the residue at 27 is E or D, optionally wherein the residue at 27A is S, G, T, I or N, optionally wherein the residue at 28 is Y or F, optionally wherein the residue at 33 is M, I or L, optionally wherein the residue at 34 is H or S, optionally wherein the LCDR1 (or VL) comprises an amino acid deletion at Kabat position 29, 30 31 and/or 32;

a Kabat LCDR2 region (Kabat positions 50-56) of 2H2B comprising an amino acid sequence RASNLES (SEQ ID NO: 143), or a sequence of at least 4, 5, or 6 contiguous amino acids thereof, optionally wherein one or more of these amino acids may be substituted by a different amino acid, optionally wherein one or more of these amino acids may be substituted by a different amino acid, optionally wherein the LCDR2 (or VL) comprises an amino acid substitution at Kabat position 50, 53 and/or 55, optionally wherein the residue at 50 is R or G, optionally wherein the residue at 53 is N, T or I, optionally wherein the residue at 54 is D, E or V;

a Kabat LCDR3 region (Kabat positions 89-97) of 2H2B comprising an amino acid sequence QQSNEDPFT (SEQ ID NO: 144), or a sequence of at least 4, 5, 6, 7, or 8 contiguous amino acids thereof, optionally wherein one or more of these amino acids may be deleted or substituted by a different amino acid, optionally wherein the LCDR3 (or VL) comprises an amino acid substitution at Kabat position 91, 94 and/or 96, optionally wherein the residue at 91 is S or T, optionally wherein the residue at 94 is D or A, optionally wherein the residue at 96 is F or W.

In another aspect, provided is an antibody or antibody fragment comprising: a HCDR1 region of 2A8A comprising an amino acid sequence NFYIH (SEQ ID NO: 145); a HCDR2 region of 2A8A comprising an amino acid sequence WIFPGSGETKFNEKFKV (SEQ ID NO: 146); a HCDR3 region of 2A8A comprising an amino acid sequence SWNYDARWGY (SEQ ID NO: 147); a LCDR1 region of 2A8A comprising an amino acid sequence RASESIDSYGISFLH (SEQ ID NO: 148); a LCDR2 region of 2A8A comprising an amino acid sequence RASNLES (SEQ ID NO: 149); a LCDR3 region of 2A8A comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2C4 comprising an amino acid sequence NYYVQ (SEQ ID NO: 151); a HCDR2 region of 2C4 comprising an amino acid sequence WIFPGSGETNYNEKFKA (SEQ ID NO: 152); a HCDR3 region of 2C4 comprising an amino acid sequence TWNYDARWGY (SEQ ID NO: 141); a LCDR1 region of 2C4 comprising an amino acid sequence RPSENIDSYGISFMH (SEQ ID NO: 181); a LCDR2 region of 2C4 comprising an amino acid sequence RASNLES (SEQ ID NO: 149); a LCDR3 region of 2C4 comprising an amino acid sequence QQTNEDPFT (SEQ ID NO: 153), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E2B comprising an amino acid sequence NYYMQ (SEQ ID NO: 154); a HCDR2 region of 2E2B comprising an amino acid sequence WIFPGGGESNYNEKFKG (SEQ ID NO: 155); a HCDR3 region of 2E2B comprising an amino acid sequence TWNYDARWGY (SEQ ID NO: 141); a LCDR1 region of 2E2B comprising an amino acid sequence IPSESIDSYGISFMH (SEQ ID NO: 156); a LCDR2 region of 2E2B comprising an amino acid sequence RASNLES (SEQ ID NO: 149); a LCDR3 region of 2E2B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2C8 comprising an amino acid sequence NYYIQ (SEQ ID NO: 157); a HCDR2 region of 2C8 comprising an amino acid sequence WIFPGNGETNYNEKFKG (SEQ ID NO: 158); a HCDR3 region of 2C8 comprising an amino acid sequence TWNYDARWGY (SEQ ID NO: 141); a LCDR1 region of 2C8 comprising an amino acid sequence RANESIDSYGISFMH (SEQ ID NO: 159); a LCDR2 region of 2C8 comprising an amino acid sequence RASNLDS (SEQ ID NO: 160); a LCDR3 region of 2C8 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E2C comprising an amino acid sequence NYYMQ (SEQ ID NO: 154); a HCDR2 region of 2E2C comprising an amino acid sequence WIFPGSGESNYNEKFKG (SEQ ID NO: 161); a HCDR3 region of 2E2C comprising an amino acid sequence TWNYDARWGY (SEQ ID NO: 141); a LCDR1 region of 2E2C comprising an amino acid sequence IPSESIDSYGISFMH (SEQ ID NO: 162); a LCDR2 region of 2E2C comprising an amino acid sequence RASNLES (SEQ ID NO: 149); a LCDR3 region of 2E2C comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2A9 comprising an amino acid sequence NYYIH (SEQ ID NO: 163); a HCDR2 region of 2A9 comprising an amino acid sequence WIFPGSGETNYNEKFKV (SEQ ID NO: 164); a HCDR3 region of 2A9 comprising an amino acid sequence TWNYDARWGY (SEQ ID NO: 141); a LCDR1 region of 2A9 comprising an amino acid sequence RASESIDSYGISFMH (SEQ ID NO: 165); a LCDR2 region of 2A9 comprising an amino acid sequence RASNLES (SEQ ID NO: 149); a LCDR3 region of 2A9 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E11 comprising an amino acid sequence NYYIH (SEQ ID NO: 163); a HCDR2 region of 2E11 comprising an amino acid sequence WIFPGSGDTNYNEKFKG (SEQ ID NO: 166); a HCDR3 region of 2E11 comprising an amino acid sequence TWNYDARWGY (SEQ ID NO: 141); a LCDR1 region of 2E11 comprising an amino acid sequence RVSESIDSYGISFMH (SEQ ID NO: 167); a LCDR2 region of 2E11 comprising an amino acid sequence RASTLES (SEQ ID NO: 168); a LCDR3 region of 2E11 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2E8 comprising an amino acid sequence NFYIH (SEQ ID NO: 145); a HCDR2 region of 2E8 comprising an amino acid sequence WIFPGNGETNYSEKFKG (SEQ ID NO: 169); a HCDR3 region of 2E8 comprising an amino acid sequence TWNYDARWVY (SEQ ID NO: 170); a LCDR1 region of 2E8 comprising an amino acid sequence RASDGIDSYGISFMH (SEQ ID NO: 171); a LCDR2 region of 2E8 comprising an amino acid sequence RASILES (SEQ ID NO: 172); a LCDR3 region of 2E8 comprising an amino acid sequence QQTNEDPFT (SEQ ID NO: 153), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2H12 comprising an amino acid sequence NFYIH (SEQ ID NO: 145); a HCDR2 region of 2H12 comprising an amino acid sequence WIFPGNGETNYSEKFKG (SEQ ID NO: 173); a HCDR3 region of 2H12 comprising an amino acid sequence TWNYDARWGY (SEQ ID NO: 141); a LCDR1 region of 2H12 comprising an amino acid sequence RASDGIDSYGISFMH (SEQ ID NO: 174); a LCDR2 region of 2H12 comprising an amino acid sequence RASTLES (SEQ ID NO: 168); a LCDR3 region of 2H12 comprising an amino acid sequence QQTNEAPFT (SEQ ID NO: 175), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 1E4B comprising an amino acid sequence NYYIN (SEQ ID NO: 176); a HCDR2 region of 1E4B comprising an amino acid sequence WIFPGNGDTNYNEKFKG (SEQ ID NO: 177); a HCDR3 region of 1E4B comprising an amino acid sequence TWNYDARWGY (SEQ ID NO: 141); a LCDR1 region of 1E4B comprising an amino acid sequence RASESIDSYMS (SEQ ID NO: 178); a LCDR2 region of 1E4B comprising an amino acid sequence GASNLES (SEQ ID NO: 179); a LCDR3 region of 1E4B comprising an amino acid sequence QQSNEDPWT (SEQ ID NO: 180), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E5 comprising an amino acid sequence NFYIH (SEQ ID NO: 145); a HCDR2 region of 3E5 comprising an amino acid sequence WIFPGTGETNFNEKFKV (SEQ ID NO: 182); a HCDR3 region of 3E5 comprising an amino acid sequence SWNYDARWGY (SEQ ID NO: 183); a LCDR1 region of 3E5 comprising an amino acid sequence RASESIDSFGISFMH (SEQ ID NO: 184); a LCDR2 region of 3E5 comprising an amino acid sequence RASNLES (SEQ ID NO: 149); a LCDR3 region of 3E5 comprising an amino acid sequence QQSNEAPFT (SEQ ID NO: 185), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E7A comprising an amino acid sequence NYYIH (SEQ ID NO: 163); a HCDR2 region of 3E7A comprising an amino acid sequence WIFPGSGETNFNEKFKG (SEQ ID NO: 186); a HCDR3 region of 3E7A comprising an amino acid sequence TWNYDARWGY (SEQ ID NO: 141); a LCDR1 region of 3E7A comprising an amino acid sequence RASESIDSYGISFMH (SEQ ID NO: 187); a LCDR2 region of 3E7A comprising an amino acid sequence RASNLES (SEQ ID NO: 149); a LCDR3 region of 3E7A comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E7A or 3E7B comprising an amino acid sequence NYYIH (SEQ ID NO: 163); a HCDR2 region of 3E7A or 3E7B comprising an amino acid sequence WIFPGSGETNFNEKFKG (SEQ ID NO: 188); a HCDR3 region of 3E7A or 3E7B comprising an amino acid sequence TWNYDARWGY (SEQ ID NO: 141); a LCDR1 region of 3E7A or 3E7B comprising an amino acid sequence RASESIDSYGISFMH (SEQ ID NO: 189); a LCDR2 region of 3E7A or 3E7B comprising an amino acid sequence RASNLES (SEQ ID NO: 149) or RASNLVS (SEQ ID NO: 190); a LCDR3 region of 3E7A or 3E7B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3E9B comprising an amino acid sequence NYYIH (SEQ ID NO: 163); a HCDR2 region of 3E9B comprising an amino acid sequence WIFPGSGETNYNEKFKG (SEQ ID NO: 191); a HCDR3 region of 3E9B comprising an amino acid sequence TWNYDARWGY (SEQ ID NO: 141); a LCDR1 region of 3E9B comprising an amino acid sequence RASETIDSYGISFMH (SEQ ID NO: 192); a LCDR2 region of 3E9B comprising an amino acid sequence RASNLES (SEQ ID NO: 149); a LCDR3 region of 3E9B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 3F5 comprising an amino acid sequence NYYIQ (SEQ ID NO: 157); a HCDR2 region of 3F5 comprising an amino acid sequence WIFPGNNETNYNEKFKG (SEQ ID NO: 193); a HCDR3 region of 3F5 comprising an amino acid sequence SWNYDARWGY (SEQ ID NO: 147); a LCDR1 region of 3F5 comprising an amino acid sequence RASEIIDSYGISFMH (SEQ ID NO: 194); a LCDR2 region of 3F5 comprising an amino acid sequence RASNLES (SEQ ID NO: 149); a LCDR3 region of 3F5 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 4C11B comprising an amino acid sequence NYYIH (SEQ ID NO: 163); a HCDR2 region of 4C11B comprising an amino acid sequence WIFPGSGETNYSEKFKG (SEQ ID NO: 195); a HCDR3 region of 4C11B comprising an amino acid sequence SWNYDARWGY (SEQ ID NO: 147); a LCDR1 region of 4C11B comprising an amino acid sequence RASESIDSYGISFMH (SEQ ID NO: 196); a LCDR2 region of 4C11B comprising an amino acid sequence RASNLES (SEQ ID NO: 149); a LCDR3 region of 4C11B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 4E3A or 4E3B comprising an amino acid sequence NYYIQ (SEQ ID NO: 157); a HCDR2 region of 4E3A or 4E3B comprising an amino acid sequence WIFPGSGETNYNENFKA (SEQ ID NO: 197) or WIFPGSGETNYNENFRA (SEQ ID NO: 198); a HCDR3 region of 4E3A or 4E3B comprising an amino acid sequence TWNYDARWGY (SEQ ID NO: 141); a LCDR1 region of 4E3A or 4E3B comprising an amino acid sequence RPSENIDSYGISFMH (SEQ ID NO: 199); a LCDR2 region of 4E3A or 4E3B comprising an amino acid sequence RASNLES (SEQ ID NO: 149); a LCDR3 region of 4E3A or 4E3B comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 4H3 comprising an amino acid sequence NYYIH (SEQ ID NO: 163); a HCDR2 region of 4H3 comprising an amino acid sequence WIFPGSGDTNYNEKFKG (SEQ ID NO: 200); a HCDR3 region of 4H3 comprising an amino acid sequence TWNYDARWGY (SEQ ID NO: 141); a LCDR1 region of 4H3 comprising an amino acid sequence RVSESIDSYGISFMH (SEQ ID NO: 201); a LCDR2 region of 4H3 comprising an amino acid sequence RASTLES (SEQ ID NO: 168); a LCDR3 region of 4H3 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 5D9 comprising an amino acid sequence NYYIH (SEQ ID NO: 163); a HCDR2 region of 5D9 comprising an amino acid sequence WIFLGSGETNYNEKFKG (SEQ ID NO: 202); a HCDR3 region of 5D9 comprising an amino acid sequence SWNYDARWGY (SEQ ID NO: 147); a LCDR1 region of 5D9 comprising an amino acid sequence RASESIDSYGISFIH (SEQ ID NO: 203); a LCDR2 region of 5D9 comprising an amino acid sequence RASNLES (SEQ ID NO: 149); a LCDR3 region of 5D9 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 6C6 comprising an amino acid sequence NFYIH (SEQ ID NO: 145); a HCDR2 region of 6C6 comprising an amino acid sequence WIFPGSGETNYNERFKG (SEQ ID NO: 204); a HCDR3 region of 6C6 comprising an amino acid sequence SWNYDARWGY (SEQ ID NO: 147); a LCDR1 region of 6C6 comprising an amino acid sequence RASESIDSYGISFMH (SEQ ID NO: 205); a LCDR2 region of 6C6 comprising an amino acid sequence RASNLES (SEQ ID NO: 149); a LCDR3 region of 6C6 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 2D8 comprising an amino acid sequence NFYIH (SEQ ID NO: 145); a HCDR2 region of 2D8 comprising an amino acid sequence WIFPGSGETNFNEKFKV (SEQ ID NO: 206); a HCDR3 region of 2D8 comprising an amino acid sequence SWNYDARWGY (SEQ ID NO: 147); a LCDR1 region of 2D8 comprising an amino acid sequence RASESVDSYGISFMH (SEQ ID NO: 207); a LCDR2 region of 2D8 comprising an amino acid sequence RASILES (SEQ ID NO: 172); a LCDR3 region of 2D8 comprising an amino acid sequence QQSNEDPFT (SEQ ID NO: 150), Optionally, any CDR sequence can be characterized as a sequence of at least 4, 5, 6 or 7 contiguous amino acids of the listed sequence, optionally wherein one or more of these amino acids may be deleted or substituted by a different amino acid.

In another aspect, provided is an antibody or antibody fragment comprising: a HCDR1 region of 48F12 comprising an amino acid sequence SYGVS (SEQ ID NO: 208); a HCDR2 region of 48F12 comprising an amino acid sequence IIWGDGSTNYHSALVS (SEQ ID NO: 209); a HCDR3 region of 48F12 comprising an amino acid sequence PNWDYYAMDY (SEQ ID NO: 210); a LCDR1 region of 48F12 comprising an amino acid sequence RASQDISNYLN (SEQ ID NO: 211); a LCDR2 region of 48F12 comprising an amino acid sequence YTSRLHS (SEQ ID NO: 212); a LCDR3 region of 48F12 comprising an amino acid sequence QQGITLPLT (SEQ ID NO: 213), Optionally, any CDR sequence can be characterized as a sequence of at least 4, 5, 6 or 7 contiguous amino acids of the listed sequence, optionally wherein one or more of these amino acids may be deleted or substituted by a different amino acid.

In any of the antibodies, e.g., 12D12, 26D8, 18E1, 27C10, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12, the specified variable region and CDR sequences may comprise sequence modifications, e.g. a substitution (1, 2, 3, 4, 5, 6, 7, 8 or more sequence modifications). In one embodiment, any one or more (or all of) CDRs 1, 2 and/or 3 of the heavy and light chains comprises one, two, three or more amino acid substitutions, optionally where the residue substituted is a residue present in a sequence of human origin. In one embodiment the substitution is a conservative modification. A conservative sequence modification refers to an amino acid modification that does not significantly affect or alter the binding characteristics of the antibody containing the amino acid sequence. Such conservative modifications include amino acid substitutions, additions and deletions. Modifications can be introduced into an antibody by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for retained function (i.e., the properties set forth herein) using the assays described herein.

Optionally, in any embodiment, a VH may comprise an amino acid substitution at Kabat position 32, 33, 34 and/or 35. A VH may comprise an amino acid substitution at Kabat position 52A, 54, 55, 56, 57, 58, 60 and/or 65. In any embodiment, a VH may comprise an amino acid substitution at Kabat position 95 and/or 101. In any embodiment, a VL may comprise an amino acid substitution at Kabat position 24, 25, 26, 27, 27A, 28, 33 and/or 34, and/or an amino acid deletion at Kabat position 29, 30 31 and/or 32. In any embodiment, a VL may comprise an amino acid substitution at Kabat position 50, 53 and/or 55. In any embodiment, a VL may comprise an amino acid substitution at Kabat position 91, 94 and/or 96.

Optionally, in any embodiment herein, an anti-ILT2 antibody can be characterized as being a function-conservative variant of any of the antibodies, heavy and/or light chains, CDRs or variable regions thereof described herein. “Function-conservative variants” are those in which a given amino acid residue in a protein or antibody has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein (e.g. heavy or light chains, or CDRs or variable regions thereof) to which it is compared. In one embodiment, the antibody comprises a heavy chain variable region that is a function-conservative variant of the heavy chain variable region of antibody 2H2B, 48F12, 3F5, 12D12, 26D8 or 18E1, and a light chain variable region that is a function-conservative variant of the light chain variable region of the respective 2H2B, 48F12, 3F5, 12D12, 26D8 or 18E1 antibody. In one embodiment, the antibody comprises a heavy chain that is a function-conservative variant of the heavy chain variable region of antibody 2H2B, 48F12, 3F5, 12D12, 26D8 or 18E1 fused to a human heavy chain constant region disclosed herein, optionally a human IgG4 constant region, optionally a modified IgG (e.g. IgG1) constant region, e.g. a constant region of any of SEQ ID NOS: 42-45, and a light chain that is a function-conservative variant of the light chain variable region of the respective 2H2B, 48F12, 3F5, 12D12, 26D8 or 18E1 antibody fused to a human Ckappa light chain constant region.

TABLE A

| Antibody | SEQ ID | |
|---|---|---|
| domain | NO: | Amino Acid Sequence |
| 26D8 VH | 12 | QVQLQQSGAELVKPGASVKLSCKASGYTFTEHTIHWIKQRSGQGLEWIGW |
| | | FYPGSGSMKYNEKFKDKATLTADKSSSTVYMELTRLTSEDSAVYFCARHT |
| | | NWDFDYWGQGTTLTVSS |
| 26D8 VL | 13 | DIVLTQSPASLAVSLGQRATISCKASQSVDYGGDSYMNWYQQKPGQPPKL |
| | | LIYAASNLESGIPARFSGSGSGTDLTLNIHPVEEDDAAMYYCQQSNEEPW |
| | | TFGGGTKLEIK |
| 18E1 VH | 20 | QVQLQQSGAELVKPGASVRLSCKASGYTFTAHTIHWVKQRSGQGLEWIGW |
| | | LYPGSGSIKYNEKFKDKATLTADKSSSTVYMELSRLTSEDSAVYFCARHT |
| | | NWDFDYWGQGTTLTVSS |
| 18E1 VL | 21 | NIVLTQSPASLAVSLGQRATISCKASQSVDYGGASYMNWYQQKPGQPPKL |
| | | LIYAASNLESGIPARFSGSGSGTDLTLNIHPVEEEDAAMYYCQQSNEEPW |
| | | TFGGGTKLEIK |
| 12D12VH | 28 | QVQLQQPGAELVKPGASVRMSCKASGYTFTSYWVHWVKQRPGQGLEWIGV |
| | | IDPSDSYTSYNQNFKGKATLTVDTSSKTAYIHLSSLTSEDSAVYFCARGE |
| | | RYDGDYFAMDYWGQGTSVTVSS |
| 12D12 VL | 29 | DIVMTQSPASLSVSVGETVTITCRASENIYSNLAWYQQKQGKSPQLLVYA |
| | | ATNLADGVPSRFSGSRSGTQYSLKINSLQSEDFGTYYCQHFWNTPRTFGG |
| | | GTKLEIK |
| 3H5 VH | 36 | QVQLKESGPGLVAPSQSLSITCTVSGFSLTSYGVSWVRQPPGKGLEWLGV |
| | | IWGDGSTNYHSALISRLSISKDNSKSQVFLKLNSLQTDDTATYYCAKPRW |
| | | DDYAMDYWGQGTSVTVSS |
| 3H5 VL | 37 | DIQMTQTTSSLSASLGDRVTISCRASQDISNYLNWYQQKPDGTVKLLIYY |
| | | TSRLHSGVPSRFSGSGSGTDYSLTISNLEQEDIATYFCQQGNTLWTFGGG |
| | | TKLEIK |
| 27C10 VH | 38 | EVQLQESGPGLVKPSQSLSLTCSVTGYSITSGYYWNWIRQFPENKLEWMG |
| | | YIRYDGSNNYNPSLNNRISITRDASKNQFFLKLNSVTTEDTATYYCARGW |
| | | LLWFYAVDYWGQGTSVTVSS |
| 27C10 VL | 39 | DVVMTQTPLSLPVSLGDQASISCRSSQSIVHTNGNTYLEWYLQKSGQSPK |
| | | LLIYKVSNRLSGVPDRFSGSGSGTDFTLKISRVEAEDLGIYYCFQGSHVP |
| | | WTFGGGTKLEIK |
| 27H5 VH | 40 | QVQLKESGPGLVAPSQSLSITCTVSGFSLTSYGVSWVRQPPGKGLEWLGV |
| | | IWGDGNTNYHSALISRLSISKDNSKSQVFLKLNSLQTDDTATYYCARTNW |
| | | DGWFAYWGQGTLVTVSA |
| 27H5 VL | 41 | DIVMTQSHKFMSTSVGDRVSITCKASQDVGTAVAWYQQKPGQSPKLLIYW |
| | | ASTRHTGVPDRFTGSGSGTDFTLTISNVQSEDLADYFCQQYRSYPLGTFG |
| | | GGTKLEIK |
| 2A8A VH | 81 | QVQLQQSGPELVKPGASVKISCKASGYSFTNFYIHWVRQRPGQGLDWIGW |
| | | IFPGSGETKFNEKFKVKATLTADTSSSTAYMQLNSLTSEDSAVYFCARSW |
| | | NYDARWGYWGQGTSVTVSS |
| 2A8A VL | 82 | QIVLTQSPASLAVSLGQRATISCRASESIDSYGISFLHWYQQKPGQPPKL |
| | | LIYRASNLESGIPARFSGSGSRPDFTLTINPVEADDVATYYCQQSNEDPF |
| | | TFGSGTKLEIK |
| 2C4 VH | 83 | DVQLVESGPELVKPGASVKISCKASGYSFTNYYMQWVKQRPGQGLEWIGW |
| | | IFPGGGESNYNEKFKGKATLSADTSSTTAYMQLSSLTSEDSAVYFCARTW |
| | | NYDARWGYWGQGTTVTVSS |
| 2C4 VL | 84 | DIQMTQSPASLTVSLGQRATISCRPSENIDSYGISFMHWYQQKPGQPPKL |
| | | LIYRASNLESGIPVRFSGSGSRTDFTLTINPVEADDVATYYCQQTNEDPF |
| | | TFGSGTKLEIK |
| 2E2B VH | 85 | EVQLKQSGPELVKPGASVKISCKASGYSFTNYYIQWVKQRPGQGLEWIGW |
| | | IFPGNGETNYNEKFKGKATLTADTSSSTAYMQLSSLTSEDSAVYFCARTW |
| | | NYDARWGYWGQGTSVTVSS |
| 2E2B VL | 86 | DIVLTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 2C8 VH | 87 | QVQLQQSGPELVKPGASVKISCKASGYSFTNYYMQWVKQRPGQGLEWIGW |
| | | IFPGSGESNYNEKFKGKATLSADTSSTTAYMQLSSLTSEDSAVYFCARTW |
| | | NYDARWGYWGQGTSVTVSS |
| 2C8 VL | 88 | DILLTQSPASLTVSLGQRATISCRANESIDSYGISFMHWYQQKPGQPPKL |
| | | LIYRASNLDSGIPARFSGSGSRTDFTLTINPVEADDVATYYCQQSNEDPF |
| | | TFGSGTKLEIK |
| 2E2C VH | 89 | EFQLQQSGPELVKPGASVKISCKASGYSFTNYYMQWVKQRPGQGLEWIGW |
| | | IFPGSGESNYNEKFKGKATLSADTSSTTAYMQLSSLTSEDSAVYFCARTW |
| | | NYDARWGYWGQGTTLTVSS |
| 2E2C VL | 90 | DIVMTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 2H2A VH | 91 | EVKLEESGPELVKPGASVKLSCKASGYTFTNYYMQWVKQRPGQGLEWIGW |
| | | IFPGSGESSYNEKFKGKATLSADTSSTTAYMQLSSLTSEDSAVYFCARTW |
| | | NYDARWGYWGQGTTLTVSS |
| 2H2A VL | 92 | DILMTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLELK |
| 2H2B VH | 93 | EVKLQQSGPELVKPGASVKISCKASGYSFTNYYIHWVKQRPGQGLEWIGW |
| | | IFPGSGETNYNEKFKVKATLSADTSSTTAYMQLSSLTSEDSAVYFCARTW |
| | | NYDARWGYWGQGTTLTVSS |
| 2H2B VL | 94 | DILMTQSPASLAVSLGQRATISCIPSESIDSYGISFMHWYQQKPGQPPKL |
| | | LIYRASNLESGIPARFSGSGSRTDFTLTINPVEADDVATYYCQQSNEDPF |
| | | TFGSGTKLELK |
| 2A9 VH | 95 | QVQLKESGPELVKPGASVKISCKTSGYSFTNYYIHWVKQRPGQGLEWIGW |
| | | IFPGSGDTNYNEKFKGKATLTADTSSNTASMHLSSLTSEDSAVYFCARTW |
| | | NYDARWGYWGQGTTLTVSS |
| 2A9 VL | 96 | DVVVTQTPASLAVSLGQRATISCRASESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 2E11 VH | 97 | EVQLQQSGPDLVKPGASVKMSCKASGYSFTNFYIHWVKQRPGQGLEWIGW |
| | | IFPGNGETNYSEKFKGKATLTADTSSSTAYMQFNSLTYEDSAVYFCARTW |
| | | NYDARWVYWGQGTTVTVSS |
| 2E11 VL | 98 | DIVMTQSPASLAVSLGQRATISCRVSESIDSYGISFMHWYQQKSGQPPKV |
| | | LIYRASTLESGIPARFSGSGSRTDFTLTINPVEADDVATYYCQQSNEDPF |
| | | TFGSGTKLEIK |
| 2E8 VH | 99 | EVKLQQSGPDLVKPGASVKISCKASGYSFTNFYIHWVKQRPGQGLEWIGW |
| | | IFPGNGETNYSEKFKGKATLTADTSSSTAYMQFNSLTYEDSAVYFCARTW |
| | | NYDARWGYWGQGTTLTVSS |
| 2E8 VL | 100 | EIVLTQSPASLAVSLGQRATISCRASDGIDSYGISFMHWYQQKPGQPPTV |
| | | LIYRASILESGIPARFSGSGSRTDFTLTINPVEADDVATYYCQQTNEDPF |
| | | TFGSGTKLEIK |
| 2H12 VH | 101 | DVQLVESGPELVKPGASVKISCKASGYSFTNYYMQWVKQRPGQGLEWIGW |
| | | IFPGGGESNYNEKFKGKATLSADTSSTTAYMQLSSLTSEDSAVYFCARTW |
| | | NYDARWGYWGQGTTVTVSS |
| 2H12 VL | 102 | DILLTQSPASLAVSLGQRATISCRASDGIDSYGISFMHWYQQKPGQPPTL |
| | | LIYRASTLESGIPARFSGSGSRTDFTLTINPVEADDVATYYCQQTNEAPF |
| | | TFGSGTKLELK |
| 1E4B VH | 103 | DVQLQESGPELVKPGASVKISCKSSGYSFTNFYIHWVKQRPGQGLDWIGW |
| | | IFPGTGETNENEKEKVKAALTADTSSSTVYMQLSTLTSEDSAVYFCARSW |
| | | NYDARWGYWGQGTSITVSS |
| 1E4B VL | 104 | DVVMTQTPAFLAVSLGQRATISCRASESIDSYMSWYQQKPGQPPKVLIYG |
| | | ASNLESGIPARFSGSGSGTDFTLNIHPVEEEDAATYYCQQSNEDPWTEGG |
| | | GTKLEIK |
| 3E5 VH | 105 | EVQLQESGPELVKPGASVKISCKASGYSFRNYYIQWVKQRPGQGLEWIGW |
| | | IFPGNYETNYNEKFKGKATLSADTSSTTAYMQLSSLTSEDSAVYFCARSW |
| | | NYDARWGYWGQGTSVTVSS |
| 3E5 VL | 106 | ENVLTQSPASLAVSLGQRATISCRASESIDSEGISFMHWYQQKPGQPPKL |
| | | LIYRASNLESGIPARFSGSGSGPDFSLTIDPVEADDVATYYCQQSNEAPF |
| | | TFGSGTKLEIK |
| 1A10D | 107 | QVQLKQSGPELVKPGASVKISCKASGYSFTNYYIHWVKQRPGQGLEWIGW |
| VH | | IFPGSGETNENEKFKGKATLTADTSSSTAYMQESSLTSEDSAVYFCARTW |
| | | NYDARWGYWGQGTTVTVSS |
| 1A10D VL | 108 | EIVLTQSPASLAVSLGQRATISCRASESIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 3E7A VH | 109 | QVQLKQSGPELVKPGASVKISCKASGYSFTNYYIHWVKQRPGQGLEWIGW |
| | | IFPGSGETNENEKFKGKATLTADTSSSTAYMQESSLTSEDSAVYFCARTW |
| | | NYDARWGYWGQGTTVTVSS |
| 3E7A VL | 110 | DILMTQSPASLAVSLGQRATISCRASEGIDSYGISFMHWYQQKPGQPPTL |
| | | LIYRASNLESGIPARFSGSGSRTDFTLTINPVEADDVATYYCQQTNEDPF |
| | | TFGSGTKLEIK |
| 3E7B VH | 111 | EVQLQESGPELVKPGASVKISCKTSGYSFTNYYIHWVKQRPGQGLEWIGW |
| | | IFPGSGETNYNEKFKGKATLSADTSSTTAYMQLSSLTSEDSAVYFCARTW |
| | | NYDARWGYWGQGTTVTVSS |
| 3E7B VL | 112 | EIQMTQSPASLAVSLGQRATISCRASEGIDSYGISFMHWYQQKPGQPPTL |
| | | LIYRASNLVSGIPARFSGSGSRTDFTLTINPVEADDVATYYCQQTNEDPF |
| | | TFGSGTKLEIK |
| 3E9B VH | 113 | DVQLQESGPDLVKPGASVKISCKASGYSFRNYYIQWVKQRPGQGLEWIGW |
| | | IFPGNNETNYNEKFKGKATLSADTSSTTAYMQLSSLTSEDSAVYFCARSW |
| | | NYDARWGYWGQGTTLTVSS |
| 3E9B VL | 114 | EILLTQSPASLAVSLGQRATISCRASETIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 3F5 VH | 115 | QVQLKESGPELVKPGASVKISCKASGYSFTNYYIHWVKQRPGQGLEWIGW |
| | | IFPGSGETNYSEKFKGEAILTADTSSNTAYMQLSSLTSEDSAVYFCARSW |
| | | NYDARWGYWGQGTTLTVSS |
| 3F5 VL | 116 | EIVLTQSPASLAVSLGQRATISCRASEIIDSYGISFMHWYQQKPGQPPKL |
| | | LIYRASNLESGIPARFSGSGSRTDFTLTINPVEADDVATYYCQQSNEDPF |
| | | TFGSGTKLEIK |
| 4C11B | 117 | QIQLQQSGPELVKPGASVKISCKASGYSFTNYYIQWVKQRPGQGLEWIGW |
| VH | | IFPGSGETNYNENFKAKATLSADTSSTTAYMQLSSLTSEDSAVYFCARTW |
| | | NYDARWGYWGQGTSVTVSS |
| 4C11B VL | 118 | QIVLSQSPVSLAVSPGQRATISCRASESIDSYGISFMHWYKQKPGQPPKL |
| | | LIYRASNLESGIPARFSGSGSRTDFTLTINPVEADDVATYYCQQSNEDPF |
| | | TFGSGTKLEIK |
| 4E3A VH | 119 | EVHLQQSGPELVKPGASVKISCKASGYSFTNYYIQWVKQRPGQGLEWIGW |
| | | IFPGSGETNYNENFRAKATLSADTSSTTAYMQLSSLTSEDSAVYFCARTW |
| | | NYDARWGYWGQGTTVTVSS |
| 4E3A VL | 120 | EILLTQSPPASLAVSLGQRVTISCRPSENIDSYGISFMHWYQQKPGQPPK |
| | | LLIYRASNLESGIPVRFSGSGSRTDFTLTINPVEADDVATYYCQQSNEDP |
| | | FTFGSGTKLEIK |
| 4E3B VH | 121 | QVQLKESGPELVKPGASVKISCKTSGYIFTNYYIHWVKQRPGQGLEWIGW |
| | | IFPGSGDTNYNEKFKGKATLTADTSSSTASMQLSSLTSEDSAVYFCARTW |
| | | NYDARWGYWGQGTTVTVSS |
| 4E3B VL | 122 | DILLTQSPASLAVSLGQRATISCRPSENIDSYGISFMHWCQQKPGQPPKL |
| | | LIYRASNLESGIPVRFSGSGSRTDFTLTINPVEADDVATYYCQQSNEDPF |
| | | TFGSGTKLEIK |
| 4H3 VH | 123 | QVQLKESGPELVKPGASVKISCKASGYSFTNYYIHWVKQRPGQGLEWIGW |
| | | IFLGSGETNYNEKFKGEAILTADTSSTTAYMQLSSLTSEDSAVYFCARSW |
| | | NYDARWGYWGQGTTLTVSS |
| 4H3 VL | 124 | DILLTQSPASLAVSLGQRATISCRVSESIDSYGISFMHWYQQKSGQPPKV |
| | | LIYRASTLESGIPARFSGSGSRTDFTLTINPVEADDVATYYCQQSNEDPF |
| | | TFGSGTKLEIK |
| 5D9 VH | 125 | EVQLQQSGPELVKPGASVKISCKASGYSFTNFYIHWVKQRPGQGLDWIGW |
| | | IFPGSGETNYNERFKGKATLTSDTSSSTAYMQLSSLTSEDSAVYFCARSW |
| | | NYDARWGYWGQGTTLTVSS |
| 5D9 VL | 126 | EIVLTQSPASLAVSLGQRATISCRASESIDSYGISFIHWYQQKPGQPPKL |
| | | LIYRASNLESGIPARFSGSGSRTDFTLTINPVEAEDVATYYCQQSNEDPF |
| | | TFGSGTKLEIK |
| 6C6 VH | 127 | EVQLQQSGPELVKPGASVKISCKSSGYSFTNFYIHWVKQRPGQGLDWIGW |
| | | IFPGSGETNFNEKFKVKAALTADTSSNTAYMQLSSLTSEDSAVYFCARSW |
| | | NYDARWGYWGQGTTVTVSS |
| 6C6 VL | 128 | QIVLTQTPASLAVSLGQRATISCRASESIDSYGISFMHWYQQKPGQPPKL |
| | | LIYRASNLESGIPARFSGSGSRPDFTLTINPVEADDVATYYCQQSNEDPF |
| | | TFGSGTKLEIK |
| 2D8 VH | 129 | QVQLKESGPGLVAPSQSLSITCTVSGFSLTSYGVSWVRQPPGKGLEWLGI |
| | | IWGDGSTNYHSALVSRLSISKDNSKSQVFLKLNSLQTDDTATYYCAKPNW |
| | | DYYAMDYWGQGTSVTVSS |
| 2D8 VL | 130 | DAVMTQTPASLAVSLGQRATISCRASESVDSYGISFMHWYQQKPGQPPKL |
| | | LIYRASILESGIPARFSGSGSRPDFSLTINPVEADDVATYYCQQSNEDPF |
| | | TFGSGTKLEIK |
| 48F12 VH | 131 | DVQLQESGPELVKPGASVKISCKSSGYSFTNFYIHWVKQRPGQGLDWIGW |
| | | IFPGTGETNFNEKFKVKAALTADTSSSTVYMQLSTLTSEDSAVYFCARSW |
| | | NYDARWGYWGQGTSITVSS |
| 48F12 VL | 132 | DIQMTQTTSSLSASLGDRVTISCRASQDISNYLNWYQQKVDGTVKLLISY |
| | | TSRLHSGVPSRFSGSGSGTDYSLTISNLEQEDIATYFCQQGITLPLTFGA |
| | | GTKLELK |
| 1A9 VH | 133 | EVKLQQSGPDLVKPGASVKISCKASGYSFTNFYIHWVKQRPGQGLEWIGW |
| | | IFPGNGETNYSEKFKGKATLTADTSSSTAYMQFNSLTYEDSAVYFCARTW |
| | | NYDARWGYWGQGTTLTVSS |
| 1A9 VL | 134 | DVVMTQTPASLAVSLGQRATISCRASDGIDSYGISFMRWYQQKPGQPPTL |
| | | LIYRASTLESGIPARFSGSGSRTNFTLTINPVEADDVATYYCQQTNEDPF |
| | | TFGSGTKLEIK |
| 1E4C VH | 135 | QRELQQSGPELVKPGASVNISCKASGYSFTNHYINWVKQRPGQGLEWIGW |
| | | IFPGNGDTNYNEKFKGKATLTADTSSSTAYMQLSSLTSEDSAVYFCARTW |
| | | NYDARWGYWGQGTTVTVSS |
| 1E4C VL | 136 | DVVMTQTPAFLAVSLGQRATISCRASESIDSYGISFMHWYQQKPGQPPKV |
| | | LIYRTSNLESGIPARFSGSGSRTDFTLTINPVEADDVATYYCQQSNEDPF |
| | | TFGSGTKLEIK |
| 3A7A VH | 137 | QVQLKESGPELVKPGTSVKISCKASGYNFRNYYIQWVKQRPGQGLEWIGW |
| | | IFPGNNETNYNEKFKGKATLSADTSSTTAYMQLSSLTSEDSAVYFCARSW |
| | | NYDARWGYWGQGTTVTVSS |
| 3A7A VL | 138 | DVVMTQTPASLAVSLGQRATISCRASEIIDNYGISFIHWYQQKPGQPPKL |
| | | LIYRASNLESGIPARFSGSGSRTDSTLTINPVGADDVATYYCQQSNEDPF |
| | | TFGSGTKLELK |

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific (e.g., bispecific) antibodies formed from antibody fragments. Included, inter alia, are a nanobody, domain antibody, single domain antibody or a “dAb”.

In certain embodiments, the DNA of a hybridoma producing an antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody.

Optionally an antibody is humanized. “Humanized” forms of antibodies are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies are well known in the art.

The choice of human variable domains, both light and heavy, to be used in making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for ILT receptors and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding.

Another method of making “humanized” monoclonal antibodies is to use a XenoMouse (Abgenix, Fremont, Calif.) as the mouse used for immunization. A XenoMouse is a murine host according that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

In one embodiment, the anti-ILT2 antibodies can be prepared such that they do not have substantial specific binding to human FCγ receptors, e.g., any one or more of CD16A, CD16B, CD32A, CD32B and/or CD64). Such antibodies may comprise constant regions of various heavy chains that are known to lack or have low binding to FCγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in a BIACORE assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such as cell based assays, to assess Fc receptor binding are well known in the art, and are described in, e.g., WO 03/101485.

In one embodiment, the antibody can comprise one or more specific mutations in the Fc region that result in antibodies that have minimal interaction with effector cells. Silenced effector functions can be obtained by mutation in the Fc region of the antibodies and have been described in the art: N297A mutation, the LALA mutations, (Strohl, W., 2009, Curr. Opin. Biotechnol. vol. 20(6):685-691); and D265A (Baudino et al., 2008, J. Immunol. 181: 6664-69) see also Heusser et al., WO2012/065950, the disclosures of which are incorporated herein by reference. In one embodiment, an antibody comprises one, two, three or more amino acid substitutions in the hinge region. In one embodiment, the antibody is an IgG1 or IgG2 and comprises one, two or three substitutions at residues 233-236, optionally 233-238 (EU numbering). In one embodiment, the antibody is an IgG4 and comprises one, two or three substitutions at residues 327, 330 and/or 331 (EU numbering). Examples of silenced Fc IgG1 antibodies are the LALA mutant comprising L234A and L235A mutation in the IgG1 Fc amino acid sequence. Another example of an Fc mutation is a mutation at residue D265, or at D265 and P329 for example as used in an IgG1 antibody as the DAPA (D265A, P329A) mutation (U.S. Pat. No. 6,737,056). Another modified IgG1 antibody comprises a mutation at residue N297 (e.g., N297A, N297S mutation), which results in aglycosylated/non-glycosylated antibodies. Other mutations that reduce and/or abrogate FcgammaR-interactions include: substitutions at residues L234 and G237 (L234A/G237A); substitutions at residues S228, L235 and R409 (S228P/L235E/R409K,T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501).

In one embodiment, the antibody can comprise an Fc domain of human IgG1 origin, comprises a mutation at Kabat residue(s) 234, 235, 237, 330 and/or 331. One example of such an Fc domain comprises substitutions at Kabat residues L234, L235 and P331 (e.g., L234A/L235E/P331S or (L234F/L235E/P331S). Another example of such an Fc domain comprises substitutions at Kabat residues L234, L235, G237 and P331 (e.g., L234A/L235E/G237A/P331S). Another example of such an Fc domain comprises substitutions at Kabat residues L234, L235, G237, A330 and P331 (e.g., L234A/L235E/G237A/A330S/P331S). In one embodiment, the antibody comprises an Fc domain, optionally of human IgG1 isotype, comprising: a L234X₁ substitution, a L235X₂ substitution, and a P331X₃ substitution, wherein X₁ is any amino acid residue other than leucine, X₂ is any amino acid residue other than leucine, and X₃ is any amino acid residue other than proline; optionally wherein X₁ is an alanine or phenylalanine or a conservative substitution thereof; optionally wherein X₂ is glutamic acid or a conservative substitution thereof; optionally wherein X₃ is a serine or a conservative substitution thereof. In another embodiment, the antibody comprises an Fc domain, optionally of human IgG1 isotype, comprising: a L234X₁ substitution, a L235X₂ substitution, a G237X₄ substitution and a P331X₄ substitution, wherein X₁ is any amino acid residue other than leucine, X₂ is any amino acid residue other than leucine, X₃ is any amino acid residue other than glycine, and X₄ is any amino acid residue other than proline; optionally wherein X₁ is an alanine or phenylalanine or a conservative substitution thereof; optionally wherein X₂ is glutamic acid or a conservative substitution thereof; optionally, X₃ is alanine or a conservative substitution thereof; optionally X₄ is a serine or a conservative substitution thereof. In another embodiment, the antibody comprises an Fc domain, optionally of human IgG1 isotype, comprising: a L234X₁ substitution, a L235X₂ substitution, a G237X₄ substitution, G330X₄ substitution, and a P331X₅ substitution, wherein X₁ is any amino acid residue other than leucine, X₂ is any amino acid residue other than leucine, X₃ is any amino acid residue other than glycine, X₄ is any amino acid residue other than alanine, and X₅ is any amino acid residue other than proline; optionally wherein X₁ is an alanine or phenylalanine or a conservative substitution thereof; optionally wherein X₂ is glutamic acid or a conservative substitution thereof; optionally, X₃ is alanine or a conservative substitution thereof; optionally, X₄ is serine or a conservative substitution thereof; optionally X₅ is a serine or a conservative substitution thereof. In the shorthand notation used here, the format is: Wild type residue: Position in polypeptide: Mutant residue, wherein residue positions are indicated according to EU numbering according to Kabat.

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 42)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 43)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E F E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 44)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P S S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or a sequence at least 90%, 95% or 99% identical thereto but retaining the amino acid residues at Kabat positions 234, 235, 237 and 331 (underlined):

(SEQ ID NO: 45)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

Fc interaction abrogated ILT2 blocking antibodies will result in lack of agonist activity at ILT2. Such antibodies also result in no or low ADCC activity, meaning that an Fc interaction abrogated antibody exhibits an ADCC activity that is below 50% specific cell lysis. Preferably an antibody substantially lacks ADCC activity, e.g., the antibody exhibits an ADCC activity (specific cell lysis) that is below 5% or below 1%. Such antibodies can also result in lack of FcγR-mediated cross-linking of ILT2 at the surface of a cell (e.g., an NK cell, a T cell, a monocyte, a dendritic cell, a macrophage).

In one embodiment, the antibody has a substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330, 331 and 409 (numbering of residues in the heavy chain constant region is according to EU numbering according to Kabat). In one embodiment, the antibody comprises a substitution at residues 234, 235 and 322. In one embodiment, the antibody has a substitution at residues 234, 235 and 331. In one embodiment, the antibody has a substitution at residues 234, 235, 237 and 331. In one embodiment, the antibody has a substitution at residues 234, 235, 237, 330 and 331. In one embodiment, the Fc domain is of human IgG1 subtype. Amino acid residues are indicated according to EU numbering according to Kabat.

An anti-ILT2 antibody can be incorporated in a pharmaceutical formulation comprising in a concentration from 1 mg/ml to 500 mg/ml, wherein said formulation has a pH from 2.0 to 10.0. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g., freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In a another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing an antibody according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen. Administration of pharmaceutical compositions according to the invention may be through several routes of administration, for example, subcutaneous, intramuscular, intraperitoneal, intravenous, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Suitable antibody formulations can also be determined by examining experiences with other already developed therapeutic monoclonal antibodies. Several monoclonal antibodies have been shown to be efficient in clinical situations, such as Rituxan (Rituximab), Herceptin (Trastuzumab) Xolair (Omalizumab), Bexxar (Tositumomab), Campath (Alemtuzumab), Zevalin, Oncolym and similar formulations may be used with the antibodies of this invention. For example, a monoclonal antibody can be supplied at a concentration of 10 mg/mL in either 100 mg (10 mL) or 500 mg (50 mL) single-use vials, formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. In another embodiment, the antibody is supplied in a formulation comprising about 20 mM Na-Citrate, about 150 mM NaCl, at pH of about 6.0.

### Diagnosis and Treatment of Malignancies

Methods of treating an individual, notably a human patient, using an anti-ILT2 antibody as described herein are also provided for. In one embodiment, the invention provides for the use of an antibody as described herein in the preparation of a pharmaceutical composition for administration to a human patient. Typically, the patient suffers from, or is at risk for, cancer or an infectious disease, e.g., a bacterial or a viral disease.

For example, in one aspect, the invention provides a method of potentiating the activity (e.g. cytotoxicity towards tumor cells) and/or proliferation of ILT2-restricted leukocytes, e.g., lymphocytes, monocytes, macrophages, dendritic cells, B cells, NK cells, CD8 T cells, in a patient in need thereof, comprising the step of administering a neutralizing anti-ILT-2 antibody of the disclosure to said patient. The antibody can be for example a human or humanized anti-ILT2 antibody, which antibody reduces or prevents HLA-mediated activation of ILT2 mediated inhibitory signaling in primary NK cells and/or CD8 T cells (e.g. as determined according to the methods disclosed herein). In one embodiment, the method is directed at increasing the activity and/or number of such lymphocytes in patients having a disease in which increased lymphocyte (e.g., NK and/or CD8+ T cell) activity is beneficial, which involves, affects or is caused by cells susceptible to lysis by NK or CD8+ T cells, or which is caused, exacerbated perpetuated or otherwise characterized by insufficient NK or CD8+ T cell activity, such as a cancer or an infectious disease.

In one embodiment, the antibodies of the disclosure are used in the treatment of a tumor characterized by expression of HLA-A2 and/or HLA-G, optionally overexpression of HLA-A2 and/or HLA-(compared to expression in, e.g., healthy tissue, in healthy individuals).

A wide range of cancers are known to be characterized by HLA-G-expressing tumor cells. For example, HLA-G+ lesions (greater than 30% of tumor cells) have been reported in cutaneous melanoma, clear cell renal carcinoma, retinoblastoma, spinous cell carcinoma, in situ carcinoma, colorectal cancer, ovarian carcinoma, cutaneous T cell lymphoma, endometrial adenocarcinoma, cutaneous B cell lymphoma, gastric cancer, ampullary cancer, bilary cancer and pancreatic ductal adenocarcinoma. HLA-G+ lesions (less than 30% of tumor cells) have also been reported in leukemia, basal cell carcinoma, bladder cancer, breast cancer, malignant mesothelioma, actinic keratosis and lung carcinoma. Furthermore, a wide range of cancers, including many cancers that express HLA-G, are known to be characterized by HLA-E-expressing tumor cells, for example non-small cell lung cancer (NSCLC)), renal cell carcinoma (RCC), melanoma, head and neck squamous cell carcinoma (HNSCC), colorectal cancer, cervical cancer and ovarian cancer are known to express HLA-E, including at high levels.

In one embodiment, anti-ILT2 antibodies are used in the treatment of a bladder cancer. In one embodiment, anti-ILT2 antibodies are used in the treatment of urothelial carcinoma. Urothelial carcinoma (also called transitional cell carcinoma) is a malignant tumour of the bladder that can spread (metastasize) to other parts of the body. Urothelial carcinoma can start in any part of the urinary tract, including the renal pelvis, ureters, bladder or urethra.

The methods and compositions herein can be utilized for the treatment of Renal Cell Carcinoma. The initial symptoms of Renal Cell Carcinoma typically include: blood in the urine (occurring in 40% of affected persons at the time that medical advice is sought); and/or flank pain (40%); and/or a mass in the abdomen or flank (25%); and/or weight loss (33%); and/or fever (20%); and/or high blood pressure (20%); and/or night sweats; and/or malaise. Renal Cell Carcinoma is also typically associated with a number of “paraneoplastic syndromes”, which are conditions caused by either the hormones produced by the tumour itself or by the body's attack on the tumour, and which commonly affect tissues which do not actually house the tumour. The most common syndromes are selected from: anaemia or polycythaemia; and/or high blood calcium levels; and/or thrombocytosis; and/or secondary amyloidosis.

It will be appreciated that Renal Cell Carcinoma is a general term that encompasses a range of distinct types of RCC, including: metastatic clear cell RCC; localised clear cell RCC; multilocular cystic clear cell RCC; tubulocystic RCC; thyroid-like follicular RCC; acquired cystic kidney disease-associated RCC; hybrid oncocytoma/chromophobe RCC. Thus, in one embodiment, the methods and compositions herein are used to treat a metastatic clear cell RCC. In one embodiment, the methods and compositions herein are used to treat a localised clear cell RCC. In one embodiment, the methods and compositions herein are used to treat a multilocular cystic clear cell RCC. In one embodiment, the methods and compositions herein are used to treat a tubulocystic RCC. In one embodiment, the methods and compositions herein are used to treat a thyroid-like follicular RCC. In one embodiment, the methods and compositions herein are used to treat an acquired cystic kidney disease-associated RCC. In one embodiment, the methods and compositions herein are used to treat a hybrid oncocytoma/chromophobe RCC.

An individual can be treated with an anti-ILT2 antibody with or without a prior detection step to assess expression of HLA-A2 and/or HLA-G (and/or HLA-E) on the surface of tumor cells. A tumor or cancer may in one aspect be a type of tumor or cancer that is known to be generally characterized by expression of HLA-A2 and/or HLA-G (and optionally further HLA-E) (or of one or more other natural ligands of ILT2). In some embodiments, treatment methods can comprise a step of detecting a HLA-A2 and/or HLA-G (and optionally further HLA-E) nucleic acid or polypeptide in a biological sample of a tumor (e.g. on a tumor cell) from an individual. A determination that a biological sample expresses HLA-A2 and/or HLA-G (and optionally further HLA-E), e.g. expresses HLA-A2 and/or HLA-G (and optionally further HLA-E) at a detectable level, expresses HLA-A2 and/or HLA-G (and optionally further HLA-E) at least at a predetermined level, expresses HLA-A2 and/or HLA-G (and optionally further HLA-E) at a high level, or at a high intensity of staining with an anti-HLA-A2 and/or an anti-HLA-G (and/or an anti-HLA-E) antibody, in each case optionally compared to a reference) can be used to designate a patient as having a cancer that may have a particularly strong benefit from treatment with an agent that neutralizes the activity of ILT2. In one embodiment, the method comprises determining the level of expression of a HLA-A2 and/or HLA-G (and optionally further HLA-E) nucleic acid or polypeptide in a biological sample and comparing the level to a reference level (e.g. a value, strong cell surface staining, etc.) corresponding to an individual that benefits from treatment with an agent that inhibits neutralizes the activity of ILT2. A determination that a biological sample expresses HLA-G and/or HLA-A2 (and optionally further HLA-E) nucleic acid or polypeptide at a level that corresponds and/or is increased to the reference level indicates that the individual has a cancer that can have a particularly strong benefit from being treated with an agent that inhibits neutralizes the activity of ILT2. Optionally, detecting a HLA-A2 and/or HLA-G (and optionally further HLA-E) polypeptide in a biological sample comprises detecting HLA-A2 and/or HLA-G (and optionally further HLA-E) polypeptide expressed on the surface of a malignant cell.

In one embodiment of any of the cancer treatment or prevention methods herein, the treatment or prevention of a cancer in an individual comprises:

a) determining whether malignant cells (e.g., tumor cells) within the individual having a cancer express a HLA class I ligand of ILT2 (e.g., HLA-A2 and/or -G), and

b) upon a determination that the ligand(s) of ILT2 are expressed by (e.g., on the surface of) malignant cells (e.g., tumor cells), administering to the individual an anti-ILT2 antibody, e.g., an antibody according to any aspect of the disclosure.

In one embodiment, a determination that a biological sample (e.g., a sample comprising tumor cells, tumor tissue and/or tumor adjacent tissue) expresses ligands of ILT2 indicates that the individual has a cancer that can be treated with and/or may receive benefit from an antibody that inhibits an ILT2 polypeptide.

In one embodiment, significant expression of ligands of ILT2 means that said ligand(s) are expressed in a substantial number of tumor cells taken from a given individual. While not bound by a precise percentage value, in some examples a ligand can be said to be expressed if detected on at least 10%, 20% 30%, 40%, 50%, or more, of the tumor cells taken from a patient (in a sample).

Determining whether an individual has cancer cells that express an HLA-G polypeptide can for example comprise obtaining a biological sample (e.g. by performing a biopsy) from the individual that comprises cancer cells, bringing said cells into contact with an antibody that binds an HLA-A2 and/or HLA-G polypeptide, and detecting whether the cells express HLA-A2 and/or HLA-G on their surface. For anti-HLA-G antibodies see, e.g., MEM-G/9 and other antibodies in Fournel et al., (2000) Tissue Antigens 55: 510-518 and WO2018/091580, the disclosures of which are incorporated herein by reference. Optionally, determining whether an individual has cancer cells that express HLA-A2 and/or HLA-G comprises conducting an immunohistochemistry assay. Optionally determining whether an individual has cancer cells that express HLA-A2 and/or HLA-G comprises conducting a flow cytometry assay.

In one embodiment, the antibodies of the disclosure are used in the treatment of an individual having significant and/or elevated levels of ILT2 expression at the surface of NK cells and/or CD8 T cells (compared to expression in, e.g., healthy tissue, in healthy individuals). An individual can be treated with an anti-ILT2 antibody with or without a prior detection step of assessing ILT2 expression at the surface of NK cells and/or CD8 T cells. A tumor or cancer may be a type of tumor or cancer that is known to be generally characterized significant and/or elevated levels of ILT2 expression at the surface of NK cells and/or CD8 T cells (e.g., HNSCC, NSCLC, RCC, ovarian cancer). In one embodiment, such a cancer is a cancer that is resistant or non-responsive to immunotherapy (e.g. treatment with an agent that inhibits a PD-1 polypeptide). In some aspects, an individual can be selected to receive treatment with an anti-ILT2 antibody upon assessment of the presence and/or levels of ILT2 expression at the surface of NK cells and/or CD8 T cells obtained from the individual (e.g. NK and/or CD8 T cells from tumor or tumor-adjacent tissue, circulating NK and/or CD8 T cells). In one aspect, an individual can be treated with an anti-ILT2 antibody in a treatment comprising a step of determining the presence (e.g., numbers) of cells in circulation or in the tumor environment that express ILT2, and/or determining the expression level of ILT2 on NK and/or CD8 T cells in circulation or in the tumor environment. Presence of elevated expression of ILT2 on NK and/or CD8 T cells, and/or elevated numbers of ILT2-expressing NK and/or CD8 T cells can indicate an individual will derive particular benefit from treatment with an anti-ILT2 antibody. Such individual can then be treated with the anti-ILT2 antibody. Elevated numbers or expression level can be determined as compared to healthy (non-cancer) control individuals or healthy (non-tumoral) control tissue.

In any aspect, treatment of a cancer in an individual may comprise:

a) determining whether the individual has NK and/or CD8 T cells in circulation and/or in tumor or tumor adjacent tissue (e.g. tumor-infiltrating cells) that are characterized by ILT2 expression, optionally wherein ILT2 expression at the cell surface is increased compared to that observed in circulation NK and/or CD8 T cells in healthy individuals, and

b) upon the determination that the individual has NK and/or CD8 T cells in circulation and/or in tumor or tumor adjacent tissue that are characterized by ILT2 expression, optionally wherein ILT2 expression at the cell surface is increased compared to that observed in circulation NK and/or CD8 T cells in healthy individuals, administering to the individual an antibody that neutralizes the inhibitory activity of human ILT2 polypeptide.

The methods and compositions herein are utilized for the treatment of a variety of other cancers and other proliferative diseases. Because these methods operate by enhancing an immune response via blockade of inhibitory receptors on lymphocytes, they are applicable to a very broad range of cancers. In one embodiment, a human patient treated with an anti-ILT2 antibody of the disclosure has liver cancer, bone cancer, pancreatic cancer, skin cancer, cancer of the head or neck (e.g., HNSCC), breast cancer, lung cancer, non-small cell lung cancer (NSCLC), castrate resistant prostate cancer (CRPC), melanoma, uterine cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present invention is also applicable to treatment of metastatic cancers. Patients can be tested or selected for one or more of the above described clinical attributes prior to, during or after treatment.

The antibody compositions may be used to treat individuals regardless of the allele present in an individual, e.g., the alleles giving rise to functional inhibitory isoforms 1, 2 and 3 of ILT2. In one embodiment, the antibody compositions are used to treat individuals expressing an ILT2 protein comprising the amino acid sequence of SEQ ID NO: 1, individuals expressing an ILT2 protein comprising the amino acid sequence of SEQ ID NO: 2, and individuals expressing an ILT2 protein comprising the amino acid sequence of SEQ ID NO: 3. Optionally, no prior assessment step is required or carried out to determine the particular allele or isoform of ILT2 expressed in an individual. In one embodiment, the same administration regimen is used to treat such individuals whose cells express a first isoform of ILT2 and individuals who express a second isoform of ILT2; the administration regimen can comprise the same mode of administration, the same dosage and the same frequency of administration irrespective of the particular allele of ILT2 expressed in an individual.

In certain aspects an anti-ILT2 antibody can be used to treat a cancer in an individual having immune effector cells characterized by one or more markers of exhaustion and/or immunosuppression.

In certain aspects an anti-ILT2 antibody (optionally in combination with a combined treatment as further described herein) can be used to treat a cancer in an individual having a poor disease prognosis for response to a an immunotherapeutic agent (e.g. an agent that inhibits a PD-1 polypeptide, an antibody that binds a tumor-associated antigen and is of human IgG1 or other isotype that mediates ADCC toward a tumor cell), for example a poor prognosis evidenced by one or more markers indicative of lack of a sufficient anti-tumor immune response, indicative of immune exhaustion, and/or indicative of immunosuppression notably a poor prognosis for response to treatment with an agent that inhibits a PD-1 polypeptide (e.g., an anti-PD-1 or anti-PDL1 antibody). An individual having a poor disease prognosis, e.g., is at a higher risk of progression, based on one or more predictive factors.

In one embodiment, a predictive factor(s) comprises presence (e.g., numbers) of cells in circulation or in the tumor environment expressing ILT2, and/or expression levels of ILT2 on NK and/or CD8 T cells in circulation or in the tumor environment. Presence of elevated expression of ILT2 on NK and/or CD8 T cells, and/or elevated numbers of ILT2-expressing NK and/or CD8 T cells can indicate an individual has a poor prognosis for response to treatment with an antibody that inhibits a PD-1 polypeptide.

In one aspect, an anti-ILT2 antibody can be used to treat a cancer (e.g. a head and neck cancer, a lung cancer, a renal cell cancer, a bladder cancer, an HNSCC, a NSCLC, a CCRCC, a UCC) in an individual who has a poor prognosis for response to an agent (e.g., an antibody) that inhibits the PD-1 axis, or who is a non-responder, or who has experienced a partial or an incomplete response to treatment with an agent (e.g., an antibody) that inhibits the PD-1 axis, and/or whose disease has progressed following treatment with an agent (e.g., an antibody) that inhibits the PD-1 axis. In one embodiment, the individual is treated with an anti-ILT2 antibody without combined treatment with an agent that inhibits the PD-1 axis (e.g., as anti-ILT2 monotherapy, or a combination of anti-ILT2 antibody and a second therapeutic agent other than an agent inhibits the PD-1 axis). In another embodiment, the individual is treated with an anti-ILT2 antibody in combination with an agent that inhibits the PD-1 axis.

In certain aspects an anti-ILT2 antibody (optionally in combination with a combined treatment as further described herein) can be used to treat a cancer in an individual having a poor disease prognosis for response to a an immunotherapeutic agent (e.g. an agent that inhibits a PD-1 polypeptide, an antibody that binds a tumor-associated antigen and is of human IgG1 or other isotype that mediates ADCC toward a tumor cell), for example a poor prognosis evidenced by one or more markers indicative of lack of a sufficient anti-tumor immune response, indicative of immune exhaustion, and/or indicative of immunosuppression notably a poor prognosis for response to treatment with an agent that inhibits a PD-1 polypeptide (e.g., an anti-PD-1 or anti-PDL1 antibody). An individual having a poor disease prognosis, e.g., is at a higher risk of progression, based on one or more predictive factors.

In one embodiment, a predictive factor(s) comprises presence (e.g., numbers) of cells in circulation or in the tumor environment expressing ILT2, and/or expression levels of ILT2 on NK and/or CD8 T cells in circulation or in the tumor environment. Presence of elevated expression of ILT2 on NK and/or CD8 T cells, and/or elevated numbers of ILT2-expressing NK and/or CD8 T cells can indicate an individual has a poor prognosis for response to treatment with an antibody that inhibits a PD-1 polypeptide.

In any aspect, treatment of a cancer in an individual may comprise:

(a) determining whether an individual has a cancer that has responded to treatment during a prior treatment with an agent that inhibits a human PD-1 polypeptide but that has recurred or progressed,

b) upon the determination that the individual has a cancer that has responded to treatment during a prior treatment with an agent that inhibits a human PD-1 polypeptide but that has recurred or progressed, administering to the individual: an agent, optionally an antibody, that neutralizes the inhibitory activity of human ILT2 polypeptide, optionally further in combination with an agent that inhibits a human PD-1 polypeptide.

In any aspect, treatment of a cancer in an individual may comprise:

a) determining whether an individual has a cancer that is resistant to treatment with an agent that inhibits a human PD-1 polypeptide, and

b) upon the determination that the individual has a cancer that is resistant to treatment with an agent that inhibits a human PD-1 polypeptide, administering to the individual: an agent, optionally an antibody, that neutralizes the inhibitory activity of human ILT2 polypeptide (e.g. in human primary NK and/or CD8 T cells, optionally in combination with an agent that inhibits a human PD-1 polypeptide.

The anti-ILT2 antibodies may be used in as monotherapy or in combined treatments with one or more other and/or therapeutic agents. The additional therapy or therapeutic agent will normally be administered in amounts and treatment regimens typically used for that agent in a monotherapy for the particular disease or condition being treated. Such therapeutic agents include, but are not limited to anti-cancer agents and chemotherapeutic agents.

In another aspect, provided is a method of reducing the risk of cancer progression, reducing the risk of further cancer progression in a cell population that has undergone initiation, and/or providing a therapeutic regimen for reducing cancer progression in a human patient, which comprises administering to the patient one or more first treatments (e.g. induction therapy, such as a chemotherapeutic agent) in an amount and regimen sufficient to achieve a response (partial or complete response), and then administering an amount of an anti-ILT2 antibody or related composition (or applying a combination administration method) to the patient.

In a further aspect, provided is a method of promoting remission of cancer in a mammalian host, such as a human patient, comprising administering a composition comprising an anti-ILT2 antibody, to the host, so as to promote cancer remission in the host.

In an even further aspect, provided is a method for reducing the risk of developing a cancer (e.g. a metastatic or advanced cancer), reducing the time to onset of a cancerous condition, and/or reducing the severity of a cancer diagnosed in the early stages, comprising administering to a host a prophylactically effective amount of an anti-ILT2 antibody or related composition so as to achieve the desired physiological effect(s).

In a further aspect, provided is a method of increasing the likelihood of survival over a relevant period in a human patient diagnosed with a cancer (e.g. a head and neck cancer, a lung cancer, a renal cell cancer, a bladder cancer, an HNSCC, a NSCLC, a CCRCC, a UCC). In another aspect, provided is a method for improving the quality of life of a cancer patient comprising administering to the patient a composition in an amount effective to improve the quality of life thereof. In a further aspect, methods described herein can be applied to significantly reduce the number of cancer cells in a vertebrate host, such that, for example, the total number of cancer cells is reduced. In a related sense, provided is a method for killing (e.g., either directly or indirectly causing death of) cancer cells in a vertebrate, such as a human cancer patient.

In one embodiment, the anti-ILT2 neutralizing antibodies lack binding to human CD16 yet potentiate the activity of CD16-expressing effector cells (e.g., NK or effector T cells). Accordingly, in one embodiment, the anti-ILT2 compositions are used in combination with an Fc domain-containing protein capable of inducing ADCC toward a cell to which it is bound, e.g., via CD16 expressed by an NK cell. Typically, such Fc domain-containing protein is an antibody that binds to an antigen of interest, e.g., an antigen present on a tumor cell (tumor antigen) and comprises an Fc domain or portion thereof, and will exhibit binding to the antigen via the antigen binding domain and to Fcγ receptors (e.g., CD16) via the Fc domain. Tumor antigens are well known in the art, for example Receptor Tyrosine Kinase-like Orphan Receptor 1 (ROR1), B7-H3, B7-H4, B7-H6, Crypto, CD4, CD20, CD30, CD19, CD38, CD47, EGFR, Her2 (ErbB2/Neu), CD22, CD33, CD79, CD123, CD138, CD171, PSCA, PSMA, BCMA, CD52, CD56, CD80, CD70 and CD123. In one embodiment, its ADCC activity will be mediated at least in part by CD16. In one embodiment, the additional therapeutic agent is an antibody having a native or modified human Fc domain, for example an Fc domain from a human IgG1 or IgG3 antibody. The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, DC and eosinophils. The term “ADCC-inducing antibody” refers to an antibody that demonstrates ADCC as measured by assay(s) known to those of skill in the art. Such activity is typically characterized by the binding of the Fc region with various FcRs. Without being limited by any particular mechanism, those of skill in the art will recognize that the ability of an antibody to demonstrate ADCC can be, for example, by virtue of its subclass (such as IgG1 or IgG3), by mutations introduced into the Fc region, or by virtue of modifications to the carbohydrate patterns in the Fc region of the antibody. Examples of antibodies that induce ADCC include rituximab (for the treatment of lymphomas, CLL, trastuzumab (for the treatment of breast cancer), alemtuzumab (for the treatment of chronic lymphocytic leukemia) and cetuximab (for the treatment of colorectal cancer, head and neck squamous cell carcinoma), daratumumab, drozitumab, duligotumab, enoticumab, ganitumab, necitumumab, ofatumumab, panitumumab, patritumab, pritumumab, ramucirumab, and pertuzumab. Examples of ADCC-enhanced antibodies include but are not limited to: GA-101 (hypofucosylated anti-CD20), margetuximab (Fc enhanced anti-HER2), mepolizumab, MEDI-551 (Fc engineered anti-CD19), obinutuzumab (glyco-engineered/hypofucosuylated anti-CD20), ocaratuzumab (Fc engineered anti-CD20), XmAb®5574/MOR208 (Fc engineered anti-CD19). In other aspects, a treatment or use may optionally be specified as not being in combination with (or excluding treatment with) an antibody or other agent that binds CD16 and/or is capable of inducing ADCC toward a cell to which it is bound.

In another embodiment, the anti-ILT2 neutralizing antibodies can be advantageously used in combination with an agent that neutralizes the inhibitory activity of human PD-1, e.g., that inhibits the interaction between PD-1 and PD-L1, optionally further in individuals who are poor responders to (or not sensitive to) treatment with an agent that neutralizes the inhibitory activity of human PD-1. The anti-ILT2 neutralizing antibodies may be useful to potentiate the activity of PD-1-expressing effector cells (e.g., NK or effector T cells, e.g., ILT2 expressing NK cells). Accordingly, in one embodiment, the second or additional second therapeutic agent is an antibody or other agent that neutralizes the inhibitory activity of human PD-1. Examples of agents or antibodies that neutralize the inhibitory activity of human PD-1 include antibodies that bind PD1 or PD-L1. Many such antibodies are known and can be used, for example, at the exemplary the doses and/or frequencies that such agents are typically used. In one embodiment, the second or additional second therapeutic agent is an agent (e.g., an antibody) that inhibits the PD-1 axis (i.e. inhibits PD-1 or PD-L1). Antibodies that bind PD1 or PD-L1 can be used, for example, at the exemplary the doses and/or frequencies that such agents are used as monotherapy, e.g., as described below.

PD-1 is an inhibitory member of the CD28 family of receptors that also includes CD28, CTLA-4, ICOS and BTLA. PD-1 is expressed on activated B cells, T cells, and myeloid cells Okazaki et al. (2002) Curr. Opin. Immunol. 14: 391779-82; Bennett et al. (2003) J Immunol 170: 711-8). Two ligands for PD-1 have been identified, PD-L1 and PD-L2, that have been shown to downregulate T cell activation upon binding to PD-1 (Freeman et al. (2000) J Exp Med 192: 1027-34; Latchman et al. (2001) Nat Immunol 2: 261-8; Carter et al. (2002) Eur J Immunol 32: 634-43). PD-L1 is abundant in a variety of human cancers (Dong et al. (2002) Nat. Med. 8: 787-9). The interaction between PD-1 and PD-L1 results in a decrease in tumor infiltrating lymphocytes, a decrease in T-cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well. Blockade of PD-1 can advantageously involve use of an antibody that prevents PD-L1-induced PD-1 signaling, e.g. by blocking the interaction with its natural ligand PD-L1. In one aspect the antibody binds PD-1 (an anti-PD-1 antibody); such antibody may block the interaction between PD-1 and PD-L1 and/or between PD-1 and PD-L2. In another aspect the antibody binds PD-L1 (an anti-PD-L1 antibody) and blocks the interaction between PD-1 and PD-L1.

There are currently at least six agents blocking the PD-1/PD-L1 pathway that are marketed or in clinical evaluation, any of these may be useful in combination with the anti-ILT2 antibodies of the disclosure. One agent is BMS-936558 (Nivolumab/ONO-4538, Bristol-Myers Squibb; formerly MDX-1106). Nivolumab, (Trade name Opdivo®) is an FDA-approved fully human IgG4 anti-PD-L1 mAb that inhibits the binding of the PD-L1 ligand to both PD-1 and CD80 and is described as antibody 5C4 in WO 2006/121,168, the disclosure of which is incorporated herein by reference. For melanoma patients, the most significant OR was observed at a dose of 3 mg/kg, while for other cancer types it was at 10 mg/kg. Nivolumab is generally dosed at 10 mg/kg every 3 weeks until cancer progression. Another agent is durvalumab (Imfinzi®, MEDI-4736), an anti-PD-L1 developed by AstraZeneca/Medimmune and described in WO2011/066389 and US2013/034559. Another agent is MK-3475 (human IgG4 anti-PD1 mAb from Merck), also referred to as lambrolizumab or pembrolizumab (Trade name Keytruda®) has been approved by the FDA for the treatment of melanoma and is being tested in other cancers. Pembrolizumab was tested at 2 mg/kg or 10 mg/kg every 2 or 3 weeks until disease progression. Another agent is atezolizumab (Tecentriq®, MPDL3280A/RG7446, Roche/Genentech), a human anti-PD-L1 mAb that contains an engineered Fc domain designed to optimize efficacy and safety by minimizing FcγR binding and consequential antibody-dependent cellular cytotoxicity (ADCC). Doses of ≥1, 10, 15, and 25 mg/kg MPDL3280A were administered every 3 weeks for up to 1 year. In phase 3 trial, MPDL3280A is administered at 1200 mg by intravenous infusion every three weeks in NSCLC. In other aspects, a treatment or use may optionally be specified as not being in combination with (or excluding treatment with) an antibody or other agent that inhibits the PD-1 axis.

In the treatment methods, the anti-ILT2 antibody and the second therapeutic agent can be administered separately, together or sequentially, or in a cocktail. In some embodiments, the antigen-binding compound is administered prior to the administration of the second therapeutic agent. For example, the anti-ILT2 antibody can be administered approximately 0 to 30 days prior to the administration of the second therapeutic agent. In some embodiments, a ILT2-binding compound is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, an anti-ILT2 antibody is administered concurrently with the administration of the therapeutic agents. In some embodiments, an anti-ILT2 antibody is administered after the administration of the second therapeutic agent. For example, an anti-ILT2 antibody can be administered approximately 0 to 30 days after the administration of the second therapeutic agent. In some embodiments, an anti-ILT antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

In other aspects, methods are provided for identifying ILT2+ cells using the antibodies of the disclosure. Assessing the co-expression of ILT2 on cells (e.g., monocytes, DC, macrophages, NK cell, T cells) can be used in diagnostic or prognostic methods. For example, a biological sample can be obtained from an individual (e.g., from a blood sample, from cancer or cancer-adjacent tissue obtained from a cancer patient) and analyzed for the presence of ILT2+ cells. The expression of ILT2 on such cells can, for example, be used to identify individuals having such cells, for example tumor infiltrating NK and/or T cells which are inhibited by ILT2 polypeptides. The expression of ILT2 on such cells can, for example, be used to identify individuals having immune cells (e.g., NK cells and/or CD8 T cells), for example in the tumor or tumor environment which are inhibited by ILT2 polypeptides. The method can, for example, be useful as a prognostic for response to treatment with an agent that neutralizes ILT2. Expression of ILT2 on such cells can indicate an individual suitable for treatment with an antibody of the disclosure as further discussed herein.

### EXAMPLES

### Example 1: ILT2 (LILRB1) is Expressed on Healthy Human Donor Memory CD8 T Cells and CD56dim NK Cells

LILRB1 expression on peripheral blood mononuclear cells was determined by flow cytometry on fresh whole blood from healthy human donors. The NK population was determined as CD3-CD56+ cells (anti CD3 AF700-BioLegend #300424; anti CD56 BV421-BD Biosciences #740076). Among NK cells, CD56 bright subset was identify as CD16-cells whereas CD56dim subset as CD16+ cells (anti CD16 BV650-BD Biosciences #563691). CD4+ and CD8+ T cells were identify as CD3+CD56-CD4+ and CD3+CD56-CD8+ cells, respectively (CD3-see above; CD4 BV510-BD Biosciences #740161; CD8 BUV737-BD Biosciences #564629). Among the CD4+ T cell population, Tconv and Treg were identify as CD127+CD25-/low and CD127lowCD25high cells, respectively (CD127 PE-Cy7-BD Biosciences #560822; CD25 VioBright-Miltenyi Biotec #130-104-274). Among the CD8+ T cell population, the naïve, central memory, effector memory and effector memory T cell populations were identify as CD45RA+CCR7+, CD45RA-CCR7+, CD45RA-CCR7-, CD45RA+CCR7-cells, respectively (CD45RA BUV395-BD Biosciences #740298; CCR7 PerCP-Cy5.5-BioLegend #353220). A population named “CD3+CD56+ ly” was an heterogeneous cell population comprising NKT cells and γδ T cells. Monocytes were identify as CD3-CD56-CD14+ cells (CD14 BV786-BD Biosciences #563691) and B cells as CD3-CD56-CD19+ cells (CD19 BUV496-BD Biosciences #564655). Anti-LILRB1 antibody (clone HP—F1-APC-BioLegend #17-5129-42) as used. Whole blood was incubated 20 min at RT in the dark with staining Ab mix then red blood cells were lyzed with Optilyse C (Beckman Coulter #A11895) following the provider TDS. Cells were washed twice with PBS and fluorescence was revealed with Fortessa flow cytometer (BD Biosciences).

Results are shown in FIG. 1. While B lymphocytes and monocytes generally always express ILT2, conventional CD4 T cells and CD4 Treg cells did not express ILT2, but a significant fraction of CD8 T cells (about 25%), CD3+ CD56+ lymphocytes (about 50%) and NK cells (about 30%) expressed ILT2, suggesting that a proportion of each of such CD8 T and NK cell populations can be inhibited by ILT2, as a function of the HLA class I ligands present, for example on tumor cells.

Among the CD8 T cells, ILT2 expression was not present on naïve cells, but was present in effector memory fraction of CD8 T cells, and to a lesser extent, central memory CD8 T cells. Among the NK cells, the ILT2 expression was essentially only on the CD16+ subset (CD56dim), and much less frequently on CD16-NK cells (CD56bright).

### Example 2: ILT2 is Upregulated in Multiple Human Cancers

ILT2 expression on monocytes, B cells, CD4+ T cells, CD8+ T cells and both CD16− and CD16+ NK cells was determined by flow cytometry on peripheral blood mononuclear cells (PBMC) purified from whole blood of human cancer patient donors. Cell populations were identified and ILT2 expression was assessed using the same antibody mix detailed in example 1. PBMC were incubated 20 min at 4° C. in the dark with the antibody mix, wash twice in staining buffer and fluorescence was measured on a Fortessa flow cytometer.

Results from the cancer patient samples are shown in FIG. 2. As can be seen, ILT2 was once again expressed on all monocytes and B cells. However on the lymphocyte subsets, NK cells and CD8 T cells, ILT2 was expressed more frequently with statistical significance on cells from three types of cancers, HNSCC, NSCLC and RCC. ILT2 was upregulated also in ovarian cancer although greater numbers of patient samples need to be studied. This increased expression of ILT2 in cancer patient samples was observed in CD8 T cells, γδ T cells (no expression on αβ T cells) and CD16+NK cells, in head and neck cancer (HNSCC), lung cancer (NSCLC) and kidney cancer (RCC).

### Example 3: Generation of Anti-ILT2 Antibodies

### Materials and Methods

### Cloning and Production of the ILT-2_6×his Recombinant Protein

The ILT-2 protein (Uniprot access number Q8NHL6) was cloned into the pTT-5 vector between the NruI and BamHI restriction sites. A heavy chain peptide leader was used. The PCR were performed with the following primers:

(SEQ ID NO: 57)
ILT-2_For_ACAGGCGTGCATTCGGGGCACCTCCCCAAGCCCAC,
(SEQ ID NO: 58)
ILT-2_Rev_CGAGGTCGGGGGATCCTCAATGGTGGTGATGATGGTGGTG
CCTTCCCAGACCACTCTG,

A 6×His tag was added at the C-terminal part of the protein for purification. The EXPI293 cell line was transfected with the generated vector for transient production. The protein was purified from the supernatant using Ni-NTA beads and monomers were purified using a SEC.

The amino acid sequence for the ILT-2_6×His recombinant protein is shown below:

(SEQ ID NO: 59)
GHLPKPTLWAEPGSVITQGSPVTLRCQGGQETQEYRLYREKKTALWITRI
PQELVKKGQFPIPSITWEHAGRYRCYYGSDTAGRSESSDPLELVVTGAYI
KPTLSAQPSPVVNSGGNVILQCDSQVAFDGFSLCKEGEDEHPQCLNSQPH
ARGSSRAIFSVGPVSPSRRWWYRCYAYDSNSPYEWSLPSDLLELLVLGVS
KKPSLSVQPGPIVAPEETLTLQCGSDAGYNRFVLYKDGERDFLQLAGAQP
QAGLSQANFTLGPVSRSYGGQYRCYGAHNLSSEWSAPSDPLDILIAGQFY
DRVSLSVQPGPTVASGENVTLLCQSQGWMQTFLLTKEGAADDPWRLRSTY
QSQKYQAEFPMGPVTSAHAGTYRCYGSQSSKPYLLTHPSDPLELVVSGPS
GGPSSPTTGPTSTSGPEDQPLTPTGSDPQSGLGRHHHHHHH

### Generation of CHO and KHYG Cell Lines Expressing ILT Family Members at the Cell Surface

The complete forms of ILT-2 were amplified by PCR using the following primers: ILT-2_For ACAGGCGTGCATTCGGGGCACCTCCCCAAGCCC (SEQ ID NO: 60), and ILT-2_Rev_CCGCCCCGACTCTAGACTAGTGGATGGCCAGAGTGG (SEQ ID NO: 61). The PCR products were inserted into the expression vector at appropriate restriction sites. A heavy chain peptide leader was used. The vectors were then transfected into the CHO and KHYG cell lines to obtain stable clones expressing the ILT-2 protein at the cell surface. These cells were then used for hybridoma screening. CHO cells expressing other ILT family members were prepared similarly, including cells expressing ILT-1, ILT-3, ILT-4, ILT-5, ILT-6, ILT7 and ILT-8. The amino acid sequences of the ILT proteins used to prepare the ILT-1, ILT-3, ILT-4, ILT-5 and ILT-6-expressing cells are provided in Table 4 below.

### Generation of K562 Cell Line Expressing HLA-G at the Cell Surface

The complete forms of HLA-G (Genbank access number NP_002118.1, sequence shown below) was amplified by PCR using the following primers: HLA-G_For 5′ CCAGAACACAGGATCCGCCGCCACCATGGTGGTCATGGCGCCC 3′ (SEQ ID NO: 62), HLA-G_Rev_5′ TTTTCTAGGTCTCGAGTCAATCTGAGCTCTTCTTTC 3′ (SEQ ID NO: 63). The PCR products were inserted into a vector between the BamHI and XhoI restriction sites and used to transduce K562 cell lines which either did not express HLA-E or were engineered to stably overexpress HLA-E.

| HLA-G amino acid sequence: | |
|---|---|
| (SEQ ID NO: 10) | |
| 1 | MVVMAPRTLF LLLSGALTLT ETWAGSHSMR YFSAAVSRPG RGEPRFIAMG YVDDTQFVRF |
| 61 | DSDSACPRME PRAPWVEQEG PEYWEEETRN TKAHAQTDRM NLQTLRGYYN QSEASSHTLQ |
| 121 | WMIGCDLGSD GRLLRGYEQY AYDGKDYLAL NEDLRSWTAA DTAAQISKRK CEAANVAEQR |
| 181 | RAYLEGTCVE WLHRYLENGK EMLQRADPPK THVTHHPVFD YEATLRCWAL GFYPAEIILT |
| 241 | WQRDGEDQTQ DVELVETRPA GDGTFQKWAA VVVPSGEEQR YTCHVQHEGL PEPLMLRWKQ |
| 301 | SSLPTIPIMG IVAGLVVLAA VVTGAAVAAV LWRKKSSD |

HLA-E amino acid sequence (Uniprot P13747):
(SEQ ID NO: 11)
MVDGTLLLLL SEALALTQTW AGSHSLKYFH TSVSRPGRGE PRFISVGYVD
DTQFVRFDND AASPRMVPRA PWMEQEGSEY WDRETRSARD TAQIFRVNLR
TLRGYYNQSE AGSHTLQWMH GCELGPDGRF LRGYEQFAYD GKDYLTLNED
LRSWTAVDTA AQISEQKSND ASEAEHQRAY LEDTCVEWLH KYLEKGKETL
LHLEPPKTHV THHPISDHEA TLRCWALGFY PAEITLTWQQ DGEGHTQDTE
LVETRPAGDG TFQKWAAVVV PSGEEQRYTC HVQHEGLPEP VTLRWKPASQ
PTIPIVGIIA GLVLLGSVVS GAVVAAVIWR KKSSGGKGGS YSKAEWSDSA
QGSESHSL

### Immunization and Screening

An immunization was performed by immunizing balb/c mice with ILT-2_6×His protein. After the immunization protocol the mice were sacrificed to perform fusions and get hybridomas. The hybridoma supernatants were used to stain CHO-ILT2 and CHO-ILT4 cell lines to check for monoclonal antibody reactivities in a flow cytometry experiment. Briefly, the cells were incubated with 50 μl of supernatant for 1H at 4° C., washed three times and a secondary antibody Goat anti-mouse IgG Fc specific antibody coupled to AF647 was used (Jackson Immunoresearch, JI115-606-071). After 30 min of staining, the cells were washed three times and analyzed using a FACS CANTO II (Becton Dickinson).

About 1500 hybridoma supernatants were screened, to identify those producing antibodies that bind to ILT2 and have the ability to block the interaction between ILT2 with HLA-G. Briefly, recombinant 6×HIS tagged ILT2 was incubated with 50 μl of hybridoma supernatant for 20 min at RT prior incubation with 10⁵ K562 cells expressing HLA-G. Then, cells were washed once and incubated with a secondary complex made of rabbit anti-6×HIS (Bethyl lab, A190-214A) antibody and anti-rabbit IgG F(ab′)² antibody coupled to PE (Jackson lab, 111-116-114). After 30 min of staining, the cells were washed once in PBS and fixed with Cell Fix (Becton Dickinson, 340181). Analysis was performed on a FACS CANTO II flow cytometer.

This assays permitted the identification of a panel of anti-ILT2 antibodies that were highly effective in blocking the interaction of ILT2 with its HLA class I ligand HLA-G. Antibodies 3H5, 12D12, 26D8, 18E1, 27C10, 27H5, 1C11, 1D6, 9G1, 19F10a and 27G10 were identified as having good blocking activity and thus selected for further study.

The resulting antibodies were produced as modified human IgG1 antibodies having heavy chains with Fc domain mutations L234A/L235E/G237A/A330S/P331S (Kabat EU numbering) which resulted in lack of binding to human Fcγ receptors CD16A, CD16B, CD32A, CD32B and CD64. These Fc domain mutated L234A/L235E/G237A/A330S/P331S antibodies were then used in all the other experiments described herein. Briefly, the VH and Vk sequences of each antibody (the VH and Vk variable regions shown in herein) were cloned into expression vectors containing the hulgG1 constant domains harboring the aforementioned mutations and the huCk constant domain respectively. The two obtained vectors were co-transfected into the CHO cell line. The established pool of cell was used to produce the antibody in the CHO medium.

### Example 4: Binding of Modified Human IgG1 Fc Domains to FcγR

The L234A/L235E/G237A/A330S/P331S Fc domains employed in Example 3, as well as other Fc mutations and wild-type antibodies, were previously evaluated to assess binding to human Fcγ receptors, as follows.

SPR (Surface Plasmon Resonance) measurements were performed on a Biacore T100 apparatus (Biacore GE Healthcare) at 25° C. In all Biacore experiments HBS-EP+ (Biacore GE Healthcare) and 10 mM NaOH, 500 mM NaCl served as running buffer and regeneration buffer respectively. Sensorgrams were analyzed with Biacore T100 Evaluation software. Recombinant human FcR's (CD64, CD32a, CD32b, CD16a and CD16b) were cloned, produced and purified.

Antibodies tested included: antibodies having wild type human IgG1 domain, antibodies having a human IgG4 domain with S241P substitution, human IgG1 antibodies having a N297S substitution, human IgG1 antibodies having L234F/L235E/P331S substitutions, human IgG1 antibodies having L234A/L235E/P331S substitutions, human IgG1 antibodies having L234A/L235E/G237A/A330S/P331S substitutions, and human IgG1 antibodies having L234A/L235E/G237A/P331S substitutions.

Antibodies were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Antibodies were diluted to 10 μg/ml in coupling buffer (10 mM acetate, pH 5.6) and injected until the appropriate immobilization level was reached (i.e. 800 to 900 RU). Deactivation of the remaining activated groups was performed using 100 mM ethanolamine pH 8 (Biacore GE Healthcare).

Monovalent affinity study was assessed following a classical kinetic wizard (as recommended by the manufacturer). Serial dilutions of soluble analytes (FcRs) ranging from 0.7 to 60 nM for CD64 and from 60 to 5000 nM for all the other FcRs were injected over the immobilized bispecific antibodies and allowed to dissociate for 10 min before regeneration. The entire sensorgram sets were fitted using the 1:1 kinetic binding model for CD64 and with the Steady State Affinity model for all the other FcRs.

The results are shown in Table 7, below. Results showed that while full length wild type human IgG1 bound to all human Fcγ receptors, and human IgG4 in particular bound significantly to FcγRI (CD64) (KD shown in Table 7), the L234A/L235E/G237A/A330S/P331S substitutions and L234A/L235E/G237A/P331S substitutions abolished binding to CD64 as well as to CD16a.

### Example 5: Ability of ILT2 Blocking Antibodies to Enhance NK Cell Lysis

The ability of the anti-ILT2 antibodies to control ILT2-mediated inhibition of NK cell activation was determined by the capacity of ILT2-expressing KHYG cells described in Example 3 to lyse target cells in presence of antibodies. Effector cells were KHYG cells expressing ILT2 and GFP as control and target cells were ⁵¹Cr loaded K562 cell line (ATCC® CCL-243™) made to express HLA-G. Effector and target cells were mixed at a ratio 1:10. Antibodies were pre-incubated 30 minutes at 37° C. with effector cells and then target cells were co-incubated 4 hours at 37° C. Specific lysis of target cells was calculated by the release of ⁵¹Cr in co-culture supernatant with a TopCount NXT (Perkin Elmer).

This experiment evaluated antibodies 3H5, 12D12, 26D8, 18E1, 27C10, 27H5, 1C11, 1D6, 9G1, 19F10a, 27G10 identified in Example 2, as well as commercially available antibodies GHI/75 (mouse IgG2b, Biolegend #333720), 292319 (mouse IgG2b, Bio-Techne #MAB20172), HP—F1 (mouse IgG1, eBioscience #16-5129-82), 586326 (mouse IgG2b, Bio-Techne #MAB30851) and 292305 (mouse IgG1, Bio-Techne #MAB20171).

Results are shown in FIG. 3. Most of the ILT2/HLA-G blocking antibodies showed a significant increase in % cytotoxicity by the NK cell lines toward the K562-HLA-G tumor target cells. However, certain antibodies were particular potent at increasing NK cell cytotoxicity. Antibodies 12D12, 19F10a and commercial 292319 were significantly more effective than other antibodies in the ability to enhance NK cell cytotoxicity toward the target cells. Antibodies 18E1, 26D8, although less effective, displayed activity as enhancers of cytotoxicity, followed to a lesser extent by 3H5 and commercial antibody HP—F1. Other antibodies, including 27C10, 27H5, 1C11, 1D6, 9G1 and commercial antibodies 292305, 586326, GHI/75 were considerably less active than 18E1, 26D8 in their ability to induce cytotoxicity toward target cells.

### Example 6: Blockade of ILT2 Binding to HLA Class I Molecules

### HLA/ILT2 Blocking Assay

Ability of anti-ILT2 antibodies to block the interactions between HLA-G or HLA-A2 expressed at the surface of cell lines and recombinant ILT2 protein was assessed by flow cytometry. Briefly, BirA-tagged ILT2 protein was biotinylated to obtain 1 biotin molecule per ILT2 protein. APC-conjugated streptavidin (SA) was mixed with Biotinylated ILT2 protein (ratio 1 Streptavidin per 4 ILT2 protein) to form tetramers. Anti-ILT2 Abs (12D12, 18E1, 26D8) were incubated at 4° C. in staining buffer for 30 min with ILT2-SA tetramers. The Ab-ILT2-SA complexes were added on HLA-G or HLA-A2 expressing cells and incubated for 1 hour at 4° C. The binding of complexes on cells was evaluated on a Accury C6 flow cytometer equipped with an HTFC plate loader and analyzed using the FlowJo software.

This assays permitted the identification of a panel of anti-ILT2 antibodies that were highly effective in blocking the interaction of ILT2 with its HLA class I ligand HLA-G. Antibodies 3H5, 12D12, 26D8, 18E1, 27C10, 27H5, 1C11, 1D6, 9G1, 19F10a and 27G10 all blocked ILT2 binding to HLA-G and HLA-A2. FIG. 4 shows representative results for antibodies 12D12, 18E1, and 26D8.

### Example 7: Antibody Titration on ILT2-Expressing Cells by Flow Cytometry

In order to explain the differences in NK cytotoxicity induction, unlabeled antibodies 3H5, 12D12, 26D8, 18E1, 27C10, 27H5, 1C11, 1D6, 9G1, 19F10a and 27G10 as well as the commercially available antibodies GHI/75, 292319, HP—F1, 586326 and 292305 were tested in experiments for binding to CHO cells modified to express human ILT-2. Cells were incubated with various concentrations of unlabeled anti-ILT2 antibodies from 30 μg/ml to 5×10-⁴ μg/ml, for 30 minutes at 4° C. After washes with staining buffer, cells were incubated for 30 min at 4° C. with Goat anti-human H+L AF488 secondary antibody (Jackson Immunoresearch #109-546-088) or Goat anti-mouse H+L AF488 secondary antibody for commercially available antibodies (Jackson Immuoresearch #115-545-146). Fluorescence was measured on an Accury C6 flow cytometer equipped with an HTFC plate loader.

Results are shown in Table 1, below. Except for antibody GHI/75 which had an EC50 in the range of 1-log higher that the other antibodies, the rest of the antibodies all showed comparable EC50 values, suggesting that differences binding affinity does not explain the observed differences in ability to enhance NK cell cytotoxicity.

TABLE 1

| | CHO-ILT2 | Primary NK |
|---|---|---|
| | cells EC50 | cells EC50 |
| Antibody | (μg/mL) | (μg/mL) |
| 3H5 | 0.35 | 0.48 |
| 12D12 | 0.36 | 0.09 |
| 26D8 | 0.15 | 0.11 |
| 18E1 | 0.12 | 0.11 |
| 27C10 | 0.25 | 0.33 |
| 27H5 | 0.52 | NA |
| 1C11 | 0.30 | 0.22 |
| 1D6 | 0.21 | 0.20 |
| 9G1 | 0.35 | 0.24 |
| 19F10a | 0.11 | 0.09 |
| 27G10 | 0.21 | 1.1 |
| HP-F1 | 0.56 | 0.09 |
| 292319 | 0.22 | 0.47 |
| 586326 | 0.13 | ND |
| GHI/75 | 5.39 | ND |
| 292305 | 0.27 | ND |

### Example 8: Monovalent Affinity Determination

Antibodies 3H5, 12D12, 26D8, 18E1, 27C10, 27H5, 1C11, 1D6, 9G1, 19F10a, and 27G10 as well as the commercially available antibodies GHI/75, 292319 and HP—F1 were tested for binding affinity to human ILT2 proteins.

SPR (Surface Plasmon Resonance) methods were used to test antibodies 3H5, 12D12, 26D8, 18E1, 27C10, 27H5, 1C11, 1D6, 9G1, 19F10a, 27G10 (all of human IgG1 isotype). Measurements were performed on a Biacore T200 apparatus (Biacore GE Healthcare) at 25° C. In all Biacore experiments HBS-EP+(Biacore GE Healthcare) and NaOH 10 mM served as running buffer and regeneration buffer respectively. Sensorgrams were analyzed with Biacore T100 Evaluation software. Protein-A was purchased from (GE Healthcare). Human ILT2 recombinant proteins were cloned, produced and purified at Innate Pharma. Protein-A proteins were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A was diluted to 10 μg/ml in coupling buffer (10 mM acetate, pH 5.6) and injected until the appropriate immobilization level was reached (i.e. 600 RU). Deactivation of the remaining activated groups was performed using 100 mM ethanolamine pH 8 (Biacore GE Healthcare). Anti-ILT2 antibodies at 2 μg/mL were captured onto the Protein-A chip and recombinant human ILT2 proteins were injected at different concentrations in a range from 250 nM to 1.95 nM over captured antibodies. For blank subtraction, cycles were performed again replacing ILT2 proteins with running buffer. The monovalent affinity analysis was conducted following a regular Capture-Kinetic protocol as recommended by the manufacturer (Biacore GE Healthcare kinetic wizard). Seven serial dilutions of human ILT2 proteins, ranging from 1.95 nM to 250 nM were sequentially injected over the captured antibodies and allowed to dissociate for 10 min before regeneration. The entire sensorgram sets were fitted using the 1:1 kinetic binding model or two state reaction model, as a function of the profile of the curves.

OCTET analysis was used to evaluate antibodies GHI/75, 292319 and HP—F1, (all mouse isotypes). Measurements were performed on an Octet RED96 System (Fortebio). In all Biacore experiments Kinetics Buffer 10× (Fortebio) and Glycine 10 mM pH 1.8 served as running buffer and regeneration buffer respectively. Graphs were analyzed with Data Analysis 9.0 software. Anti-Mouse IgG Fc Capture (AMC) biosensors are used. Anti-ILT2 antibodies at 5 μg/mL were captured onto Anti-Mouse IgG Fc Capture (AMC) biosensors. Seven dilutions of recombinant human ILT2 proteins were injected (from 1000 nM to 15.625 nM for 292319 and HP—F1 and from 100 nM to 1.5625 nM for GHI-75). The curves were fitted using the model 1:1

Results are shown in Table 2, below. The KD differences generally does not appear to correlate to the differences in ability to enhance NK cell cytotoxicity. Binding affinity therefore does not explain the differences in the antibodies' ability to enhance NK cell cytotoxicity.

TABLE 2

| mAb | KD (nM) | Ka (1/ms) | Kd (1/s) |
|---|---|---|---|
| 3H5 | 4.4 | ka1: | kd1: |
| | | 2.8E+5 | 8.0E−3 |
| | | ka2: | kd2: |
| | | 8.7E−4 | 1.6E−4 |
| 12D12 | 1.0 | 4.3E+5 | 4.2E−4 |
| 26D8 | 0.4 | 6.2E+5 | 2.2E−4 |
| 18E1 | 0.2 | 7.5E+5 | 1.1E−4 |
| 27C10 | 0.2 | 1.4E+5 | 3.0E−4 |
| 27H5 | 13.9 | ka1: | kd1: |
| | | 6.6E+5 | 0.1 |
| | | ka2: | kd2: |
| | | 5.3E−3 | 4.2E−4 |
| 1C11 | 0.3 | 3.4E+5 | 1.1E−4 |
| 1D6 | 0.4 | 3.2E+5 | 1.2E−4 |
| 9G1 | 0.3 | 4.0E+5 | 1.3E−4 |
| 19F10a | 5.3 | 6.6E+5 | 3.5E−3 |
| 27G10 | 0.5 | 3.5E+5 | 1.8E−4 |
| GHI/75 | 28.1 | 1.3E+4 | 3.8E−4 |
| 292319 | 0.6 | 3.0E+5 | 1.7E−4 |
| HP-F1 | 2.3 | 4.6E+5 | 1.1E−3 |

### Example 9: Identification of Antibodies that Increase Cytotoxicity in Primary Human NK Cells

We considered the possibility that the inability of prior antibodies to neutralize ILT2 in NK cells might be related to differences in ILT2 expression in primary NK cells compared for example to highly selected or modified NK cell lines that express much higher levels of ILT2 at their surface. We studied and selected antibodies in primary NK cells from a number of healthy human donors. The effect of the anti-ILT2 antibodies of Example 5 was studied by activation assays by assessing CD137 surface expression on NK cells. In each case, primary NK cells (as fresh NK cells purified from donors) were used as effector cells and K562 cells (chronic myelogenous leukemia (CML)) expressing HLA-E/G were used as targets. The targets consequently thus expressed not only the ILT2 ligand HLA-G, but also HLA-E which is an HLA class I ligand expressed on the surface of a range of cancer cells and which can interact with inhibitory receptors on the surface of NK and CD8 T cells.

Briefly, the effect of the anti-ILT2 antibodies on NK cells activation was determined by analysis by flow cytometry of CD137 expression on total NK cells, ILT2-positive NK cells and ILT2-negative NK cells. Effector cells were primary NK cells (fresh NK cells purified from donors, incubation overnight at 37° C. before use) and target cells (K562 HLA-E/G cell line) were mixed at a ratio 1:1. The CD137 assay was carried out in 96 U well plates in completed RPMI, 200 μL final/well. Antibodies were pre-incubated 30 minutes at 37° C. with effector cells and then target cells were co-incubated overnight at 37° C. The following steps were: spin 3 min at 500 g; wash twice with Staining Buffer (SB); addition of 50 μL of staining Ab mix (anti-CD3 Pacific blue-BD Biosciences; anti-CD56-PE-Vio770-Miltenyi Biotec; anti-CD137-APC-Miltenyi Biotec; anti-ILT2-PE—clone HP—F1, eBioscience); incubation 30 min at 4° C.; wash twice with SB; resuspended pellet with SB; and fluorescence revealed with Canto II (HTS). Negative controls were NK cells vs K562-HLA-E/G alone and in presence of isotype control.

FIG. 5A is a representative figure showing the increase of % of total NK cells expressing CD137 mediated by anti-ILT2 antibodies using NK cells from two human donors and K562 tumor target cells made to express HLA-E and HLA-G. FIG. 5B is a representative figure showing the increase of % of ILT2-positive (left hand panel) and ILT2-negative (right hand panel) NK cells expressing CD137 mediated anti-ILT2 antibodies using NK cells from two human donors and an HLA-A2-expressing B cell line.

Surprisingly, it was observed that antibodies that were most effective in enhancing cytotoxicity of NK cell lines were not necessarily able to activate the primary human NK cells. Among the antibodies 12D12, 19F10a and 292319 that were most effective in enhancing cytotoxicity of NK cell lines, both 19F10a and 292319 substantially lacked the ability to activate the primary NK cells all, compared to isotype control antibodies.

On the other hand, antibodies 12D12, 18E1 and 26D8 showed strong activation of the primary NK cells. Study of ILT2-positive NK cells showed that these antibodies mediated a two-fold increase in activation of the NK cells toward the target cells. As a control, % of ILT2-negative NK cells expressing CD137 were not affected by the antibodies.

FIGS. 6A and 6B shows the ability of antibodies to enhance cytotoxicity of primary NK cells toward the tumor target cells in terms of fold-increase of cytotoxicity marker CD137. FIG. 6A shows the ability of antibodies to enhance NK cell activation in presence of HLA-G-expressing target cells using primary NK cells from 5-12 different donors against HLA-G and HLA-E expressing K562 target cells. FIG. 6A shows the ability of antibodies to enhance NK cell activation in presence of HLA-G-expressing target cells using primary NK cells from 3-14 different donors against the HLA-A2 expressing target B cells. In each case 12D12, 18E1 and 26D8 had greater enhancement of NK cytotoxicity compared to one of the antibodies (292319) which was among the antibodies showing strongest enhancement of NK cytotoxicity when using NK cell lines in Example 5.

### Example 10: Characterization of Binding to ILT Family Members

To further characterize the binding specificity of the antibodies, antibodies were tested by flow cytometry for binding to the cells made to express different ILT family proteins. In addition to ILT2 (LILRB1)-expressing cells described above, cells expressing human ILT1 (LILRA2), ILT3 (LILRB4), ILT4 (LILRB2), ILT5 (LILRB3), ILT6 (LILRA3), ILT7 (LILRA4) or ILT8 (LILRA6) were generated.

The human ILT genes were amplified by PCR using the primers described in Table 3 below. The PCR product were inserted into the expression vector at appropriate restriction sites. A heavy chain peptide leader was used and a V5 tag having the amino acid sequence GKPIPNPLLGLDST (SEQ ID NO: 80) was added at the N-terminal (not shown in the sequences in Table 4). Amino acid sequences for different human ILT proteins used herein are shown below in Table 4, below. The vectors were then transfected into the CHO cell line to obtain stable clones expressing the different ILT proteins at the cell surface.

TABLE 3

| | Genbank | |
|---|---|---|
| Constructs | number | Forward primers |
| ILT-1 | NM_001130917.2 | 5′ ACAGGCGTGCATTCGGGTAAGCCTATCCCTAACCCTCTCCTCGGTC |
| | | TCGATTCTACGGGGCACCTCCCCAAGCCCACCCTCTGGGCTGAGCC 3′ |
| | | (SEQ ID NO: 64) |
| ILT-2 | Q8NHL6.1 | 5′ ACAGGCGTGCATTCGGGTAAGCCTATCCCTAACCCTCTCCTCGGTC |
| | | TCGATTCTACGGGGCACCTCCCCAAGCCCACCCTCTGGGCTGAGCC 3′ |
| | | (SEQ ID NO: 65) |
| ILT-3 | NM_001278428.3 | 5′ ACAGGCGTGCATTCGGGTAAGCCTATCCCTAACCCTCTCCTCGGTC |
| | | TCGATTCTACGGGGCCCCTCCCCAAACCCACCCTCTGGGCTGAGCCA 3′ |
| | | (SEQ ID NO: 66) |
| ILT-4 | Q8N423.4 | 5′ ACAGGCGTGCATTCGGGTAAGCCTATCCCTAACCCTCTCCTCGGTC |
| | | TCGATTCTACGGGGACCATCCCCAAGCCCACCCTGTGGGCTGAGCCA 3′ |
| | | (SEQ ID NO: 67) |
| ILT-5 | AF000575.1 | 5′ ACAGGCGTGCATTCGGGTAAGCCTATCCCTAACCCTCTCCTCGGTC |
| | | TCGATTCTACGGGGCCCTTCCCCAAACCCACCCTCTGGGCTGAGCC 3′ |
| | | (SEQ ID NO: 68) |
| ILT-6 | | 5′ ACAGGCGTGCATTCGGGTAAGCCTATCCCTAACCCTCTCCTCGGTC |
| | | TCGATTCTACGGGGCCCCTCCCCAAACCCACCCTCTGGGCTGAGCCA 3′ |
| | | (SEQ ID NO: 69) |
| ILT-7 | AF041261.1 | 5′ ACAGGCGTGCATTCGGGTAAGCCTATCCCTAACCCTCTCCTCGGTC |
| | | TCGATTCTACGGAAAACCTACCCAAACCCATCCTGTGGGCCGAGCCA 3′ |
| | | (SEQ ID NO: 70) |
| ILT-8 | AF041262.1 | 5′ ACAGGCGTGCATTCGGGTAAGCCTATCCCTAACCCTCTCCTCGGTC |
| | | TCGATTCTACGGGGCCCTTCCCCAAACCCACCCTCTGGGCTGAGCC 3′ |
| | | (SEQ ID NO: 71) |
| | Genbank | |
| Constructs | number | Reverse primers |
| ILT-1 | NM_001130917.2 | 5′ CCGCCCCGACTCTAGATCATCTCTGGCTGTGCTGAGC 3′ |
| | | (SEQ ID NO: 72) |
| ILT-2 | Q8NHL6.1 | 5′ CCGCCCCGACTCTAGACTAGTGGATGGCCAGAGTGG 3′ |
| | | (SEQ ID NO: 73) |
| ILT-3 | NM_001278428.5 | 5′ CCGCCCCGACTCTAGATCAGGCATAGACACTGGGCTC 3′ |
| | | (SEQ ID NO: 74) |
| ILT-4 | Q8N423.4 | 5′ CCGCCCCGACTCTAGACTAGTGGATGGCCAGGGTGG 3′ |
| | | (SEQ ID NO: 75) |
| ILT-5 | AF000575.1 | 5′ CCGCCCCGACTCTAGATCAGGCGTAGATGCTGGGCTC 3′ |
| | | (SEQ ID NO: 76) |
| ILT-6 | | 5′ CCGCCCCGACTCTAGATCAAGAGTAAAGATGCAGAAGACTAAGACT |
| | | GACTACAAATAGGGAAGCAGTAGATTGAAGAGCACCCTCACCAGCCTT |
| | | GGAGTCGGACTTGTTTTGTGGT 3′ |
| | | (SEQ ID NO: 77) |
| ILT-7 | AF041261.1 | 5′ CCGCCCCGACTCTAGATCACTCCACCACTCTGAAGGG 3′ |
| | | (SEQ ID NO: 78) |
| ILT-8 | AF041262.1 | 5′ CCGCCCCGACTCTAGATCAATCTTGGGGGTTTCTCTG 3′ |
| | | (SEQ ID NO: 79) |

TABLE 4
ILT sequences

| | SEQ ID | |
|---|---|---|
| Protein | NO | Sequence (AA) |
| Human | 3 | GHLPKPTLWAEPGSVIIQGSPVTLRCQGSLQAEEYHLYRENKSASWVRRIQEP |
| ILT-1 | | GKNGQFPIPSITWEHAGRYHCQYYSHNHSSEYSDPLELVVTGAYSKPTLSALP |
| | | SPVVTLGGNVTLQCVSQVAFDGFILCKEGEDEHPQRLNSHSHARGWSWAIFSV |
| | | GPVSPSRRWSYRCYAYDSNSPYVWSLPSDLLELLVPGVSKKPSLSVQPGPMVA |
| | | PGESLTLQCVSDVGYDRFVLYKEGERDFLQRPGWQPQAGLSQANFTLGPVSPS |
| | | HGGQYRCYSAHNLSSEWSAPSDPLDILITGQFYDRPSLSVQPVPTVAPGKNVT |
| | | LLCQSRGQFHTFLLTKEGAGHPPLHLRSEHQAQQNQAEFRMGPVTSAHVGTYR |
| | | CYSSLSSNPYLLSLPSDPLELVVSASLGQHPQDYTVENLIRMGVAGLVLVVLG |
| | | ILLFEAQHSQR |
| Human | 2 | GHLPKPTLWAEPGSVITQGSPVTLRCQGGQETQEYRLYREKKTAPWITRIPQE |
| ILT-2 | | LVKKGQFPIPSITWEHAGRYRCYYGSDTAGRSESSDPLELVVTGAYIKPTLSA |
| | | QPSPVVNSGGNVTLQCDSQVAFDGFILCKEGEDEHPQCLNSQPHARGSSRAIF |
| | | SVGPVSPSRRWWYRCYAYDSNSPYEWSLPSDLLELLVLGVSKKPSLSVQPGPI |
| | | VAPEETLTLQCGSDAGYNRFVLYKDGERDFLQLAGAQPQAGLSQANFTLGPVS |
| | | RSYGGQYRCYGAHNLSSEWSAPSDPLDILIAGQFYDRVSLSVQPGPTVASGEN |
| | | VTLLCQSQGWMQTFLLTKEGAADDPWRLRSTYQSQKYQAEFPMGPVTSAHAGT |
| | | YRCYGSQSSKPYLLTHPSDPLELVVSGPSGGPSSPTTGPTSTSAGPEDQPLTP |
| | | TGSDPQSGLGRHLGVVIGILVAVILLLLLLLLLFLILRHRRQGKHWTSTQRKA |
| | | DFQHPAGAVGPEPTDRGLQWRSSPAADAQEENLYAAVKHTQPEDGVEMDTRSP |
| | | HDEDPQAVTYAEVKHSRPRREMASPPSPLSGEFLDTKDRQAEEDRQMDTEAAA |
| | | SEAPQDVTYAQLHSLTLRRKATEPPPSQEGEPPAEPSIYATLAIH |
| Human | 4 | GPLPKPTLWAEPGSVISWGNSVTIWCQGTLEAREYRLDKEESPAPWDRQNPLE |
| ILT-3 | | PKNKARFSIPSMTEDYAGRYRCYYRSPVGWSQPSDPLELVMTGAYSKPTLSAL |
| | | PSPLVTSGKSVTLLCQSRSPMDTFLLIKERAAHPLLHLRSEHGAQQHQAEFPM |
| | | SPVTSVHGGTYRCFSSHGFSHYLLSHPSDPLELIVSGSLEGPRPSPTRSVSTA |
| | | GPEDQPLMPTGSVPHSGLRRHWEVLIGVLVVSILLLSLLLFLLLQHWRQGKHR |
| | | TLAQRQADFQRPPGAAEPEPKDGGLQRRSSPAADVQGENFCAAVKNTQPEDGV |
| | | EMDTRQSPHDEDPQAVTYAKVKHSRPRREMASPPSPLSGEFLDTKDRQAEEDR |
| | | QMDTEAAASEAPQDVTYAQLHSFTLRQKATEPPPSQEGASPAEPSVYA |
| Human | 5 | GTIPKPTLWAEPDSVITQGSPVTLSCQGSLEAQEYRLYREKKSASWITRIRPE |
| ILT-4 | | LVKNGQFHIPSITWEHTGRYGCQYYSRARWSELSDPLVLVMTGAYPKPTLSAQ |
| | | PSPVVTSGGRVTLQCESQVAFGGFILCKEGEEEHPQCLNSQPHARGSSRAIFS |
| | | VGPVSPNRRWSHRCYGYDLNSPYVWSSPSDLLELLVPGVSKKPSLSVQPGPVV |
| | | APGESLTLQCVSDVGYDRFVLYKEGERDLRQLPGRQPQAGLSQANFTLGPVSR |
| | | SYGGQYRCYGAHNLSSECSAPSDPLDILITGQIRGTPFISVQPGPTVASGENV |
| | | TLLCQSWRQFHTFLLTKAGAADAPLRLRSIHEYPKYQAEFPMSPVTSAHAGTY |
| | | RCYGSLNSDPYLLSHPSEPLELVVSGPSMGSSPPPTGPISTPGPEDQPLTPTG |
| | | SDPQSGLGRHLGVVIGILVAVVLLLLLLLLLFLILRHRRQGKHWTSTQRKADF |
| | | QHPAGAVGPEPTDRGLQWRSSPAADAQEENLYAAVKDTQPEDGVEMDTRAAAS |
| | | EAPQDVTYAQLHSLTLRRKATEPPPSQEREPPAEPSIYATLAIH |
| Human | 6 | GPFPKPTLWAEPGSVISWGSPVTIWCQGSLEAQEYRLDKEGSPEPLDRNNPLE |
| ILT-5 | | PKNKARFSIPSMTEHHAGRYRCHYYSSAGWSEPSDPLELVMTGFYNKPTLSAL |
| | | PSPVVASGGNMTLRCGSQKGYHHFVLMKEGEHQLPRTLDSQQLHSGGFQALFP |
| | | VGPVNPSHRWRFTCYYYYMNTPQVWSHPSDPLEILPSGVSRKPSLLTLQGPVL |
| | | APGQSLTLQCGSDVGYDRFVLYKEGERDFLQRPGQQPQAGLSQANFTLGPVSP |
| | | SHGGQYRCYGAHNLSSEWSAPSDPLNILMAGQIYDTVSLSAQPGPTVASGENV |
| | | TLLCQSWWQFDTFLLTKEGAAHPPLRLRSMYGAHKYQAEFPMSPVTSAHAGTY |
| | | RCYGSYSSNPHLLSHPSEPLELVVSGHSGGSSLPPTGPPSTPGLGRYLEVLIG |
| | | VSVAFVLLLFLLLFLLLRRQRHSKHRTSDQRKTDFQRPAGAAETEPKDRGLLR |
| | | RSSPAADVQEENLYAAVKDTQSEDRVELDSQSPHDEDPQAVTYAPVKHSSPRR |
| | | EMASPPSSLSGEFLDTKDRQVEEDRQMDTEAAASEASQDVTYAQLHSLTLRRK |
| | | ATEPPPSQEGEPPAEPSIYA |
| Human | 7 | GPLPKPTLWAEPGSVITQGSPVTLRCQGSLETQEYHLYREKKTALWITRIPQE |
| ILT-6 | | LVKKGQFPILSITWEHAGRYCCIYGSHTAGLSESSDPLELVVTGAYSKPTLSA |
| | | LPSPVVTSGGNVTIQCDSQVAFDGFILCKEGEDEHPQCLNSHSHARGSSRAIF |
| | | SVGPVSPSRRWSYRCYGYDSRAPYVWSLPSDLLGLLVPGVSKKPSLSVQPGPV |
| | | VAPGEKLTFQCGSDAGYDRFVLYKEWGRDFLQRPGRQPQAGLSQANFTLGPVS |
| | | RSYGGQYTCSGAYNLSSEWSAPSDPLDILITGQIRARPFLSVRPGPTVASGEN |
| | | VTLLCQSQGGMHTFLLTKEGAADSPLRLKSKRQSHKYQAEFPMSPVTSAHAGT |
| | | YRCYGSLSSNPYLLTHPSDPLELVVSGAAETLSPPQNKSD |
| Human | 8 | ENLPKPILWAEPGPVITWHNPVTIWCQGTLEAQGYRLDKEGNSMSRHILKTLE |
| ILT-7 | | SENKVKLSIPSMMWEHAGRYHCYYQSPAGWSEPSDPLELVVTAYSRPTLSALP |
| | | SPVVTSGVNVTLRCASRLGLGRFTLIEEGDHRLSWTLNSHQHNHGKFQALFPM |
| | | GPLTFSNRGTFRCYGYENNTPYVWSEPSDPLQLLVSGVSRKPSLLTLQGPVVT |
| | | PGENLTLQCGSDVGYIRYTLYKEGADGLPQRPGRQPQAGLSQANFTLSPVSRS |
| | | YGGQYRCYGAHNVSSEWSAPSDPLDILIAGQISDRPSLSVQPGPTVTSGEKVT |
| | | LLCQSWDPMFTFLLTKEGAAHPPLRLRSMYGAHKYQAEFPMSPVTSAHAGTYR |
| | | CYGSRSSNPYLLSHPSEPLELVVSGATETLNPAQKKSDSKTAPHLQDYTVENL |
| | | IRMGVAGLVLLFLGILLFEAQHSQRSPPRCSQEANSRKDNAPFRVVE |
| Human | 9 | GPFPKPTLWAEPGSVISWGSPVTIWCQGSLEAQEYQLDKEGSPEPLDRNNPLE |
| ILT-8 | | PKNKARFSIPSMTQHHAGRYRCHYYSSAGWSEPSDPLELVMTGFYNKPTLSAL |
| | | PSPVVASGGNMTLRCGSQKGYHHFVLMKEGEHQLPRTLDSQQLHSGGFQALFP |
| | | VGPVTPSHRWRFTCYYYYTNTPRVWSHPSDPLEILPSGVSRKPSLLTLQGPVL |
| | | APGQSLTLQCGSDVGYDRFVLYKEGERDFLQRPGQQPQAGLSQANFTLGPVSP |
| | | SHGGQYRCYGAHNLSSEWSAPSDPLNILMAGQIYDTVSLSAQPGPTVASGENV |
| | | TLLCQSRGYFDTFLLTKEGAAHPPLRLRSMYGAHKYQAEFPMSPVTSAHAGTY |
| | | RCYGSYSSNPHLLSFPSEPLELMVSASHAKDYTVENLIRMGMAGLVLVFLGIL |
| | | LFEAQHSQRNPQD |

Briefly, for the flow cytometry screening, antibodies were incubated 1 hour with each ILT-expressing CHO cell lines (CHO ILT1 cell line, CHO ILT2 cell line, CHO ILT3 cell line, CHO ILT4 cell line, CHO ILT5 cell line, CHO ILT6 cell line, CHO ILT7 cell line, CHO ILT8 cell line), washed twice in staining buffer, revealed by Goat anti-mouse IgG H+L polyclonal antibody (pAb) labeled with PE (for commercially available antibodies, Jackson Immuoresearch #115-116-146) or Goat anti-human IgG H+L pAb labeled with PE (for chimeric antibodies, Jackson Immunoresearch #109-116-088) washed twice with staining buffer and stainings were acquired on a Accury C6 flowcytometer equipped with an HTFC plate loader and analyzed using the FlowJo software.

Results showed that many of the anti-ILT2 antibodies bound also to ILT6 (LILRA3) in addition to ILT2, either alone (i.e. ILT2/ILT6 cross-reactive) or with additional binding to ILT4 or ILT5 (i.e. ILT2/ILT4/ILT6 or ILT2/ILT5/ILT6 cross-reactive). Antibodies 1C11, 1D6, 9G1, 19F10a, 27G10, commercial antibodies 586326 and 292305 bound to ILT2 and also ILT6. Antibody 586326 furthermore also bound to ILT4 in addition to ILT2 and ILT6, whereas antibody 292305 further bound ILT5 in addition to ILT2 and ILT6. Finally, commercial antibody 292319 bound to ILT1 in addition to ILT2 (ILT1/ILT2 cross-reactive). However, a subset of antibodies exemplified by 3H5, 12D12, 26D8, 18E1, 27C10 and 27H5 bound only to ILT2 and no other ILT family member protein.

### Example 11: Epitope Mapping

### Anchored ILT2 Domain Fragment Proteins

### Generation of ILT2 Proteins

Nucleic acid sequences encoding different human ILT2 domains D1 (corresponding to residues 24-121 of the sequence shown in SEQ ID NO: 1), D2 (corresponding to residues 122-222 of the sequence shown in SEQ ID NO: 1), D3 (corresponding to residues 223-321 of the sequence shown in SEQ ID NO: 1), D4 (corresponding to residues 322-458 of the sequence shown in SEQ ID NO: 1), and combinations thereof, were amplified by PCR using the primers described in the Table below. The PCR products were inserted into an expression vector at appropriate restriction sites. A heavy chain peptide leader was used and a V5 tag was added at the N-terminal and expression at the surface of cells was confirmed by flow cytometry. For all of the domains that were not followed by a D4 domain, a CD24 GPI anchor was added to permit anchoring at the cell membrane. The amino acid sequences of the resulting different human ILT2 domain fragment-containing proteins are shown below in Table 5, below. The vectors were then transfected into the CHO cell line to obtain stable clones expressing the different ILT2 domain proteins at the cell surface.

TABLE 5

| | | SEQ ID |
|---|---|---|
| Description | Amino acid sequence | NO |
| D1 domain | TGVHSGKPIPNPLLGLDSTGHLPKPTLWAEPGSVITQGSPVTLRCQGGQETQ | 46 |
| | EYRLYREKKTALWITRIPQELVKKGQFPIPSITWEHAGRYRCYYGSDTAGRS | |
| | ESSDPLELVVTGAGALQSTASLFVVSLSLLHLYS | |
| D2 domain | TGVHSGKPIPNPLLGLDSTYIKPTLSAQPSPVVNSGGNVILQCDSQVAFDGF | 47 |
| | SLCKEGEDEHPQCLNSQPHARGSSRAIFSVGPVSPSRRWWYRCYAYDSNSPY | |
| | EWSLPSDLLELLVLGVGALQSTASLFVVSLSLLHLYS | |
| D3 domain | TGVHSGKPIPNPLLGLDSTSKKPSLSVQPGPIVAPEETLTLQCGSDAGYNRF | 48 |
| | VLYKDGERDFLQLAGAQPQAGLSQANFTLGPVSRSYGGQYRCYGAHNLSSEW | |
| | SAPSDPLDILIAGQGALQSTASLFVVSLSLLHLYS | |
| D4 domain | TGVHSGKPIPNPLLGLDSTFYDRVSLSVQPGPTVASGENVTLLCQSQGWMQT | 49 |
| | FLLTKEGAADDPWRLRSTYQSQKYQAEFPMGPVTSAHAGTYRCYGSQSSKPY | |
| | LLTHPSDPLELVVSGPSGGPSSPTTGPTSTSGPEDQPLTPTGSDPQSGLGRH | |
| | LGVVIGILVAVILLLLLLLLLFLILRHRRQGKHWTSTQRKADFQHPAGAVGP | |
| | EPTDRGLQWRSSPAADAQEENLYAAVKHTQPEDGVEMDTRSPHDEDPQAVTY | |
| | AEVKHSRPRREMASPPSPLSGEFLDTKDRQAEEDRQMDTEAAASEAPQDVTY | |
| | AQLHSLTLRREATEPPPSQEGPSPAVPSIYATLAIH | |
| D1-D2 | TGVHSGKPIPNPLLGLDSTGHLPKPTLWAEPGSVITQGSPVTLRCQGGQETQ | 50 |
| domain | EYRLYREKKTALWITRIPQELVKKGQFPIPSITWEHAGRYRCYYGSDTAGRS | |
| | ESSDPLELVVTGAYIKPTLSAQPSPVVNSGGNVILQCDSQVAFDGFSLCKEG | |
| | EDEHPQCLNSQPHARGSSRAIFSVGPVSPSRRWWYRCYAYDSNSPYEWSLPS | |
| | DLLELLVLGVGALQSTASLFVVSLSLLHLYS | |
| D2-D3 | TGVHSGKPIPNPLLGLDSTYIKPTLSAQPSPVVNSGGNVILQCDSQVAFDGF | 51 |
| domain | SLCKEGEDEHPQCLNSQPHARGSSRAIFSVGPVSPSRRWWYRCYAYDSNSPY | |
| | EWSLPSDLLELLVLGVSKKPSLSVQPGPIVAPEETLTLQCGSDAGYNRFVLY | |
| | KDGERDFLQLAGAQPQAGLSQANFTLGPVSRSYGGQYRCYGAHNLSSEWSAP | |
| | SDPLDILIAGQGALQSTASLFVVSLSLLHLYS | |
| D3-D4 | TGVHSGKPIPNPLLGLDSTSKKPSLSVQPGPIVAPEETLTLQCGSDAGYNRF | 52 |
| domain | VLYKDGERDFLQLAGAQPQAGLSQANFTLGPVSRSYGGQYRCYGAHNLSSEW | |
| | SAPSDPLDILIAGQFYDRVSLSVQPGPTVASGENVTLLCQSQGWMQTFLLTK | |
| | EGAADDPWRLRSTYQSQKYQAEFPMGPVTSAHAGTYRCYGSQSSKPYLLTHP | |
| | SDPLELVVSGPSGGPSSPTTGPTSTSGPEDQPLTPTGSDPQSGLGRHLGVVI | |
| | GILVAVILLLLLLLLLFLILRHRRQGKHWTSTQRKADFQHPAGAVGPEPTDR | |
| | GLQWRSSPAADAQEENLYAAVKHTQPEDGVEMDTRSPHDEDPQAVTYAEVKH | |
| | SRPRREMASPPSPLSGEFLDTKDRQAEEDRQMDTEAAASEAPQDVTYAQLHS | |
| | LTLRREATEPPPSQEGPSPAVPSIYATLAIH | |
| D1-D2-D3 | TGVHSGKPIPNPLLGLDSTGHLPKPTLWAEPGSVITQGSPVTLRCQGGQETQ | 53 |
| domain | EYRLYREKKTALWITRIPQELVKKGQFPIPSITWEHAGRYRCYYGSDTAGRS | |
| | ESSDPLELVVTGAYIKPTLSAQPSPVVNSGGNVILQCDSQVAFDGFSLCKEG | |
| | EDEHPQCLNSQPHARGSSRAIFSVGPVSPSRRWWYRCYAYDSNSPYEWSLPS | |
| | DLLELLVLGVSKKPSLSVQPGPIVAPEETLTLQCGSDAGYNRFVLYKDGERD | |
| | FLQLAGAQPQAGLSQANFTLGPVSRSYGGQYRCYGAHNLSSEWSAPSDPLDI | |
| | LIAGQGALQSTASLFVVSLSLLHLYS | |
| D2-D3-D4 | TGVHSGKPIPNPLLGLDSTYIKPTLSAQPSPVVNSGGNVILQCDSQVAFDGF | 54 |
| domain | SLCKEGEDEHPQCLNSQPHARGSSRAIFSVGPVSPSRRWWYRCYAYDSNSPY | |
| | EWSLPSDLLELLVLGVSKKPSLSVQPGPIVAPEETLTLQCGSDAGYNRFVLY | |
| | KDGERDFLQLAGAQPQAGLSQANFTLGPVSRSYGGQYRCYGAHNLSSEWSAP | |
| | SDPLDILIAGQFYDRVSLSVQPGPTVASGENVTLLCQSQGWMQTFLLTKEGA | |
| | ADDPWRLRSTYQSQKYQAEFPMGPVTSAHAGTYRCYGSQSSKPYLLTHPSDP | |
| | LELVVSGPSGGPSSPTTGPTSTSGPEDQPLTPTGSDPQSGLGRHLGVVIGIL | |
| | VAVILLLLLLLLLFLILRHRRQGKHWTSTQRKADFQHPAGAVGPEPTDRGLQ | |
| | WRSSPAADAQEENLYAAVKHTQPEDGVEMDTRSPHDEDPQAVTYAEVKHSRP | |
| | RREMASPPSPLSGEFLDTKDRQAEEDRQMDTEAAASEAPQDVTYAQLHSLTL | |
| | RREATEPPPSQEGPSPAVPSIYATLAIH | |

Results

The ILT2 selective antibodies were tested for their binding to the different anchored ILT2 fragments by flow cytometry. 3H5, 12D12 and 27H5 all bound to the D1 domain of ILT2. These antibodies bound to all cells that expressed proteins that contained the D1 domain of ILT2, (the proteins of SEQ ID NOS: 46, 50 and 53) without binding to any of the cells that expressed the ILT2 proteins that lacked the D1 domain (the proteins of SEQ ID NOS: 47-49, 51, 52 and 54). The antibodies 3H5, 12D12 and 27H5 thus bind to a domain of ILT2 defined by residues 24-121 of the sequence shown in SEQ ID NO: 1 (also referred to as domain D1). Antibodies 26D8, 18E1 and 27C10 all bound to the D4 domain of ILT2. These antibodies bound to all cells that expressed proteins that contained the D4 domain of ILT2, (the proteins of SEQ ID NOS: 49, 52 and 54) without binding to any of the cells that expressed the ILT2 proteins that lacked the D4 domain (the proteins of SEQ ID NOS: 46-28, 50, 51, or 53). The antibodies 26D8, 18E1 and 27C10 thus bind to a domain of ILT2 defined by residues 322-458 of the sequence shown in SEQ ID NO: 1. FIG. 7 shows a representative example binding of the antibodies to the anchored ILT2 domain D1 fragment protein of SEQ ID NO: 46 (left hand panel), the D3 domain fragment protein of SEQ ID NO: 48 (middle panel), and the D4 domain protein of SEQ ID NO: 49 (right hand panel).

### ILT2 Point Mutation Study

The identification of antibodies that bound ILT2 without binding to the closely related ILT6 permitted the design of ILT2 mutations on amino acids exposed and different between ILT2 and ILT6. Anti-ILT2 antibodies that did not cross-react on ILT6 could then be mapped for loss of binding to different ILT2 mutants having amino acid substitutions in the D1, D2 or D4 domains of ILT2. The loss of binding to an ILT2 mutant together with loss of binding to human ILT6 can serve to identify to epitope on ILT2 bound by the antibodies that enhance NK cell cytotoxicity.

### Generation of ILT2 Mutants

ILT2 mutants were generated by PCR. The sequences amplified were run on agarose gel and purified using the Macherey Nagel PCR Clean-Up Gel Extraction kit (reference 740609). The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO2 incubator for 48 hours prior to testing for transgene expression. Mutants were transfected in Hek-293T cells, as shown in the table below. The targeted amino acid mutations are shown in the Table 6 below, listing the residue present in wild-type ILT2/position of residue/residue present in mutant ILT2, with position reference being to either the ILT2 protein lacking leader peptide shown in SEQ ID NO: 2 in the left column, or to the ILT2 protein with leader peptide shown in SEQ ID NO: 1 in the right column.

TABLE 6

| | Amino acid substitutions with | Amino acid substitutions with reference |
|---|---|---|
| | reference to ILT2 lacking leader | to ILT2 having leader peptide of |
| Mutant | peptide of SEQ ID NO: 2 | SEQ ID NO: 1 |
| 1 | G295 - Q3OL - T32A - Q33A - D80H | G525 - Q53L - T55A - Q56A - D103H |
| 2 | E34A - R36A - Y76I - A82S - R84L | E57A - R59A - Y99I - A105S - R107L |
| 3 | Y99A - I100S - V126S - A127S - | Y122A - I123S - V149S - A150S - |
| | D129A - N180R - S181A - E184G | D152A - N203R - 5204A - E207G |
| 3b | Q18A - W67A - Y99A - I100S - | Q41A - W90A - Y122A - I123S- |
| | V126S - S181A - E184G | V149S - S204A - E207G |
| 4 | S132A - L145S - N146A - Q148H - | S155A - L168S - N169A - Q171H - |
| | P149S | P172S |
| 5 | A1275 - D129A - Q148H - R152A - | A1505 - D152A - Q171H - R175A - |
| | N180R | N203R |
| 6 | Q107L - P108A - I119A - R156A | Q130L - P131A - I142A - R179A |
| 7 | P166A - R169A - W171S - L191A - | P189A - R192A - W194S - L214A - |
| | E193G - L1955 - L197P | E216G - L2185 - L220P |
| 8 | V111S - N113A - L195S - L197P | V134S - N136A - L218S - L220P |
| 4-1 | F2991 - Y300R - D301A - W328G - | F3221 - Y323R - D324A - W351G - |
| | Q378A - K381N | Q401A - K404N |
| 4-1b | Y300R - D301A - R302A - 5304F - | Y323R - D324A - R325A - S327F - |
| | H387A - D390A | H410A - D413A |
| 4-2 | W328G - Q330H - R347A - T349A - | W351G - Q353H - R370A - T372A - |
| | Y350S - Y355A | Y373S - Y378A |
| 4-3 | Q324A - Q3265 - S352A - Q353H - | Q347A - Q3495 - S375A - Q376H - |
| | K354A | K377A |
| 4-4 | Q308A - P309G - N318A - T320A - | Q331A - P332G - N341A - T343A - |
| | E3585 - G3625 | E3815 - G3855 |
| 4-5 | D341A - D3425 - W344L - R345A - | D364A - D3655 - W367L - R368A - |
| | R347A | R370A |

### Results

The ILT2 selective antibodies were tested for their binding to each of mutants by flow cytometry. A first experiment was performed to determine antibodies that lose their binding to one or several mutants at one concentration. To confirm a loss of binding, titration of antibodies was done on antibodies for which binding seemed to be affected by the ILT2 mutations. A loss or decrease of binding for a test antibody indicated that one or more, or all of, the residues of the particular mutant are important to the core epitope of the antibodies, and thereby permitted the region of binding of ILT2 to be identified.

Antibodies 3H5, 12D12 and 27H5 bound an epitope in domain D1 of ILT2, as these three antibodies lost binding to mutant 2 having amino acid substitutions at residues 34, 36, 76, 82 and 84 (substitutions E34A, R36A, Y76I, A82S, R84L) in the domain 1 (D1 domain) of ILT2. 12D12 and 27H5 did not lose binding to any other mutant, however 3H5 also had a decrease (partial loss) of binding to mutant 1 having amino acid substitutions at residues 29, 30, 33, 32, 80 (substitutions G29S, Q30L, Q33A, T32A, D80H). These amino acid residues, together with lack of binding to human ILT6 polypeptide, therefore can identify an epitope that characterizes anti-ILT2 antibodies that enhance cytotoxicity in primary NK cells.

FIG. 8A shows a representative example of titration of antibodies 3H5, 12D12 and 27H5 for binding to mutants 1 and 2 by flow cytometry. FIG. 9A shows a model representing a portion of the ILT2 molecule that includes domain 1 (top portion, shaded in dark gray) and domain 2 (bottom, shaded in light gray). The figure shows the binding site of the antibodies as defined by the amino acid residues substituted in mutant 1 (M1) and mutant 2 (M2).

Antibodies 26D8, 18E1 and 27C10 all bound an epitope in domain D4 of ILT2. Antibodies 26D8 and 18E1 lost binding to mutants 4-1 and 4-2. Mutant 4-1 has amino acid substitutions at residues 299, 300, 301, 328, 378 and 381 (substitutions F299I, Y300R, D301A, W328G, Q378A, K381N). Mutant 4-2 has amino acid substitutions at residues 328, 330, 347, 349, 350 and 355 (substitutions W328G, Q330H, R347A, T349A, Y350S, Y355A). 26D8 furthermore lost binding to mutant 4-5, while antibody 18E1 had a decrease in binding (but not complete loss of binding) to mutant 4-5. 27C10 also lost binding to mutant 4-5, but not to any other mutant. Mutant 4-5 has amino acid substitutions at residues 341, 342, 344, 345 and 347 (substitutions D341A, D342S, W344L, R345A, R347A). 26D8 and 18E1 did not lose binding to any other mutants. These amino acid residues, together with lack of binding to human ILT6 polypeptide, therefore can identify an epitope that characterizes anti-ILT2 antibodies that enhance cytotoxicity in primary NK cells.

FIG. 8B shows a representative example of titration of antibodies 26D8, 18E1 and 27C10 for binding to D4 domain mutants 4-1, 4-1b, 4-2, 4-4 and 4-5 by flow cytometry

FIG. 9B shows a model representing a portion of the ILT2 molecule that includes domain 3 (top portion, shaded in dark gray) and domain 4 (bottom, shaded in light gray). The figure shows the binding site of the antibodies as defined by the amino acid residues substituted in mutants, 4-1, 4-2 and 4-5 which are all located within domain 4 of ILT2. Antibodies 26D8, 18E1 which potentiate the cytotoxicity of primary NK cells bind the site defined by mutants 4-1 and 4-2 without binding to the site defined by mutant 4-5, while antibodies 27C10 which did not potentiate the cytotoxicity of primary NK cells binds to the site defined by mutant 4-5.

### Example 12: Affinity Binding Threshold for Enhancement of Cytotoxicity in Primary Human NK Cells by ILT2-HLA-G Blocking Antibodies

In order to better understand the mechanism underlying the activity of the anti-ILT2 antibodies that were highly active in enhancing primary NK cell cytotoxicity, a further immunization and screening was carried out using the methods described in Example 3, combined with additional screening for binding to closely related ILT family members as in Example 10.

Balb/c mice were immunized with ILT-2_6×His protein. After the immunization protocol the mice were sacrificed to perform fusions and get hybridomas. The hybridoma supernatants were used to stain ILT-expressing CHO-cell lines described in Example 10 (CHO lines each expressing one of ILT1 (LILRA2), ILT3 (LILRB4), ILT4 (LILRB2), ILT5 (LILRB3), ILT6 (LILRA3) or ILT7 (LILRA4) to check for monoclonal antibody reactivities in a flow cytometry experiment. Briefly, the cells were incubated with 50 μl of supernatant for 1H at 4° C., washed three times and a secondary antibody Goat anti-mouse IgG Fc specific antibody coupled to AF647 was used (Jackson Immunoresearch, JI115-606-071). After 30 min of staining, the cells were washed three times and analyzed using a FACS CANTO II (Becton Dickinson).

Antibodies were cloned and screened, to identify those producing antibodies that bind to ILT2 without binding to human ILT1, ILT3, ILT4, ILT5, ILT6, or ILT7 and which have the ability to block the interaction between ILT2 with HLA-G. Briefly, recombinant biotinylated ILT2 was incubated with APC-conjugated streptavidin for 20 min at 4° C. prior addition of purified anti-ILT2 antibodies. After 20 min, the complexes were incubated with 5×10⁴ K562 cells expressing HLA-G or WIL2-NS cells expressing HLA-A2 for 30 supplemental min at 4° C. Cells were washed once in PBS and fixed with Cell Fix (Becton Dickinson, 340181). Analysis was performed on a FACS CANTO II flow cytometer.

Ability of anti-ILT2 antibodies to block the interactions between HLA-G or HLA-A2 expressed at the surface of cell lines and recombinant ILT2 protein was assessed by flow cytometry, as described in Example 5. These assays permitted the identification of a panel of anti-ILT2 antibodies that were highly effective in blocking the interaction of ILT2 with its HLA class I ligand HLA-G. Antibodies 12D12, 2A8A, 2A8B, 2A9, 2611, 2C4, 2C8, 2D8, 2E2B, 2E2C, 2E8, 2E11, 2G5, 2H2A, 2H2B, 2H12, 1A9, 1A10B, 1A10C, 1A10D, 1E4B, 1E4C, 3A7A, 3A7B, 3A8, 3B5, 3E5, 3E7A, 3E7B, 3E9A, 3E9B, 3F5, 4A8, 4C11B, 4E3A, 4E3B, 4H3, 5C5, 5D9, 6C6, 10H1, 48F12, 15D7, 2C3 all blocked ILT2 binding to HLA-G and HLA-A2. FIG. 10A shows representative results for antibodies 12D12, 2H2B, 48F12, 1E4C, 1A9, 3F5 and 3A7A. The resulting antibodies were tested for their binding to the different anchored ILT2 fragments and ILT2 point mutants by flow cytometry as shown in Example 11, and produced as modified chimeric antibodies having human IgG1 Fc domains with the mutations L234A/L235E/G237A/A330S/P331S.

Ability of anti-ILT2 antibodies to increase cytotoxicity in primary human NK cells was tested as in Example 9. Briefly, the effect of the anti-ILT2 antibodies on NK cells activation was determined by flow cytometry of CD137 expression on total NK cells, ILT2-positive NK cells and ILT2-negative NK cells. Effector cells were primary NK cells (fresh NK cells purified from donors, incubation overnight at 37° C. before use) and target cells (WIL2-NS cell line) were mixed at a ratio 1:1.

FIG. 10B is a representative figure showing the increase of % of total NK cells expressing CD137 mediated by anti-ILT2 antibodies 12D12, 2H2B, 48F12, 1E4C, 1A9, 3F5 and 3A7A using NK cells from two human donors and WIL2-NS that endogenously express HLA-A2. Antibodies showed strong activation of the primary NK cells. Study of ILT2-positive NK cells showed that these antibodies mediated a strong increase in activation of the NK cells toward the target cells. The characterization of their epitope on the point mutants showed that similarly to antibodies 3H5, 12D12 and 27H5, the antibodies 2H2B, 48F12 and 3F5 that were tested for domain binding all bound to the D1 domain of ILT2; they bound to all cells that expressed proteins that contained the D1 domain of ILT2, (the proteins of SEQ ID NOS: 46, 50 and 53) without binding to any of the cells that expressed the ILT2 proteins that lacked the D1 domain (the proteins of SEQ ID NOS: 47-49, 51, 52 and 54). When tested for binding to ILT-2 point mutants, Antibodies 12D12, 2H2B, 48F12, 1E4C, 1A9, 3F5 and 3A7A bound an epitope in domain D1 of ILT2, with loss of binding to mutant 2 having amino acid substitutions at residues 34, 36, 76, 82 and 84 (substitutions E34A, R36A, Y76I, A82S, R84L) in the domain 1 (D1 domain) of ILT2.

These results led to the observation that surprisingly some antibodies that were effective in blocking the interactions between HLA-G or HLA-A2 expressed at the surface of cell and bound the same area on the D1 domain of ILT2 were not necessarily able to mediate a an increase in or restore cytotoxicity of the primary human NK cells. In particular, as shown in FIG. 10B, antibodies 1E4C, 1A9 and 3A7A, despite being from the same murine V gene combinations as other antibodies (1E4C, 1A9 and 3A7A were from IGHV1-66*01 or IGHV1-84*01 genes combined with IGKV3-5*01), substantially lacked the ability to activate the primary NK cells all, compared to isotype control antibodies. Epitope mapping showed that these antibodies indeed bound to the D1 domain of ILT2; they bound to all cells that expressed proteins that contained the D1 domain of ILT2, (the proteins of SEQ ID NOS: 46, 50 and 53) without binding to any of the cells that expressed the ILT2 proteins that lacked the D1 domain (the proteins of SEQ ID NOS: 47-49, 51, 52 and 54), and that they showed loss of binding to mutant 2 having amino acid substitutions at residues 34, 36, 76, 82 and 84 (substitutions E34A, R36A, Y76I, A82S, R84L) in the domain 1 (D1 domain) of ILT2.

As part of an investigation into why these anti-D1 epitope antibodies did not function to enhance NK cell cytotoxicity in primary NK cells, we observed that for several antibodies that activated primary NK cells, there were also other antibodies having closely related variable region sequences which did not activate primary NK cells (despite being potent ILT2-HLA-G blockers. It may therefore be that the differences (in CDR residues in particular) may affect the affinity of the antibodies. The antibodies with CDRs derived from the same variable region genes were grouped and further characterized for their monovalent binding affinity to human ILT2 using the methods of Example 8. Briefly, anti-ILT2 antibodies at 1 μg/mL were captured onto a Protein-A chip and recombinant human ILT2 proteins were injected at 5 μg/mL over captured antibodies. For blank subtraction, cycles were performed again replacing ILT2 proteins with running buffer. The monovalent affinity analysis was conducted following a regular Capture-Kinetic protocol as recommended by the manufacturer (Biacore GE Healthcare kinetic wizard). Results are shown in Table 5, below. The antibodies 1E4C, 1A9 and 3A7A that blocked HLA-G and HLA-A2 but that did not enhance cytotoxicity of the primary human NK cells engaged the ILT-2 protein rapidly (ka in Table 5), however were characterized by a fast dissociation compared to the antibodies that are able to enhance cytotoxicity of the primary human NK cells. In particular, 1E4C, 1A9 and 3A7A were characterized by a 2 state reaction, in which the antibodies dissociate in two phases, a first rapid “kd1” phase and a second slower “kd2” phase. The first phase for 1E4C, 1A9 and 3A7A was characterized by a kd of greater than 1E-2. It therefore appears that while strong affinity in binding (on rate) may suffice to block the ILT2-HLA-G/A2 interaction in in vitro assays, a lower dissociation rate is required to enhance NK cell cytotoxicity. Differences in KD between the different D1 domain epitope antibodies was also generally observed, although less important than the kd. Results show that despite the ability of the anti-D1 domain epitope antibodies to potently block the interaction of ILT-2 with its HLA ligands, there is a threshold of affinity that is required to enhance NK cell cytotoxicity in primary NK cells.

Antibodies 2A8A, 2A9, 2C4, 2C8, 2D8, 2E2B, 2E2C, 2E8, 2E11, 2H2A, 2H12, 1A10D, 3E5, 3E7A, 3E7B, 3E9B, 3F5, 4C11B, 4E3A, 4E3B, 4H3, 5D9 and 6C6 had heavy chain variable region/CDRs derived from the murine IGHV1-66*01 gene and light chain variable region/CDRs derived from the murine IGKV3-5*01 gene. 1E4B had heavy chain variable region/CDRs derived from the murine IGHV1-66*01 gene and light chain variable region/CDRs derived from the murine IGKV3-4*01. 2H2B had heavy chain variable region/CDRs derived from the murine IGHV1-84*01 gene and light chain variable region/CDRs derived from the murine IGKV3-5*01 gene. The antibodies that activated primary NK cells displayed variable residues present at various positions in their VH and HCDRs as Kabat positions 32-35, 52A, 54, 55, 56, 57, 58, 60, 65, 95 and 101, and variable residues present at various positions in their VL and LCDRs as Kabat positions 24, 25, 26, 27, 27A, 28, 33, 34, 50, 53, 55, 91, 94 and 96.

48F12 had heavy chain variable region/CDRs derived from the murine IGHV2-3*01 gene and light chain variable region/CDRs derived from the murine IGKV10-96*02 gene.

The NK cell cytotoxicity-enhancing anti-D1 epitope antibodies 12D12, 2A8A, 2A9, 2C4, 2C8, 2D8, 2E2B, 2E2C, 2E8, 2E11, 2H2A, 2H2B, 2H12, 1A10D, 1E4B, 3E5, 3E7A, 3E7B, 3E9B, 3F5, 4C11B, 4E3A, 4E3B, 4H3, 5D9, 6C6 or 48F12 were characterized by a loss of binding to cells expressing ILT2 mutant 2 having amino acid substitutions at residues 34, 36, 76, 82 and 84 (substitutions E34A, R36A, Y761, A82S, R84L), loss of binding to the human ILT-6 polypeptide, along with 1:1 Binding fit and/or dissociation or off rate (kd (1/s)) of less than 1E-2 or 1E-3 (monovalent binding affinity assay, as determined by SPR).

TABLE 5

| mAb | Fit | KD (nM) | ka (1/Ms) | kd (1/s) |
|---|---|---|---|---|
| 1A9 | Two State | 7.5 | ka1: | kd1: |
| | Reaction | | 3.4E+5 | 3.5E−2 |
| | | | ka2: | kd2: |
| | | | 2.7E−3 | 2.1E−4 |
| 1E4C | Two State | 1.8 | ka1: | kd1: |
| | Reaction | | 1.1E+6 | 3.0E−2 |
| | | | ka2: | kd2: |
| | | | 1.9E−3 | 1.3E−4 |
| 3A7A | Two State | 3.7 | ka1: | kd1: |
| | Reaction | | 8.6E+5 | 3.1E−2 |
| | | | ka2: | kd2: |
| | | | 1.8E−3 | 2.1E−4 |
| 2H2B | 1:1 Binding | 0.8 | 1.4E+6 | 1.1E−3 |
| 48F12 | 1:1 Binding | 0.2 | 5.0E+5 | 1.0E−4 |
| 3F5 | 1:1 Binding | 1.9 | 1.2E+6 | 2.2E−3 |

### Example 13: Antibodies Enhance NK Cell-Mediated ADDC

### Anti-ILT2 Antibodies Enhance NK Cell Cytotoxicity of Rituximab Towards Tumor Cells

The effect of the anti-ILT2 antibodies on NK cell activation was determined by analysis by flow cytometry of CD137 expression on NK cells, ILT2-positive NK cells and ILT2-negative NK cells from human tumor cells.

Tumor target cells were WIL2-NS tumor target cells in which ILT-2 was silenced. Effector cells (fresh NK cells purified from human healthy donors) and tumor target cells were mixed at a ratio 1:1. The CD137 assay was carried out in 96 U well plates in completed RPMI, 200 μL final/well. Antibodies used included anti-ILT-2 antibodies 12D12, 18E1 and 26D8 at a concentration of 10 μg/mL, isotype control antibodies, in combination with rituximab at a concentration of 0.001 μg/mL. Antibodies were pre-incubated 30 minutes at 37° C. with effector cells and then target cells were co-incubated overnight at 37° C. The following steps were: spin 3 min at 400 g; wash twice with Staining Buffer (SB); addition of 50 μL of staining Ab mix (anti-CD3 Pacific blue-BD Biosciences; anti-CD56-PE-Vio770-Miltenyi Biotec; anti-CD137-APC-Miltenyi Biotec; anti-ILT2-PE—clone HP—F1, eBioscience); incubation 30 min at 4° C.; wash twice with SB; resuspended pellet with Cellfix-Becton Dickinson; and fluorescence revealed with a FACS Canto II flow cytometer (Becton Dickinson). Negative controls were NK cells vs target cells alone and in presence of isotype control.

The anti-ILT2 antibodies were able to mediate a strong increase of NK cell cytotoxicity mediated by rituximab. Surprisingly, the combination of anti-ILT2 antibodies and rituximab resulted in stronger activation of total NK cell activation than either agent was able to mediate on its own. FIG. 11A shows the fold increase over rituximab alone (compared to medium) in activation of NK cells following incubation with rituximab without or without anti-ILT2 antibodies, and the tumor target cells, in five human donors. Each of the anti-ILT2 antibodies 12D12, 18E1 and 26D8 resulted in an increase of the NK cytotoxicity mediated by rituximab alone. The combination increased NK cell cytotoxicity of rituximab in the LILRB1+ population of NK cells and in the entire NK cell population.

### Anti-ILT2 Antibodies Enhance NK Cell Cytotoxicity of Cetuximab Towards Tumor Cells

The effect of the anti-ILT2 antibodies on NK cell activation was determined by analysis by flow cytometry of CD137 expression on NK cells, ILT2-positive NK cells and ILT2-negative NK cells from human tumor cells.

Tumor target cells were HN (human oral squamous cell carcinoma, DMSZ® ACC 417, FaDu (human pharynx tissue, HNSCC, ATCC® HTB-43) or Cal27 (human tongue tissue, HNSCC, ATCC® CRL-2095™). Effector cells (fresh NK cells purified from human healthy donors) and tumor target cells were mixed at a ratio 1:1. The CD137 assay was carried out in 96 U well plates in completed RPMI, 200 μL final/well. Antibodies used included anti-ILT-2 antibodies 12D12, 18E1 and 26D8 at a concentration of 10 μg/mL, isotype control antibodies, in combination with cetuximab at a concentration of 0.01 μg/mL. Antibodies were pre-incubated 30 minutes at 37° C. with effector cells and then target cells were co-incubated overnight at 37° C. The following steps were: spin 3 min at 400 g; wash twice with Staining Buffer (SB); addition of 50 μL of staining Ab mix (anti-CD3 Pacific blue-BD Biosciences; anti-CD56-PE-Vio770-Miltenyi Biotec; anti-CD137-APC-Miltenyi Biotec; anti-ILT2-PE—clone HP—F1, eBioscience); incubation 30 min at 4° C.; wash twice with SB; resuspended pellet with Cellfix-Becton Dickinson; and fluorescence revealed with a FACS Canto II flow cytometer (Becton Dickinson). Negative controls were NK cells vs target cells alone and in presence of isotype control.

HNSCC tumor cells were found to be consistently negative for HLA-G and HLA-A2, as determined by flow cytometry, as shown in FIG. 12. However, despite the absence of the main known ligands of ILT2, the anti-ILT2 antibodies were able to mediate a strong increase of NK cell cytotoxicity mediated by cetuximab. The anti-ILT2 antibodies were able to mediate a strong increase of NK cell cytotoxicity mediated by cetuximab. Surprisingly, the combination of anti-ILT2 antibodies and cetuximab resulted in much stronger activation of total NK cell activation that either agent was able to mediate on its own. FIG. 11B shows the fold increase over cetuximab alone (compared to medium) in activation of NK cells following incubation with cetuximab with or without anti-ILT2 antibodies, and HN tumor target cells, in three human donors. FIG. 11C shows the fold increase over cetuximab alone (compared to medium) in activation of NK cells following incubation with cetuximab with or without anti-ILT2 antibodies, and FaDu tumor target cells, in three human donors. FIG. 11D shows the fold increase over cetuximab alone (compared to medium) in activation of NK cells following incubation with cetuximab with or without anti-ILT2 antibodies, and Cal27 tumor target cells, in three human donors. Each of the anti-ILT2 antibodies 12D12, 18E1 and 26D8 resulted in an increase of the NK cytotoxicity mediated by cetuximab alone. The combination increased NK cell cytotoxicity of cetuximab in the LILRB1+ population of NK cells and in the entire NK cell population.

### Example 14: Enhancement of Macrophage-Mediated ADCP

Antibodies were tested for the ability to enhance antibody-dependent cellular phagocytosis.

Briefly, monocyte derived macrophages from healthy donors were obtained after 6 to 7 days of culture in complete RPMI supplemented with 100 ng/mL of M-CSF in flat bottom 96 well plate (40000 cells/well). Antibody-dependent cell phagocytosis (ADCP) experiments were performed in RPMI without phenol red to avoid interference with the dye used to label target cells. Macrophages were starved in RPMI without FBS for 2 hours before addition of antibodies and target cells. A dose range of rituximab (10-1-0.1 μg/mL) and a fixed-dose of anti-ILT2 or control antibodies (10 μg/mL) were added on macrophages. 30000 cells/well HLA-A2-expressing target cells were labelled using ph-Rodo Red reagent (which is fluorescence at acidic pH in endocytic vesicles upon phagocytosis by macrophages), added to macrophages and incubated for 3 to 6 hours in the Incucyte-S3 imager. Images were acquired every 30 min. ADCP was quantified using total red objet integrated intensity (RCU× μm²/image) metrics.

Commercial anti-ILT2 antibody GHI/75 (mouse IgG2b isotype) and a variant (“HUB3”) thereof having human IgG1 Fc domains modified by introduction of the L234A/L235E/G237A/A330S/P331S mutations to substantially eliminate human FcγR binding were then tested for ability to increase rituximab-mediated phagocytosis by macrophages of HLA-A2-expressing B cells, compared to rituximab alone.

Results are shown in FIG. 13. The ILT2-blocking antibodies GHI/75 (commercial antibody, mouse IgG2b isotype) enhanced ADCP mediated by the anti-CD20 antibody rituximab in macrophages towards HLA-A2-expressing B cells (B104 cells). In comparison, the human IgG1 Fc-modified GHI/75 variant (HUB3 in FIG. 12) comprising the L234A/L235E/G237A/A330S/P331S mutations showed a decreased ability to enhance ADCP mediated by rituximab

The interactions between the Fc domain of anti-ILT2 antibodies and FcγR may therefore play an important role in the observed macrophage mediated cell death. This opens the possibility to modulate the ability of the anti-ILT2 antibodies to mediate ADCP through maintenance or inclusion of Fc domains that bind FcγR (e.g. native IgG1 domains) in order to mediate ADCP.

### Example 15: ILT2 in Urothelial Cancer

Potentiation of Cytotoxicity in Primary NK Cells from Urothelial Cancers Patients Towards HLA-A2-Expressing Cells

The effect of the anti-ILT2 antibodies on NK cell activation was determined by analysis by flow cytometry of CD137 expression on total NK cells, ILT2-positive NK cells and ILT2-negative NK cells from human urothelial carcinoma patients.

Effector cells were primary NK cells (fresh NK cells purified from human urothelial cancer donors, incubation overnight at 37° C. before use) and target cells (HLA-A2-expressing B cell line reference B104) were mixed at a ratio 1:1. The CD137 assay was carried out in 96 U well plates in completed RPMI, 200 μL final/well. Antibodies were pre-incubated 30 minutes at 37° C. with effector cells and then target cells were co-incubated overnight at 37° C. The following steps were: spin 3 min at 500 g; wash twice with Staining Buffer (SB); addition of 50 μL of staining Ab mix (anti-CD3 Pacific blue-BD Biosciences; anti-CD56-PE-Vio770-Miltenyi Biotec; anti-CD137-APC-Miltenyi Biotec; anti-ILT2-PE-clone HP—F1, eBioscience); incubation 30 min at 4° C.; wash twice with SB; resuspended pellet with SB; and fluorescence revealed with Canto II (HTS). Negative controls were NK cells vs target cells alone and in presence of isotype control.

FIG. 14 shows the % of ILT2-positive (right hand panel) and ILT2-negative (middle panel) NK cells from urothelial cancer patients expressing CD137 following incubation with anti-ILT2 antibodies 12D12, 18E1 and 26D8 and the HLA-A2-expressing B cells. Each of the anti-ILT2 antibodies 12D12, 18E1 and 26D8 caused a more than 2-fold increase in NK cell cytotoxicity.

### Example 16: ILT2 in Clear Cell Renal Carcinoma

Correlation of ILT2 Expression with Survival in Human CCRCC Patients

A study of ILT2 gene expression study was carried out using Cancer Genome Atlas (a collaboration between the National Cancer Institute and National Human Genome Research Institute) based on multi-dimensional maps of the key genomic changes in different types of cancer. Levels of expression (indicated as high or low) were considered, taking account of disease stage and time. For ILT2 and kidney clear cell renal cell carcinoma (CCRCC) patients were divided in 3 groups (high, mid and low ILT2 gene expression) according to the p-value of the Cox regression (each group must contain at least 10% of patients). Survival probability curves were drawn for each of the 3 groups. Statistical survival differences between low, mid and high ILT2 expression were observed for CCRCC samples, with high-expressing ILT2 exhibiting lower survival. FIG. 15 shows low ILT2 expressing samples (top line), medium ILT2-expressing samples (middle line) and high ILT2-expressing samples (bottom line). The results show that increased ILT2 expression correlates with lower survival probability. The high ILT2-expressing samples were associated with lower survival probability compared to medium and low ILT2 expressing samples.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

TABLE 7

| | | | | L234A/ | | | Human |
|---|---|---|---|---|---|---|---|
| | | | | L235E/ | L234A/ | Wild type | IgG4 |
| | | L234F/ | L234A/ | G237A/ | L235E/ | human | antibody |
| | | L235E/ | L235E/ | A330S/ | G237A/ | IgG1 | with |
| Human Fc | N297S | P331S | P331S | P331S | P331S | antibody | S241P |
| receptor | KD (nM) | KD (nM) | KD (nM) | KD (nM) | KD (nM) | KD (nM) | KD (nM) |
| CD64 | 278 | 933 | 1553 | No binding | No binding | 12.74 | 96.83 |
| CD32a | No binding | 14250 | 19900 | 18190 | 16790 | 2075 | 3218 |
| CD32b | No binding | 17410 | 79830 | 21800 | 16570 | 3914 | 2659 |
| CD16a(F) | No binding | 35580 | No binding | No binding | No binding | 961.9 | Low binding |
| CD16a(V) | No binding | 8627 | 9924 | No binding | No binding | 733.7 | 8511 |
| CD16b | No binding | No binding | No binding | No binding | No binding | 15020 | Low binding |
| FcRn | 712 | 627 | 1511 | 714 | 758 | 1272 | 1176 |

1-57. (canceled)

58. A monoclonal antibody or antibody fragment that specifically binds to a human ILT2 polypeptide and is capable of enhancing the cytotoxicity of NK cells in a cytotoxicity assay in which NK cells that express ILT2 are purified from human donors and incubated with target cells that express at their surface HLA-G polypeptides, wherein the antibody comprises:
a HCDR1 region (Kabat positions 31-35) of 2H2B comprising an amino acid sequence NYYMQ (SEQ ID NO: 139), optionally wherein one, two or three of these amino acids may be substituted by a different amino acid, optionally wherein the HCDR1 comprises an amino acid substitution at Kabat position 32, 33, 34 and/or 35, optionally wherein the HCDR1 comprises at least two aromatic residues (Y, H or F) at Kabat position 32, 33, 34 and/or 35, optionally wherein the HCDR1 comprises an aromatic residue (Y, H or F) at Kabat position 32 and/or an aromatic residue (Y, H or F), N or Q at 35;
a HCDR2 region (Kabat positions 50-65) of 2H2B comprising an amino acid sequence WIFPGSGESSYNEKFKG (SEQ ID NO: 140) or WIFPGSGESNYNEKFKG (SEQ ID NO: 161), optionally wherein one, two or three of these amino acids may be substituted by a different amino acid, optionally wherein the HCDR2 comprises an amino acid substitution at Kabat position 52A, 54, 55, 56, 57, 58, 60 and/or 65, optionally wherein the residue at 52A is P or L, optionally wherein the residue at 54 is G, S, N or T, optionally wherein the residue at 55 is G, N or Y, optionally wherein the residue at 56 is E or D, optionally wherein the residue at 57 is S or T, optionally wherein the residue at 58 is S, K or N, optionally wherein the residue at 60 is N or S, optionally wherein the residue at 65 is G or V;
a HCDR3 region (Kabat positions 95-102) of 2H2B comprising an amino acid sequence TWNYDARWGY (SEQ ID NO: 141), optionally wherein one, two or three of these amino acids may be substituted by a different amino acid, optionally wherein the HCDR3 comprises an amino acid substitution at Kabat position 95, optionally wherein the residue at 95 is T or S, optionally wherein the HCDR3 comprises an amino acid substitution at Kabat position 101, optionally wherein the residue at 101 is G or V;
a Kabat LCDR1 region (Kabat positions 34-34) of 2H2B comprising an amino acid sequence IPSESIDSYGISFMH (SEQ ID NO: 142), optionally wherein one, two or three of these amino acids may be substituted by a different amino acid, optionally wherein the LCDR1 comprises an amino acid substitution at Kabat position 24, 25, 26, 27, 27A, 28, 33 and/or 34, optionally wherein the residue at 24 is I or R, optionally wherein the residue at 25 is A, P or V, optionally wherein the residue at 26 is S or N, optionally wherein the residue at 27 is E or D, optionally wherein the residue at 27A is S, G, T, I or N, optionally wherein the residue at 28 is Y or F, optionally wherein the residue at 33 is M, I or L, optionally wherein the residue at 34 is H or S, optionally wherein the LCDR1 comprises an amino acid deletion at Kabat position 29, 30 31 and/or 32;
a Kabat LCDR2 region (Kabat positions 50-56) of 2H2B comprising an amino acid sequence RASNLES (SEQ ID NO: 143), optionally wherein, two or three of these amino acids may be substituted by a different amino acid, optionally wherein one or more of these amino acids may be substituted by a different amino acid, optionally wherein the LCDR2 comprises an amino acid substitution at Kabat position 50, 53 and/or 55, optionally wherein the residue at 50 is R or G, optionally wherein the residue at 53 is N, T or I, optionally wherein the residue at 55 is D, E or V;
a Kabat LCDR3 region (Kabat positions 89-97) of 2H2B comprising an amino acid sequence QQSNEDPFT (SEQ ID NO: 144), optionally wherein one, two or three of these amino acids may be deleted or substituted by a different amino acid, optionally wherein the LCDR3 comprises an amino acid substitution at Kabat position 91, 94 and/or 96, optionally wherein the residue at 91 is S or T, optionally wherein the residue at 94 is D or A, optionally wherein the residue at 96 is F or W.

59. The antibody of claim 58, wherein the antibody comprises a HCDR1 comprising an amino acid sequence NYYMQ (SEQ ID NO: 139); a HCDR2 comprising an amino acid sequence WIFPGSGESSYNEKFKG (SEQ ID NO: 140); a HCDR3 comprising an amino acid sequence TWNYDARWGY (SEQ ID NO: 141); a LCDR1 comprising an amino acid sequence IPSESIDSYGISFMH (SEQ ID NO: 142); a LCDR2 region comprising an amino acid sequence RASNLES (SEQ ID NO: 143); and a LCDR3 region comprising an amino acid sequence QQSNEDPFT (SEQ ID NO: 144), wherein any of said light or heavy chain CDRs may contain one, two or three amino acid modifications.

60. The antibody of claim 58, wherein the antibody comprises a HCDR1 comprising an amino acid sequence NYYVQ (SEQ ID NO: 151); a HCDR2 comprising an amino acid sequence WIFPGSGETNYNEKFKA (SEQ ID NO: 152); a HCDR3 comprising an amino acid sequence TWNYDARWGY (SEQ ID NO: 141); a LCDR1 comprising an amino acid sequence RPSENIDSYGISFMH (SEQ ID NO: 181); a LCDR2 region comprising an amino acid sequence RASNLES (SEQ ID NO: 149); and a LCDR3 region comprising an amino acid sequence QQTNEDPFT (SEQ ID NO: 153).

61. The antibody of claim 58, wherein the antibody comprises a HCDR1 comprising an amino acid sequence NYYIH (SEQ ID NO: 163); a HCDR2 comprising an amino acid sequence WIFPGSGETNYNEKFKV (SEQ ID NO: 164); a HCDR3 comprising an amino acid sequence TWNYDARWGY (SEQ ID NO: 141); a LCDR1 comprising an amino acid sequence RASESIDSYGISFMH (SEQ ID NO: 165); a LCDR2 region comprising an amino acid sequence RASNLES (SEQ ID NO: 149); and a LCDR3 region comprising an amino acid sequence QQSNEDPFT (SEQ ID NO: 150).

62. An antibody that is capable of binding a human ILT2 protein, wherein the antibody is selected from the group consisting of:
(a) an antibody comprising (i) a heavy chain CDR 1, 2 and 3 of the heavy chain variable region of SEQ ID NO: 12 and (ii) a light chain CDR 1, 2 and 3 of the light chain variable region of SEQ ID NO: 13;
(b) an antibody comprising (i) a heavy chain CDR 1, 2 and 3 of the heavy chain variable region of SEQ ID NO: 20 and (ii) a light chain CDR 1, 2 and 3 of the light chain variable region of SEQ ID NO: 21; and
(c) an antibody comprising (i) a heavy chain CDR 1, 2 and 3 of the heavy chain variable region of SEQ ID NO: 28 and (ii) a light chain CDR 1, 2 and 3 of the light chain variable region of SEQ ID NO: 29.

63. An antibody that is capable of binding a human ILT2 protein, wherein the antibody is selected from the group consisting of:
(a) an antibody comprising (i) a heavy chain CDR 1, 2 and 3 of the heavy chain variable region of SEQ ID NO: 93 and (ii) a light chain CDR 1, 2 and 3 of the light chain variable region of SEQ ID NO: 94;
(b) an antibody comprising (i) a heavy chain CDR 1, 2 and 3 of the heavy chain variable region of SEQ ID NO: 95 and (ii) a light chain CDR 1, 2 and 3 of the light chain variable region of SEQ ID NO: 96; and
(c) an antibody comprising (i) a heavy chain CDR 1, 2 and 3 of the heavy chain variable region of SEQ ID NO: 83 and (ii) a light chain CDR 1, 2 and 3 of the light chain variable region of SEQ ID NO: 84.

64. The antibody of claim 63, wherein the VH comprises: an amino acid substitution at Kabat position 32, 33, 34 and/or 35; an amino acid substitution at Kabat position 52A, 54, 55, 56, 57, 58, 60 and/or 65; and/or an amino acid substitution at Kabat position 95 and/or 101.

65. The antibody of claim 64, wherein the VL comprises: an amino acid substitution at Kabat position 24, 25, 26, 27, 27A, 28, 33 and/or 34, and/or an amino acid deletion at Kabat position 29, 30, 31 and/or 32; an amino acid substitution at Kabat position 50, 53 and/or 55; and/or an amino acid substitution at Kabat position 91, 94 and/or 96.

66. The antibody of claim 63, wherein the antibody comprises a HCDR1 comprising an amino acid sequence NYYMQ (SEQ ID NO: 139); a HCDR2 comprising an amino acid sequence WIFPGSGESSYNEKFKG (SEQ ID NO: 140); a HCDR3 comprising an amino acid sequence TWNYDARWGY (SEQ ID NO: 141); a LCDR1 comprising an amino acid sequence IPSESIDSYGISFMH (SEQ ID NO: 142); a LCDR2 region comprising an amino acid sequence RASNLES (SEQ ID NO: 143); and a LCDR3 region comprising an amino acid sequence QQSNEDPFT (SEQ ID NO: 144), wherein any of said light or heavy chain CDRs may contain one, two or three amino acid modifications.

67. The antibody of claim 63, wherein the antibody comprises a HCDR1 comprising an amino acid sequence NYYVQ (SEQ ID NO: 151); a HCDR2 comprising an amino acid sequence WIFPGSGETNYNEKFKA (SEQ ID NO: 152); a HCDR3 comprising an amino acid sequence TWNYDARWGY (SEQ ID NO: 141); a LCDR1 comprising an amino acid sequence RPSENIDSYGISFMH (SEQ ID NO: 181); a LCDR2 region comprising an amino acid sequence RASNLES (SEQ ID NO: 149); and a LCDR3 region comprising an amino acid sequence QQTNEDPFT (SEQ ID NO: 153).

68. The antibody of claim 63, wherein the antibody comprises a HCDR1 comprising an amino acid sequence NYYIH (SEQ ID NO: 163); a HCDR2 comprising an amino acid sequence WIFPGSGETNYNEKFKV (SEQ ID NO: 164); a HCDR3 comprising an amino acid sequence TWNYDARWGY (SEQ ID NO: 141); a LCDR1 comprising an amino acid sequence RASESIDSYGISFMH (SEQ ID NO: 165); a LCDR2 region comprising an amino acid sequence RASNLES (SEQ ID NO: 149); and a LCDR3 region comprising an amino acid sequence QQSNEDPFT (SEQ ID NO: 150).

69. The antibody of claim 63, wherein the antibody has reduced binding to a mutant ILT2 polypeptide comprising the mutations E34A, R36A, Y76I, A82S, R84L (with reference to SEQ ID NO: 2), in each case relative to binding between the antibody and a wild-type ILT2 polypeptide comprising the amino acid sequence of SEQ ID NO: 2.

70. The antibody of claim 63, wherein the antibody comprises a modified human IgG1 Fc domain comprising N-linked glycosylation at Kabat residue N297 and comprising an amino acid substitution at Kabat residue(s) 234 and 235, optionally further at Kabat residue 331, optionally at Kabat residues 234, 235, 237 and at Kabat residues 330 and/or 331, optionally wherein the Fc domain comprises L234A/L235E/P331S substitutions, L234F/L235E/P331S substitutions, L234A/L235E/G237A/P331S substitutions, or L234A/L235E/G237A/A330S/P331S substitutions.

71. A pharmaceutical composition comprising an antibody according to claim 63, and a pharmaceutically acceptable carrier.

72. A kit comprising the antibody of claim 63, optionally further comprising a labelled secondary antibody that specifically recognizes said antibody.

73. A nucleic acid or set of nucleic acids encoding a heavy and/or light chain of an antibody of claim 63.

74. A hybridoma or recombinant host cell producing the antibody of claim 63.

75. A method for the treatment of cancer in a patient having a cancer selected form a urothelial carcinoma, a head and neck squamous cell carcinoma (HNSCC), a lung cancer, an NSCLC, a renal cell carcinoma and an ovarian cancer, the method comprising administering to said patient an effective amount of an antibody that binds a human ILT2 polypeptide and which is capable of neutralizing the inhibitory activity of an ILT2 polypeptide in an NK and/or CD8 T cell.

76. In a method of treating a tumor in a human individual by administering an antibody that binds a tumor-associated antigen and mediates antibody-dependent cell-mediated cytotoxicity (ADCC), the improvement comprising further administering to the individual an effective amount of an antibody of claim 58.

77. A method of treating a tumor in a human individual, the treatment comprising administering to the individual an effective amount of each of: (a) a means for inducing the NK-cell mediated ADCC of tumor cells, and (b) a means for neutralizing the inhibitory activity a human ILT2 protein without binding to the human Fcγ receptor CD16A, wherein the means for neutralizing the inhibitory activity a human ILT2 protein without binding to the human Fcγ receptor CD16A is an antibody of claim 58.

78. In a method of treating a tumor in a human individual by administering an agent or treatment that neutralizes the inhibitory activity a human ILT2 domain protein, the improvement comprising administering to the individual an effective amount of means for binding an epitope within the segment of amino acid residues of the ILT2 polypeptide defined by the sequence shown in SEQ ID NO: 55, wherein the means for binding an epitope within the segment of amino acid residues of the ILT2 polypeptide defined by the sequence shown in SEQ ID NO: 55 is an antibody of claim 58.

79. A method for the treatment or prevention of a urothelial cancer, a head and neck squamous cell carcinoma (HNSCC), a NSCLC, a renal cell cancer or an ovarian cancer in a patient in need thereof, the method comprising administering to said patient an effective amount of an antibody of claim 58.

80. The method of claim 79, wherein the individual has a tumor characterized by ILT2-expressing NK and/or CD8 T cells, optionally wherein the cells have high levels of ILT2 expressed at their surface.

81. A method for stimulating an adaptive immune response, optionally a method for stimulating a CD8+ T cell response, in a subject having a cancer, the method comprising administering to said subject an effective amount of an antibody of claim 58.

82. A method for modulating the activity of monocyte-derived cells and/or lymphocytes, optionally NK cells and/or CD8+ T cells, in a subject having a cancer, the method comprising administering to said subject an effective amount of an antibody of claim 58.

83. A method for selecting a subject having a cancer that responds to a treatment with an antibody of claim 58, the method comprising determining whether cancer cells in said subject express HLA-A2 and/or HLA-G, the expression of HLA-A2 and/or HLA-G being indicative of a responder subject, and administering to a responder subject said antibody.
